(19) United States
(12) Patent Application Publication
(10) Pub. No.: US 2003/0216257 A1
(43) Pub. Date: Nov. 20, 2003

(54) 1-aryl-4-alkyl halide-2(1h)-pyridones and their use as herbicides

(76) Inventors: Ingo Sagasser, Dannstadt-Schauernheim (DE); Olaf Menke, Altleiningen (DE); Gerhard Hamprecht, Weinheim (DE); Michael Puhl, Lampertheim (DE); Robert Reinhard, Ludwigshafen (DE); Matthias Witschel, Bad Durkheim (DE); Cyrill Zagar, Ludwigshafen (DE); Helmut Walter, Obrigheim (DE)

Correspondence Address: KEIL & WEINKAUF, 1350 CONNECTICUT AVENUE, N.W., WASHINGTON, DC, 20036, US

(21) Appl. No.: 10/332,860

(22) PCT Filed: Jul. 17, 2001
(86) PCT No.: PCT/EP01/08251

(30) Foreign Application Priority Data
Jul. 18, 2000 (DE) 10034838.6

(51) Int. Cl. A01N 43/40; C07D 213/62; C07D 213/63; C 07D 4 1/04

(52) U.S. Cl. 504/250; 504/254; 546/261; 546/288; 546/290

(57) ABSTRACT

The use of 1-aryl-4-haloalkyl-2-[1H]-pyridones of the formula I

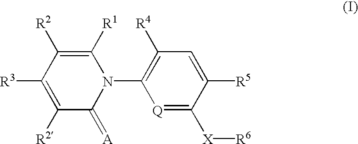

in which the variables are as defined in claim 1, and their use as herbicides, is described.

The present invention relates to the use of 1-aryl-4-haloalkyl-2-[1H]pyridones and of their agriculturally useful salts as herbicides, desiccants or defoliants.

In various publications, 1-aryl-2-[1H]pyridones have been described as active substances in compositions for controlling animal pests (pesticides). EP-A 272 824, for example, relates to pesticides comprising, as active compound, 1-(2-pyridyl)-2-[1H]pyridones. Described are, inter alia, 1-(2-pyridyl)-2-[1H]pyridones of the formula

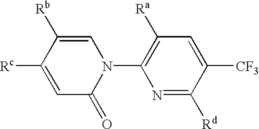

in which

R^{a} is hydrogen, chlorine, bromine, nitro, amino or trifluoromethyl;

R^{b} is hydrogen, chlorine, bromine or trifluoromethyl;

R^{c} is C₁-C₄-haloalkyl; and

R^{d} is preferably hydrogen.

EP-A 259 048 describes pesticides based on 1-phenyl-2-[1H]pyridones which, preferably, carry a halogen atom in the 2- and the 6-position of the phenyl ring.

WO 99/55668 describes insecticidally and miticidally acting compounds of the formula

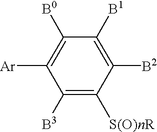

in which

R is alkyl, alkenyl, alkynyl or a comparable radical,

B⁰ to B³, independently of one another, are hydrogen, halogen, cyano haloalkyl or comparable radicals;

n is 0, 1 or 2; and

Ar is an aromatic radical, inter alia a 1H-2-pyridon-1-yl radical.

EP-A 488220 describes herbicidally acting compounds of the formula

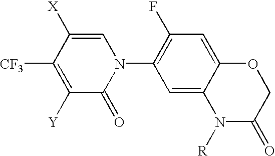

in which

R is, inter alia, alkyl, alkenyl, alkynyl, alkoxyalkyl, haloalkyl or a comparable radical,

X is hydrogen, halogen, methyl or ethyl which may be substituted by halogen; and

Y is hydrogen or methyl.

In principle, there is a constant need for novel herbicidally active substances to be provided, in order to circumvent a possible formation of resistance against known herbicides.

It is an object of the present invention to provide novel herbicides which allow better control of harmful plants than the herbicides of the prior art. Advantageously, the novel herbicides should have high activity against harmful plants. Moreover, crop plant compatibility is desirable.

This object is achieved by the 1-aryl-4-haloalkyl-2-[1H]pyridones of the formula I defined below.

Accordingly, the present invention relates to the use of 1-aryl-4-haloalkyl-2-[1H]pyridones of the formula I

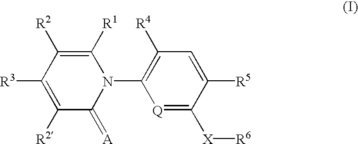

in which variables A, X, Q, R¹, R², R^{2′}, R³, R⁴, R⁵ and R⁶ are as defined below:

R¹ is hydrogen or halogen;

R² and R^{2′} independently of one another are hydrogen, amino or C₁-C₄-alkyl;

R³ is C₁-C₄-haloalkyl;

R⁴ is hydrogen or halogen;

R⁵ is hydrogen, cyano, nitro, halogen, C₁-C₄-alkyl, C₁-C₄-haloalkyl, C₁-C₄-alkoxy or C₁-C₄-haloalkoxy;

A is oxygen or sulfur;

X is a chemical bond, methylene, 1,2-ethylene, propane-1,3-diyl, ethene-1,2-diyl, ethyne-1,2-diyl or is oxymethylene or thiamethylene, attached to the phenyl ring via the heteroatom, where all groups may be unsubstituted or may carry one or two substituents, in each case selected from the group consisting of cyano, carboxyl, halogen, C₁-C₄-alkyl, C₁-C₄-haloalkyl, C₁-C₄-alkoxy, (C₁-C₄-alkoxy)carbonyl, di(C₁-C₄-alkyl)amino and phenyl;

R⁶ is hydrogen, nitro, cyano, halogen, halosulfonyl, —O—Y—R⁸, —O—CO—Y—R⁸, —N(Y—R⁸)(Z—R⁹), —N(Y—R⁸)—SO₂—Z—R⁹, —N(SO₂—Y—R⁸)(SO₂—Z—R⁹), —N(Y—R⁸)—CO—Z—R⁹, —N(Y—R⁸)(O—Z—R⁹), —S(O)ₙ—Y—R⁸ where n=0, 1 or 2, —SO₂—O—Y—R⁸, —SO₂—N(Y—R⁸)(Z—R⁹), —CO—Y—R⁸, —C(═NOR¹⁰)—Y—R⁸, —C(═NOR¹⁰)—O—Y—R⁸, —CO—O—Y—R⁸, —CO—S—Y—R⁸, —CO—N(Y—R⁸)(Z—R⁹), —CO—N(Y—R⁸)(O—Z—R⁹) or —PO(O—Y—R⁸)₂;

Q is nitrogen or a group C—R⁷ in which R⁷ is hydrogen, OH, SH or NH₂; or

X—R⁶ and R⁷ are a 3- or 4-membered chain whose chain members may, in addition to carbon, include 1, 2 or 3 heteroatoms selected from the group of nitrogen, oxygen and sulfur atoms, which may be unsubstituted or may for their part carry one, two or three substituents and whose members may also include one or two nonadjacent carbonyl, thiocarbonyl or sulfonyl groups,

where the variables Y, Z, R⁸, R⁹ and R¹⁰ are as defined below:

Y, Z independently of one another are:

a chemical bond, methylene or 1,2-ethylene, which may be unsubstituted or may carry one or two substituents, in each case selected from the group consisting of carboxyl,

C₁-C₄-alkyl, C₁-C₄-haloalkyl, (C₁-C₄-alkoxy)carbonyl and phenyl;

R⁸, R⁹ independently of one another are:

hydrogen, C₁-C₆-haloalkyl, C₁-C₄-alkoxy-C₁-C₄-alkyl, C₂-C₆-alkenyl, C₂-C₆-haloalkenyl, C₂-C₆-alkynyl, C₂-C₆-haloalkynyl, —CH(R¹¹)(R¹²), —C(R¹¹)(R¹²)—NO₂, —C(R¹¹)(R¹²)—CN, —C(R¹¹)(R¹²)-halogen, —C(R¹¹)(R¹²)—OR¹³, —C(R¹¹)(R¹²)—N(R¹³)R¹⁴, —C(R¹¹)(R¹²)—N(R¹³)—OR¹⁴, —C(R¹¹)(R¹²)—SR¹³, —C(R¹¹)(R¹²)—SO—R¹³, —C(R¹¹)(R¹²)—SO₂—R¹³, —C(R¹¹)(R¹²)—SO₂—OR¹³, —C(R¹¹)(R¹²)—SO₂—N(R¹³)R¹⁴, —C(R¹¹)(R¹²)—CO—R¹³, —C(R¹¹)(R¹²)—C(═NOR¹⁵)—R¹³, —C(R¹¹)(R¹²)—CO—OR¹³, —C(R¹¹)(R¹²)—CO—SR¹³, —C(R¹¹)(R¹²)—C—N(R¹³)R¹⁴, —C(R¹¹)(R¹²)—CO—N(R¹³)—OR¹⁴, —C(R¹¹)(R¹²)—PO(OR¹³)₂, C₃-C₈-cycloalkyl-C₁-C₄-alkyl, C₃-C₈-cycloalkyl which may contain a carbonyl or thiocarbonyl ring member,

phenyl or 3-, 4-, 5-, 6- or 7-membered heterocyclyl which may contain a carbonyl or thiocarbonyl ring member, where each cycloalkyl, the phenyl and each heterocyclyl ring may be unsubstituted or may carry one, two, three or four substituents, in each case selected from the group consisting of cyano, nitro, amino, hydroxyl, carboxyl, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfonyl, C₁-C₄-haloalkylsulfonyl, (C₁-C₄-alkyl)carbonyl, (C₁-C₄-haloalkyl)carbonyl, (C₁-C₄-alkyl)carbonyloxy, (C₁-C₄-haloalkyl)carbonyloxy, (C₁-C₄-alkoxy)carbonyl and di(C₁-C₄-alkyl)amino;

R¹⁰ is hydrogen, C₁-C₆-alkyl, C₁-C₆-haloalkyl, C₁-C₄-alkoxycarbonyl-C₁-C₄-alkyl, C₂-C₆-alkenyl, C₂-C₆-haloalkenyl, C₂-C₆-alkynyl, C₂-C₆-haloalkynyl, C₃-C₈-cycloalkyl, phenyl or phenyl-C₁-C₄-alkyl;

where the variables R¹¹ to R¹⁵ are as defined below:

R¹¹, R¹² independently of one another are hydrogen, C₁-C₄-alkyl, C₁-C₄-alkoxy-C₁-C₄-alkyl, C₁-C₄-alkylthio-C₁-C₄-alkyl, (C₁-C₄-alkoxy)carbonyl-C₁-C₄-alkyl or phenyl-C₁-C₄-alkyl, where the phenyl ring may be unsubstituted or may carry one to three substituents, in each case selected from the group consisting of cyano, nitro, carboxyl, halogen, C₁-C₄-alkyl, C₁-C₄-haloalkyl and (C₁-C₄-alkoxy)carbonyl;

R¹³, R¹⁴ independently of one another are

hydrogen, C₁-C₆-alkyl, C₁-C₆-haloalkyl, C₁-C₄-alkoxy-C₁-C₄-alkyl, C₂-C₆-alkenyl, C₂-C₆-haloalkenyl, C₂-C₆-alkynyl, C₂-C₆-haloalkynyl, C₃-C₈-cycloalkyl, C₃-C₈-cycloalkyl-C₁-C₄-alkyl, phenyl, phenyl-C₁-C₄-alkyl, 3- to 7-membered heterocyclyl or heterocyclyl-C₁-C₄-alkyl, where each cycloalkyl and each heterocyclyl ring may contain a carbonyl or thiocarbonyl ring member,

and where each cycloalkyl, the phenyl and each heterocyclyl ring may be unsubstituted or may carry one to four substituents, in each case selected from the group consisting of cyano, nitro, amino, hydroxyl, carboxyl, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfonyl, C₁-C₄-haloalkylsulfonyl, (C₁-C₄-alkyl)carbonyl, (C₁-C₄-haloalkyl)carbonyl, (C₁-C₄-alkyl)carbonyloxy, (C₁-C₄-haloalkyl)carbonyloxy, (C₁-C₄-alkoxy)carbonyl and di(C₁-C₄-alkyl)amino;

R¹⁵ is hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, phenyl or phenyl-C₁-C₄-alkyl;

and their agriculturally useful salts as herbicides or for the desiccation/defoliation of plants.

The present invention furthermore relates to the compounds of the formula I defined above and their agriculturally useful salts, compounds of the formula I in which A is oxygen, Q is CH, R³ and R⁵ are trifluoromethyl and R¹, R², R^{2′}, R⁴ and X—R⁶ are hydrogen; or in which A is oxygen and Q is N, R³ and R⁴ are as defined above, R¹, R² and R^{2′} are hydrogen and X—R⁶ is hydrogen or halogen, if R⁵ is trifluoromethyl, being excluded from the compounds that are claimed. Also excluded are compounds of the formula I in which A is oxygen, Q is CH and R³ is trifluoromethyl, R¹, R², R^{2′}, R⁴ are hydrogen and X—R⁶ is a group S(O)ₙ—Y—R⁸ where n=0, 1 or 2, in which Y is a single bond and R⁸ is selected from the group consisting of n-propyl, isopropyl, cyclopropylmethyl and 2,2,2-trifluoroethyl.

The invention furthermore relates to:

herbicidal compositions and compositions for the desiccation and/or defoliation of plants, the compositions comprising, as active substances, the compounds I,

processes for preparing the compounds I and herbicidal compositions and compositions for the desiccation and/or defoliation of plants using the compounds I, and also

methods for controlling undesirable vegetation (harmful plants) and for the desiccation and/or defoliation of plants using the compounds I,

compounds of the formula II

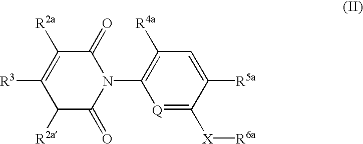

in which R³, X and Q are as defined above and R^{2a}, R^{2a′}, R^{4a}, R^{5a}, R^{6a} are R², R^{2′}, R⁴ R⁵ and R⁶ as defined above, except for compounds of the formula II, in which Q is CH, R³ and R^{5a} are trifluoromethyl and R^{2a}, R^{2a′}, R^{4a} and X—R^{6a} are hydrogen;

furthermore except for compounds of the formula II in which Q is N, R³ and R^{4a} have the meanings given above for R³ and R⁴, respectively, R^{2a} and R^{2a′} are hydrogen, X—R^{6a} is hydrogen or halogen, if R^{5a} is trifluoromethyl,

furthermore except for compounds of the formula II where Q=CH and R³ =trifluoromethyl, if R^{2a}, R^{2a′} and R^{4a} are hydrogen, R^{5a} has the meaning given for R⁵ in claim 1, X is a single bond and R⁶ is a group S(O)ₙ—YR⁸ where n=0, 1 or 2, where Y is a single bond and R⁸ is selected from the group consisting of n-propyl, isopropyl, cyclopropylmethyl and 2,2,2-trifluoroethyl, and the tautomers of the compounds II.

In the substituents, the compounds of the formula I may have one or more centers of chirality, in which case they are present as mixtures of enantiomers or diastereomers. The present invention provides both the pure enantiomers or diastereomers and mixtures thereof.

Agriculturally useful salts are especially the salts of those cations and the acid addition salts of those acids whose cations and anions, respectively, do not have any adverse effect on the herbicidal activity of the compounds I. Thus, suitable cations are, in particular, the ions of the alkali metals, preferably sodium and potassium, the alkali earth metals, preferably calcium, magnesium and barium, and the transition metals, preferably manganese, copper, zinc and iron, and also the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting I with an acid of the corresponding anion, preferably hydrochloric acid, hydrobromic acid, sulfuric acid, phosphoric acid or nitric acid.

The organic moieties mentioned in the definitions of substituents R², R^{2′}, R⁴, R⁵, R⁶, R⁷ to R¹⁹ or as radicals on cycloalkyl, phenyl or heterocyclic rings are—like the term halogen—collective terms for individual listings of the individual group members. All carbon chains, i.e. all alkyl, haloalkyl, alkoxy, haloalkoxy, alkylthio, haloalkylthio, alkylsulfinyl, haloalkylsulfinyl, alkylsulfonyl, haloalkylsulfonyl, alkenyl, haloalkenyl, alkynyl and haloalkynyl groups, and the corresponding moieties in larger groups, such as alkoxycarbonyl, phenylalkyl, cycloalkylalkyl, alkoxycarbonylalkyl, etc., can be straight-chain or branched, where the prefix Cₙ-Cₘ indicates in each case the possible number of carbon atoms in the group. Halogenated substituents preferably carry one, two, three, four or five identical or different halogen atoms. The term halogen represents in each case fluorine, chlorine, bromine or iodine.

Other examples of meanings are:

C₁-C₄-alkyl: CH₃, C₂H₅, n-propyl, CH(CH₃)₂, n-butyl, CH(CH₃)—C₂H₅, CH₂—CH(CH₃)₂ and C(CH₃)₃;

C₁-C₄-haloalkyl: a C₁-C₄-alkyl radical as mentioned above which is partially or fully substituted by fluorine, chlorine, bromine and/or iodine, i.e. for example CH₂F, CHF₂, CF₃, CH₂Cl, dichloromethyl, trichloromethyl, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, C₂F₅, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-fluoromethyl-2-fluoroethyl, 1-chloromethyl-2-chloroethyl, 1-bromomethyl-2-bromoethyl, 4-fluorobutyl, 4-chlorobutyl, 4-bromobutyl and nonafluorobutyl;

C₁-C₆-alkyl: C₁-C₄-alkyl as mentioned above, and also, for example, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl or 1-ethyl-2-methylpropyl, preferably methyl, ethyl, n-propyl, 1-methylethyl, n-butyl, 1,1-dimethylethyl, n-pentyl or n-hexyl;

C₁-C₆-haloalkyl: a C₁-C₆-alkyl radical as mentioned above which is partially or fully substituted by fluorine, chlorine, bromine and/or iodine, i.e. for example one of the radicals mentioned under C₁-C₄-haloalkyl, and also 5-fluoro-1-pentyl, 5-chloro-1-pentyl, 5-bromo-1-pentyl, 5-iodo-1-pentyl, 5,5,5-trichloro-1-pentyl, undecafluoropentyl, 6-fluoro-1-hexyl, 6-chloro-1-hexyl, 6-bromo-1-hexyl, 6-iodo-1-hexyl, 6,6,6-trichloro-1-hexyl or dodecafluorohexyl;

phenyl-C₁-C₄-alkyl: benzyl, 1-phenylethyl, 2-phenylethyl, 1-phenylprop-1-yl, 2-phenylprop-1-yl, 3-phenylprop-1-yl, 1-phenylbut-1-yl, 2-phenylbut-1-yl, 3-phenylbut-1-yl, 4-phenylbut-1-yl, 1-phenylbut-2-yl, 2-phenylbut-2-yl, 3-phenylbut-2-yl, 4-phenylbut-2-yl, 1-phenylmethyleth-1-yl, 1-phenylmethyl-1-methyleth-1-yl or 1-phenylmethylprop-1-yl, preferably benzyl or 2-phenylethyl;

heterocyclyl-C₁-C₄-alkyl: heterocyclylmethyl, 1-heterocyclylethyl, 2-heterocyclylethyl, 1-heterocyclylprop-1-yl, 2-heterocyclylprop-1-yl, 3-heterocyclylprop-1-yl, 1-heterocyclylbut-1-yl, 2-heterocyclylbut-1-yl, 3-heterocyclylbut-1-yl, 4-heterocyclylbut-1-yl, 1-heterocyclylbut-2-yl, 2-heterocyclylbut-2-yl, 3-heterocyclylbut-2-yl, 3-heterocyclylbut-2-yl, 4-heterocyclylbut-2-yl, 1-heterocyclylmethyleth-1-yl, 1-heterocyclylmethyl-1-methyleth-1-yl or 1-heterocyclylmethylprop-1-yl, preferably heterocyclylmethyl or 2-heterocyclylethyl;

C₁-C₄-alkoxy: OCH₃, OC₂H₅, n-propoxy, OCH(CH₃)₂, n-butoxy, OCH(CH₃)—C₂H₅, OCH₂—CH(CH₃)₂ or OC(CH₃)₃, preferably OCH₃, OC₂H₅, or OCH(CH₃)₂;

C₁-C₄-haloalkoxy: a C₁-C₄-alkoxy radical as mentioned above which is partially or fully substituted by fluorine, chlorine, bromine and/or iodine, i.e. for example OCH₂F, OCHF₂, OCF₃, OCH₂Cl, OCH(Cl)₂, OC(Cl)₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, 2,2,3,3,3-pentafluoropropoxy, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy, preferably OCHF₂, OCF₃, dichlorofluoromethoxy, chlorodifluoromethoxy or 2,2,2-trifluoroethoxy;

C₁-C₆-alkylthio: SCH₃, SC₂H₅, n-propylthio, SCH(CH₃)₂, n-butylthio, SCH(CH₃)—C₂H₅, SCH₂—CH(CH₃)₂ or SC(CH₃)₃, preferably SCH₃ or SC₂H₅;

C₁-C₄-haloalkylthio: a C₁-C₄-alkylthio radical as mentioned above which is partially or fully substituted by fluorine, chlorine, bromine and/or iodine, i.e. for example SCH₂F, SCHF₂, SCH₂Cl, SCH(Cl)₂, SC(Cl)₃, SCF₃, chlorofluoromethylthio, dichlorofluoromethylthio, chlorodifluoromethylthio, 2-fluoroethylthio, 2-chloroethylthio, 2-bromoethylthio, 2-iodoethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio, SC₂F₅, 2-fluoropropylthio, 3-fluoropropylthio, 2,2-difluoropropylthio, 2,3-difluoropropylthio, 2-chloropropylthio, 3-chloropropylthio, 2,3-dichloropropylthio, 2-bromopropylthio, 3-bromopropylthio, 3,3,3-trifluoropropylthio, 3,3,3-trichloropropylthio, SCH₂—C₂F₅, SCF₂-—C₂F₅, 1-(CH₂F)-2-fluoroethylthio, 1-(CH₂Cl)-2-chloroethylthio, 1-(CH₂Br)-2-bromoethylthio, 4-fluorobutylthio, 4-chlorobutylthio, 4-bromobutylthio or SCF₂—CF₂—C₂F₅, preferably SCHF₂, SCF₃, dichlorofluoromethylthio, chlorodifluoromethylthio or 2,2,2-trifluoroethylthio;

C₁-C₄-alkoxy-C₁-C₄-alkyl: C₁-C₄-alkyl which is substituted by C₁-C₄-alkoxy as mentioned above, i.e. for example CH₂—OCH₃, CH₂—OC₂H₅, n-propoxymethyl, CH₂—OCH(CH₃)₂, n-butoxymethyl, (1-methylpropoxy)methyl, (2-methylpropoxy)methyl, CH₂—OC(CH₃)₃, 2-(methoxy)ethyl, 2-(ethoxy)ethyl, 2-(n-propoxy)ethyl, 2-(1-methylethoxy)ethyl, 2-(n-butoxy)ethyl, 2-(1-methylpropoxy)ethyl, 2-(2-methylpropoxy)ethyl, 2-(1,1-dimethylethoxy)ethyl, 2-(methoxy)propyl, 2-(ethoxy)propyl, 2-(n-propoxy)propyl, 2-(1-methylethoxy)propyl, 2-(n-butoxy)propyl, 2-(1-methylpropoxy)propyl, 2-(2-methylpropoxy)propyl, 2-(1,1-dimethylethoxy)propyl, 3-(methoxy)propyl, 3-(ethoxy)propyl, 3-(n-propoxy)propyl, 3-(1-methylethoxy)propyl, 3-(n-butoxy)propyl, 3-(1-methylpropoxy)propyl, 3-(2-methylpropoxy)propyl, 3-(1,1-dimethylethoxy)propyl, 2-(methoxy)butyl, 2-(ethoxy)butyl, 2-(n-propoxy)butyl, 2-(1-methylethoxy)butyl, 2-(n-butoxy)butyl, 2-(1-methylpropoxy)butyl, 2-(2-methylpropoxy)butyl, 2-(1,1-dimethylethoxy)butyl, 3-(methoxy)butyl, 3-(ethoxy)butyl, 3-(n-propoxy)butyl, 3-(1-methylethoxy)butyl, 3-(n-butoxy)butyl, 3-(1-methylpropoxy)butyl, 3-(2-methylpropoxy)butyl, 3-(1,1-dimethylethoxy)butyl, 4-(methoxy)butyl, 4-(ethoxy)butyl, 4-(n-propoxy)butyl, 4-(1-methylethoxy)butyl, 4-(n-butoxy)butyl, 4-(1-methylpropoxy)butyl, 4-(2-methylpropoxy)butyl or 4-(1,1-dimethylethoxy)butyl, preferably CH_{2—OCH}₃, CH₂—OC₂H₅, 2-methoxyethyl or 2-ethoxyethyl;

C₁-C₄-alkylthio-C₁-C₄-alkyl: C₁-C₄-alkyl which is substituted by C₁-C₄-alkylthio as mentioned above, i.e. for example CH₂—SCH₃, CH₂—SC₂H₅, n-propylthiomethyl, CH₂—SCH(CH₃)₂, n-butylthiomethyl, (1-methylpropylthio)methyl, (2-methylpropylthio)methyl, CH₂—SC(CH₃)₃, 2-(methylthio)ethyl, 2-(ethylthio)ethyl, 2-(n-propylthio)ethyl, 2-(1-methylethylthio)ethyl, 2-(n-butylthio)ethyl, 2-(1-methylpropylthio)ethyl, 2-(2-methylpropylthio)ethyl, 2-(1,1-dimethylethylthio)ethyl, 2-(methylthio)propyl, 2-(ethylthio)propyl, 2-(n-propylthio)propyl, 2-(1-methylethylthio)propyl, 2-(n-butylthio)propyl, 2-(1-methylpropylthio)propyl, 2-(2-methylpropylthio)propyl, 2-(1,1-dimethylethylthio)propyl, 3-(methylthio)propyl, 3-(ethylthio)propyl, 3-(n-propylthio)propyl, 3-(1-methylethylthio)propyl, 3-(n-butylthio)propyl, 3-(1-methylpropylthio)propyl, 3-(2-methylpropylthio)propyl, 3-(1,1-dimethylethylthio)propyl, 2-(methylthio)butyl, 2-(ethylthio)butyl, 2-(n-propylthio)butyl, 2-(1-methylethylthio)butyl, 2-(n-butylthio)butyl, 2-(1-methylpropylthio)butyl, 2-(2-methylpropylthio)butyl, 2-(1,1-dimethylethylthio)butyl, 3-(methylthio)butyl, 3-(ethylthio)butyl, 3-(n-propylthio)butyl, 3-(1-methylethylthio)butyl, 3-(n-butylthio)butyl, 3-(1-methylpropylthio)butyl, 3-(2-methylpropylthio)butyl, 3-(1,1-dimethylethylthio)butyl, 4-(methylthio)butyl, 4-(ethylthio)butyl, 4-(n-propylthio)butyl, 4-(1-methylethylthio)butyl, 4-(n-butylthio)butyl, 4-(1-methylpropylthio)butyl, 4-(2-methylpropylthio)butyl or 4-(1,1-dimethylethylthio)butyl, preferably CH₂—SCH₃, CH₂—SC₂H₅, 2-methylthioethyl or 2-ethylthioethyl;

(C₁-C₄-alkyl)carbonyl: CO—CH₃, CO—C₂H₅, CO—CH₂—C₂H₅, CO—CH(CH₃)₂, n-butylcarbonyl, CO—CH(CH₃)—C₂H₅, CO—CH₂—CH(CH₃₎₂ or CO—C(CH₃)₃, preferably CO—CH₃ or CO—C₂H₅;

(C₁-C₄-haloalkyl)carbonyl: a (C₁-C₄-alkyl)carbonyl radical as mentioned above which is partially or fully substituted by fluorine, chlorine, bromine and/or iodine, i.e. for example CO—CH₂F, CO—CHF₂, CO—CF₃, CO—CH₂Cl, CO—CH(Cl)₂, CO—C(Cl)₃, chlorofluoromethylcarbonyl, dichlorofluoromethylcarbonyl, chlorodifluoromethylcarbonyl, 2-fluoroethylcarbonyl, 2-chloroethylcarbonyl, 2-bromoethylcarbonyl, 2-iodoethylcarbonyl, 2,2-difluoroethylcarbonyl, 2,2,2-trifluoroethylcarbonyl, 2-chloro-2-fluoroethylcarbonyl, 2-chloro-2,2-difluoroethylcarbonyl, 2,2-dichloro-2-fluoroethylcarbonyl, 2,2,2-trichloroethylcarbonyl, CO—C₂F₅, 2-fluoropropylcarbonyl, 3-fluoropropylcarbonyl, 2,2-difluoropropylcarbonyl, 2,3-difluoropropylcarbonyl, 2-chloropropylcarbonyl, 3-chloropropylcarbonyl, 2,3-dichloropropylcarbonyl, 2-bromopropylcarbonyl, 3-bromopropylcarbonyl, 3,3,3-trifluoropropylcarbonyl, 3,3,3-trichloropropylcarbonyl, 2,2,3,3,3-pentafluoropropylcarbonyl, CO—CF₂-C₂F₅, 1-(CH₂F)-2-fluoroethylcarbonyl, 1-(CH₂Cl)-2-chloroethylcarbonyl, 1-(CH₂Br)-2-bromoethylcarbonyl, 4-fluorobutylcarbonyl, 4-chlorobutylcarbonyl, 4-bromobutylcarbonyl or nonafluorobutylcarbonyl, preferably CO—CF₃, CO—CH₂Cl or 2,2,2-trifluoroethylcarbonyl;

(C₁-C₄-alkyl)carbonyloxy: O—CO—CH₃, O—CO—C₂H₅, O—CO—CH₂—C₂H₅, O—CO—CH(CH₃)₂, O—CO—CH₂—CH₂—C₂H₅, O—CO—CH(CH₃)—C₂H₅, O—CO—CH₂—CH(CH₃)₂ or O—CO—C(CH₃)₃, preferably O—CO—CH₃ or O—CO—C₂H₅;

(C₁-C₄-haloalkyl)carbonyloxy: a (C₁-C₄-alkyl)carbonyl radical as mentioned above which is partially or fully substituted by fluorine, chlorine, bromine and/or iodine, i.e. for example O—CO—CH₂F, O—CO—CHF₂, O—CO—CF₃, O—CO—CH₂Cl, O—CO—CH(Cl)₂, O—CO—C(Cl)₃, chlorofluoromethylcarbonyloxy, dichlorofluoromethylcarbonyloxy, chlorodifluoromethylcarbonyloxy, 2-fluoroethylcarbonyloxy, 2-chloroethylcarbonyloxy, 2-bromoethylcarbonyloxy, 2-iodoethylcarbonyloxy, 2,2-difluoroethylcarbonyloxy, 2,2,2-trifluoroethylcarbonyloxy, 2-chloro-2-fluoroethylcarbonyloxy, 2-chloro-2,2-difluoroethylcarbonyloxy, 2,2-dichloro-2-fluoroethylcarbonyloxy, 2,2,2-trichloroethylcarbonyloxy, O—CO—C₂F₅, 2-fluoropropylcarbonyloxy, 3-fluoropropylcarbonyloxy, 2,2-difluoropropylcarbonyloxy, 2,3-difluoropropylcarbonyloxy, 2-chloropropylcarbonyloxy, 3-chloropropylcarbonyloxy, 2,3-dichloropropylcarbonyloxy, 2-bromopropylcarbonyloxy, 3-bromopropylcarbonyloxy, 3,3,3-trifluoropropylcarbonyloxy, 3,3,3-trichloropropylcarbonyloxy, 2,2,3,3,3-pentafluoropropylcarbonyloxy, heptafluoropropylcarbonyloxy, 1-(CH₂F)-2-fluoroethylcarbonyloxy, 1-(CH₂Cl)-2-chloroethylcarbonyloxy, 1-(CH₂Br)-2-bromoethylcarbonyloxy, 4-fluorobutylcarbonyloxy, 4-chlorobutylcarbonyloxy, 4-bromobutylcarbonyloxy or nonafluorobutylcarbonyloxy, preferably O—CO—CF₃, O—CO—CH₂Cl, or 2,2,2-trifluoroethylcarbonyloxy;

(C₁-C₄-alkoxy)carbonyl: CO—OCH₃, CO—OC₂H₅, n-propoxycarbonyl, CO—OCH(CH₃)₂, n-butoxycarbonyl, CO—OCH(CH₃)—C₃H₅, CO—OCH₂—CH(CH₃)₂ or CO—OC(CH₃)₃, preferably CO—OCH₃ or CO—OC₂H₅;

(C₁-C₄-alkoxy)carbonyl-C₁-C₄-alkyl: C₁-C₄-alkyl which is substituted by (C₁-C₄-alkoxy)carbonyl as mentioned above, i.e. for example methoxycarbonylmethyl, ethoxycarbonylmethyl, n-propoxycarbonylmethyl, (1-methylethoxycarbonyl)methyl, n-butoxycarbonylmethyl, (1-methylpropoxycarbonyl)methyl, (2-methylpropoxycarbonyl)methyl, (1,1-dimethylethoxycarbonyl)methyl, 1-(methoxycarbonyl)ethyl, 1-(ethoxy-carbonyl)etlyl, 1-(n-propoxycarbonyl)ethyl, 1-(1-methylethoxycarbonyl)ethyl, 1-(n-butoxycarbonyl)ethyl, 2-(methoxycarbonyl)ethyl, 2-(ethoxycarbonyl)ethyl, 2-(n-propoxycarbonyl)ethyl, 2-(1-methylethoxycarbonyl)ethyl, 2-(n-butoxycarbonyl)ethyl, 2-(1-methylpropoxycarbonyl)ethyl, 2-(2-methylpropoxycarbonyl)ethyl, 2-(1,1-dimethylethoxycarbonyl)ethyl, 2-(methoxycarbonyl)propyl, 2-(ethoxycarbonyl)propyl, 2-(n-propoxycarbonyl)propyl, 2-(1-methylethoxycarbonyl)propyl, 2-(n-butoxycarbonyl)propyl, 2-(1-methylpropoxycarbonyl)propyl, 2-(2-methylpropoxycarbonyl)propyl, 2-(1,1-dimethylethoxycarbonyl)propyl, 3-(methoxycarbonyl)propyl, 3-(ethoxycarbonyl)propyl, 3-(n-propoxycarbonyl)propyl, 3-(1-methylethoxycarbonyl)propyl, 3-(n-butoxycarbonyl)propyl, 3-(1-methylpropoxycarbonyl)propyl, 3-(2-methylpropoxycarbonyl)propyl, 3-(1,1-dimethylethoxycarbonyl)propyl, 2-(methoxycarbonyl)butyl, 2-(ethoxycarbonyl)butyl, 2-(n-propoxycarbonyl)butyl, 2-(1-methylethoxycarbonyl)butyl, 2-(n-butoxycarbonyl)butyl, 2-(1-methylpropoxycarbonyl)butyl, 2-(2-methylpropoxycarbonyl)butyl, 2-(1,1-dimethylethoxycarbonyl)butyl, 3-(methoxycarbonyl)butyl, 3-(ethoxycarbonyl)butyl, 3-(n-propoxycarbonyl)butyl, 3-(1-methylethoxycarbonyl)butyl, 3-(n-butoxycarbonyl)butyl, 3-(1-methylpropoxycarbonyl)butyl, 3-(2-methylpropoxycarbonyl)butyl, 3-(1,1-dimethylethoxycarbonyl)butyl, 4-(methoxycarbonyl)butyl, 4-(ethoxycarbonyl)butyl, 4-(n-propoxycarbonyl)butyl, 4-(1-methylethoxycarbonyl)butyl, 4-(n-butoxycarbonyl)butyl, 4-(1-methylpropoxycarbonyl)butyl, 4-(2-methylpropoxycarbonyl)butyl or 4-(1,1-dimethylethoxycarbonyl)butyl, preferably methoxycarbonylmethyl, ethoxycarbonylmethyl, 1-(methoxycarbonyl)ethyl or 1-(ethoxycarbonyl)ethyl;

(C₁-C₄-alkoxy)carbonyl-C₁-C₄-alkoxy: C₁-C₄-alkoxy which is substituted by (C₁-C₄-alkoxy)carbonyl as mentioned above, i.e., for example, methoxycarbonylmethoxy, ethoxycarbonylmethoxy, n-propoxycarbonylmethoxy, (1-methylethoxycarbonyl)methoxy, n-butoxycarbonylmethoxy, (1-methylpropoxycarbonyl)methoxy, (2-methylpropoxycarbonyl)methoxy, (1,1-dimethylethoxycarbonyl)methoxy, 1-(methoxycarbonyl)ethoxy, 1-(ethoxycarbonyl)ethoxy, 1-(n-propoxycarbonyl)ethoxy, 1-(1-methylethoxycarbonyl)ethoxy, 1-(n-butoxycarbonyl)ethoxy, 2-(methoxycarbonyl)ethoxy, 2-(ethoxycarbonyl)ethoxy, 2-(n-propoxycarbonyl)ethoxy, 2-(1-methylethoxycarbonyl)ethoxy, 2-(n-butoxycarbonyl)ethoxy, 2-(1-methylpropoxycarbonyl)ethoxy, 2-(2-methylpropoxycarbonyl)ethoxy, 2-(1,1-dimethylethoxycarbonyl)ethoxy, 2-(methoxycarbonyl)propoxy, 2-(ethoxycarbonyl)propoxy, 2-(n-propoxycarbonyl)propoxy, 2-(1-methylethoxycarbonyl)propoxy, 2-(n-butoxycarbonyl)propoxy, 2-(1-methylpropoxycarbonyl)propoxy, 2-(2-methylpropoxycarbonyl)propoxy, 2-(1,1-dimethylethoxycarbonyl)propoxy, 3-(methoxycarbonyl)propoxy, 3-(ethoxycarbonyl)propoxy, 3-(n-propoxycarbonyl)propoxy, 3-(1-methylethoxycarbonyl)propoxy, 3-(n-butoxycarbonyl)propoxy, 3-(1-methylpropoxycarbonyl)propoxy, 3-(2-methylpropoxycarbonyl)propoxy, 3-(1,1-dimethylethoxycarbonyl)propoxy, 2-(methoxycarbonyl)butoxy, 2-(ethoxycarbonyl)butoxy, 2-(n-propoxycarbonyl)butoxy, 2-(1-methylethoxycarbonyl)butoxy, 2-(n-butoxycarbonyl)butoxy, 2-(1-methylpropoxycarbonyl)butoxy, 2-(2-methylpropoxycarbonyl)butoxy, 2-(1,1-dimethylethoxycarbonyl)butoxy, 3-(methoxycarbonyl)butoxy, 3-(ethoxycarbonyl)butoxy, 3-(n-propoxycarbonyl)butoxy, 3-(1-methylethoxycarbonyl)butoxy, 3-(n-butoxycarbonyl)butoxy, 3-(1-methylpropoxycarbonyl)butoxy, 3-(2-methylpropoxycarbonyl)butoxy, 3-(1,1-dimethylethoxycarbonyl)butoxy, 4-(methoxycarbonyl)butoxy, 4-(ethoxycarbonyl)butoxy, 4-(n-propoxycarbonyl)butoxy, 4-(1-methylethoxycarbonyl)butoxy, 4-(n-butoxycarbonyl)butoxy, 4-(1-methylpropoxycarbonyl)butoxy, 4-(2-methylpropoxycarbonyl)butyl or 4-(1,1-dimethylethoxycarbonyl)butoxy, preferably methoxycarbonylmethoxy, ethoxycarbonylmethoxy, 1-(methoxycarbonyl)ethoxy or 1-(ethoxycarbonyl)ethoxy;

(C₁-C₄-alkoxy)carbonyl-C₁-C₄-alkylthio: C₁-C₄-alkylthio which is substituted by (C₁-C₄-alkoxy)carbonyl as mentioned above, i.e., for example, methoxycarbonylmethylthio, ethoxycarbonylmethylthio, n-propoxycarbonylmethylthio, (1-methylethoxycarbonyl)methylthio, n-butoxycarbonylmethylthio, (1-methylpropoxycarbonyl)methylthio, (2-methylpropoxycarbonyl)methylthio, (1,1-dimethylethoxycarbonyl)methylthio, 1-(methoxycarbonyl)ethylthio, 1-(ethoxycarbonyl)ethylthio, 1-(n-propoxycarbonyl)ethylthio, 1-(1-methylethoxycarbonyl)ethylthio, 1-(n-butoxycarbonyl)ethylthio, 2-(methoxycarbonyl)ethylthio, 2-(ethoxycarbonyl)ethylthio, 2-(n-propoxycarbonyl)ethylthio, 2-(1-methylethoxycarbonyl)ethylthio, 2-(n-butoxycarbonyl)ethylthio, 2-(1-methylpropoxycarbonyl)ethylthio, 2-(2-methylpropoxycarbonyl)ethylthio, 2-(1,1-dimethylethoxycarbonyl)ethylthio, 2-(methoxycarbonyl)propylthio, 2-(ethoxycarbonyl)propylthio, 2-(n-propoxycarbonyl)propylthio, 2-(1-methylethoxycarbonyl)propylthio, 2-(n-butoxycarbonyl)propylthio, 2-(1-methylpropoxycarbonyl)propylthio, 2-(2-methylpropoxycarbonyl)propylthio, 2-(1,1-dimethylethoxycarbonyl)propylthio, 3-(methoxycarbonyl)propylthio, 3-(ethoxycarbonyl)propylthio, 3-(n-propoxycarbonyl)propylthio, 3-(1-methylethoxycarbonyl)propylthio, 3-(n-butoxycarbonyl)propylthio, 3-(1-methylpropoxycarbonyl)propylthio, 3-(2-methylpropoxycarbonyl)propylthio, 3-(1,1-dimethylethoxycarbonyl)propylthio, 2-(methoxycarbonyl)butylthio, 2-(ethoxycarbonyl)butylthio, 2-(n-propoxycarbonyl)butylthio, 2-(1-methylethoxycarbonyl)butylthio, 2-(n-butoxycarbonyl)butylthio, 2-(1-methylpropoxycarbonyl)butylthio, 2-(2-methylpropoxycarbonyl)butylthio, 2-(1,1-dimethylethoxycarbonyl)butylthio, 3-(methoxycarbonyl)butylthio, 3-(ethoxycarbonyl)butylthio, 3-(n-propoxycarbonyl)butylthio, 3-(1-methylethoxycarbonyl)butylthio, 3-(n-butoxycarbonyl)butylthio, 3-(1-methylpropoxycarbonyl)butylthio, 3-(2-methylpropoxycarbonyl)butylthio, 3-(1,1-dimethylethoxycarbonyl)butylthio, 4-(methoxycarbonyl)butylthio, 4-(ethoxycarbonyl)butylthio, 4-(n-propoxycarbonyl)butylthio, 4-(1-methylethoxycarbonyl)butylthio, 4-(n-butoxycarbonyl)butylthio, 4-(1-methylpropoxycarbonyl)butylthio, 4-(2-methylpropoxycarbonyl)butyl or 4-(1,1-dimethylethoxycarbonyl)butylthio, preferably methoxycarbonylmethylthio, ethoxycarbonylmethylthio, 1-(methoxycarbonyl)ethylthio or 1-(ethoxycarbonyl)ethylthio;

C₁-C₄-alkylsulfinyl: SO—CH₃, SO—C₂H₅, SO—CH₂—C₂H₅, SO—CH(CH₃)₂, n-butylsulfinyl, SO—CH(CH₃)—C₂H₅, SO—CH₂—CH(CH₃)₂ or SO—C(CH₃)₃, preferably SO—CH₃ or SO—C₂H₅;

C₁-C₄-haloalkylsulfinyl: a C₁-C₄-alkylsulfinyl radical as mentioned above which ispartially or fully substituted by fluorine, chlorine, bromine and/or iodine, i.e. for example SO—CH₂F, SO—CHF₂, SO—CF₃, SO—CH₂Cl, SO—CH(Cl)₂, SO—C(Cl)₃, chlorofluoromethylsulfinyl, dichlorofluoromethylsulfinyl, chlorodifluoromethylsulfinyl, 2-fluoroethylsulfinyl, 2-chloroethylsulfinyl, 2-bromoethylsulfinyl, 2-iodoethylsulfinyl, 2,2-difluoroethylsulfinyl, 2,2,2-trifluoroethylsulfinyl, 2-chloro-2-fluoroethylsulfinyl, 2-chloro-2,2-difluoroethylsulfinyl, 2,2-dichloro-2-fluoroethylsulfinyl, 2,2,2-trichloroethylsulfinyl, SO—C₂F₅, 2-fluoropropylsulfinyl, 3-fluoropropylsulfinyl, 2,2-difluoropropylsulfinyl, 2,3-difluoropropylsulfinyl, 2-chloropropylsulfinyl, 3-chloropropylsulfinyl, 2,3-dichloropropylsulfinyl, 2-bromopropylsulfinyl, 3-bromopropylsulfinyl, 3,3,3-trifluoropropylsulfinyl, 3,3,3-trichloropropylsulfinyl, SO—CH₂—C₂F₅, SO—CF₂—C₂F₅, 1-(fluoromethyl)-2-fluoroethylsulfinyl, 1-(chloromethyl)-2-chloroethylsulfinyl, 1-(bromomethyl)-2-bromoethylsulfinyl, 4-fluorobutylsulfinyl, 4-chlorobutylsulfinyl, 4-bromobutylsulfinyl or nonafluorobutylsulfinyl, preferably SO—CF₃, SO—CH₂Cl or 2,2,2-trifluoroethylsulfinyl;

C₁-C₄-alkylsulfonyl: SO₂—CH₃, SO₂—C₂H₅, SO₂—CH₂—C₂H₅, SO₂—CH(CH₃)₂, n-butylsulfonyl, SO₂—CH(CH₃)—C₂H₅, SO₂—CH₂—CH(CH₃)₂ or SO₂—C(CH₃)₃, preferably SO₂—CH₃ or SO₂—C₂H₅;

C₁-C₄-haloalkylsulfonyl: a C₁-C₄-alkylsulfonyl radical as mentioned above which is partially or fully substituted by fluorine, chlorine, bromine and/or iodine, i.e. for example SO₂—CH₂F, SO₂—CHF₂, SO₂—CF₃, SO₂—CH₂Cl, SO₂—CH(Cl)₂, SO_{2—C(Cl)}₃, chlorofluoromethylsulfonyl, dichlorofluoromethylsulfonyl, chlorodifluoromethylsulfonyl, 2-fluoroethylsulfonyl, 2-chloroethylsulfonyl, 2-bromoethylsulfonyl, 2-iodoethylsulfonyl, 2,2-difluoroethylsulfonyl, 2,2,2-trifluoroethylsulfonyl, 2-chloro-2-fluoroethylsulfonyl, 2-chloro-2,2-difluoroethylsulfonyl, 2,2-dichloro-2-fluoroethylsulfonyl, 2,2,2-trichloroethylsulfonyl, SO₂—C₂F₅, 2-fluoropropylsulfonyl, 3-fluoropropylsulfonyl, 2,2-difluoropropylsulfonyl, 2,3-difluoropropylsulfonyl, 2-chloropropylsulfonyl, 3-chloropropylsulfonyl, 2,3-dichloropropylsulfonyl, 2-bromopropylsulfonyl, 3-bromopropylsulfonyl, 3,3,3-trifluoropropylsulfonyl, 3,3,3-trichloropropylsulfonyl, SO₂—CH₂—C₂F₅, SO₂—CF₂—C₂F₅, 1-(fluoromethyl)-2-fluoroethylsulfonyl, 1-(chloromethyl)-2-chloroethylsulfonyl, 1-(bromomethyl)-2-bromoethylsulfonyl, 4-fluorobutylsulfonyl, 4-chlorobutylsulfonyl, 4-bromobutylsulfonyl or nonafluorobutylsulfonyl, preferably SO₂—CF₃, SO₂—CH₂Cl or 2,2,2-trifluoroethylsulfonyl;

di(C₁-C₄-alkyl)amino: N(CH₃)₂, N(C₂H₅), N,N-dipropylamino, N[CH(CH₃)₂]₂, N,N-dibutylamino, N,N-di(1-methylpropyl)amino, N,N-di(2-methylpropyl)amino, N[C(CH₃)₃]₂, N-ethyl-N-methylamino, N-methyl-N-propylamino, N-methyl-N-(1-methylethyl)amino, N-butyl-N-methylamino, N-methyl-N-(1-methylpropyl)amino, N-methyl-N-(2-methylpropyl)amino, N-(1,1-dimethylethyl)-N-methylamino, N-ethyl-N-propylamino, N-ethyl-N-(1-methylethyl)amino, N-butyl-N-ethylamino, N-ethyl-N-(1-methylpropyl)amino, N-ethyl-N-(2-methylpropyl)amino, N-ethyl-N-(1,1-dimethylethyl)amino, N-(1-methylethyl)-N-propylamino, N-butyl-N-propylamino, N-(1-methylpropyl)-N-propylamino, N-(2-methylpropyl)-N-propylamino, N-(1,1-dimethylethyl)-N-propylamino, N-butyl-N-(1-methylethyl)amino, N-(1-methylethyl)-N-(1-methylpropyl)amino, N-(1-methylethyl)-N-(2-methylpropyl)amino, N-(1,1-dimethylethyl)-N-(1-methylethyl)amino, N-butyl-N-(1-methylpropyl)amino, N-butyl-N-(2-methylpropyl)amino, N-butyl-N-(1,1-dimethylethyl)amino, N-(1-methylpropyl)-N-(2-methylpropyl)amino, N-(1,1-dimethylethyl)-N-(1-methylpropyl)amino or N-(1,1-dimethylethyl)-N-(2-methylpropyl)amino, preferably N(CH₃)₂ or N(C₂H₅);

di(C₁-C₄-alkyl)aminocarbonyl: for example N,N-dimethylaminocarbonyl, N,N-diethylaminocarbonyl, N,N-di(1-methylethyl)aminocarbonyl, N,N-dipropylaminocarbonyl, N,N-dibutylaminocarbonyl, N,N-di(1-methylpropyl)aminocarbonyl, N,N-di(2-methylpropyl)aminocarbonyl, N,N-di(1,1-dimethylethyl)aminocarbonyl, N-ethyl-N-methylaminocarbonyl, N-methyl-N-propylaminocarbonyl, N-methyl-N-(1-methylethyl)aminocarbonyl, N-butyl-N-methylaminocarbonyl, N-methyl-N-(1-methylpropyl)aminocarbonyl, N-methyl-N-(2-methylpropyl)aminocarbonyl, N-(1,1-dimethylethyl)-N-methylaminocarbonyl, N-ethyl-N-propylaminocarbonyl, N-ethyl-N-(1-methylethyl)aminocarbonyl, N-butyl-N-ethylaminocarbonyl, N-ethyl-N-(1-methylpropyl)aminocarbonyl, N-ethyl-N-(2-methylpropyl)aminocarbonyl, N-ethyl-N-(1,1-dimethylethyl)aminocarbonyl, N-(1-methylethyl)-N-propylaminocarbonyl, N-butyl-N-propylaminocarbonyl, N-(1-methylpropyl)-N-propylaminocarbonyl, N-(2-methylpropyl)-N-propylaminocarbonyl, N-(1,1-dimethylethyl)-N-propylaminocarbonyl, N-butyl-N(1-methylethyl)aminocarbonyl, N-(1-methylethyl)-N-(1-methylpropyl)aminocarbonyl, N-(1-methylethyl)-N-(2-methylpropyl)aminocarbonyl, N-(1,1-dimethylethyl)-N-(1-methylethyl)aminocarbonyl, N-butyl-N-(1-methylpropyl)aminocarbonyl, N-butyl-N-(2-methylpropyl)aminocarbonyl, N-butyl-N-(1,1-dimethylethyl)aminocarbonyl, N-(1-methylpropyl)-N-(2-methylpropyl)aminocarbonyl, N-(1,1-dimethylethyl)-N-(1-methylpropyl)aminocarbonyl or N-(1,1-dimethylethyl)-N-(2-methylpropyl)aminocarbonyl;

di(C₁-C₄-alkyl)aminocarbonyl-C₁-C₄-alkyl: C₁-C₄-alkyl which is monosubstituted by di(C₁-C₄-alkyl)aminocarbonyl, for example di(C₁-C₄-alkyl)aminocarbonylmethyl, 1- or 2-di(C₁-C₄-alkyl)aminocarbonylethyl, 1-, 2- or 3-di(C₁-C₄-alkyl)aminocarbonylpropyl;

di(C₁-C₄-alkyl)aminocarbonyl-C₁-C₄-alkoxy: C₁-C₄-alkoxy which is monosubstituted by di(C₁-C₄-alkyl)aminocarbonyl, for example di(C₁-C₄-alkyl)aminocarbonylmethoxy, 1- or 2-di(C₁-C₄-alkyl)aminocarbonylethoxy, 1-, 2- or 3-di(C₁-C₄-alkyl)aminocarbonylpropoxy;

di(C₁-C₄-alkyl)aminocarbonyl-C₁-C₄-alkylthio: C₁-C₄-alkylthio which is monosubstituted by di(C₁-C₄-alkyl)aminocarbonyl, for example di(C₁-C₄-alkyl)aminocarbonylmethylthio, 1- or 2-di(C₁-C₄-alkyl)aminocarbonylethylthio, 1-, 2- or 3-di(C₁-C₄-alkyl)aminocarbonylpropylthio;

C₂-C₆-alkenyl: vinyl, prop-1-en-1-yl, allyl, 1-methylethenyl, 1-buten-1-yl, 1-buten-2-yl, 1-buten-3-yl, 2-buten-1-yl, 1-methylprop-1-en-1-yl, 2-methylprop-1-en-1-yl, 1-methyl-prop-2-en-1-yl, 2-methylprop-2-en-1-yl, n-penten-1-yl, n-penten-2-yl, n-penten-3-yl, n-penten-4-yl, 1-methylbut-1-en-1-yl, 2-methylbut-1-en-1-yl, 3-methylbut-1-en-1-yl, 1-methylbut-2-en-1-yl, 2-methylbut-2-en-1-yl, 3-methylbut-2-en-1-yl, 1-methylbut-3-en-1-yl, 2-methylbut-3-en-1-yl, 3-methylbut-3-en-1-yl, 1,1-dimethylprop-2-en-1-yl, 1,2-dimethylprop-1-en-1-yl, 1,2-dimethylprop-2-en-1-yl, 1-ethylprop-1-en-2-yl, 1-ethylprop-2-en-1-yl, n-hex-1-en-1-yl, n-hex-2-en-1-yl, n-hex-3-en-1-yl, n-hex-4-en-1-yl, n-hex-5-en-1-yl, 1-methylpent-1-en-1-yl, 2-methylpent-1-en-1-yl, 3-methylpent-1-en-1-yl, 4-methylpent-1-en-1-yl, 1-methylpent-2-en-1-yl, 2-methylpent-2-en-1-yl, 3-methylpent-2-en-1-yl, 4-methylpent-2-en-1-yl, 1-methylpent-3-en-1-yl, 2-methylpent-3-en-1-yl, 3-methylpent-3-en-1-yl, 4-methylpent-3-en-1-yl, 1-methylpent-4-en-1-yl, 2-methylpent-4-en-1-yl, 3-methylpent-4-en-1-yl, 4-methylpent-4-en-1-yl, 1,1-dimethylbut-2-en-1-yl, 1,1-dimethylbut-3-en-1-yl, 1,2-dimethylbut-1-en-1-yl, 1,2-dimethylbut-2-en-1-yl, 1,2-dimethylbut-3-en-1-yl, 1,3-dimethylbut-1-en-1-yl, 1,3-dimethylbut-2-en-1-yl, 1,3-dimethylbut-3-en-1-yl, 2,2-dimethylbut-3-en-1-yl, 2,3-dimethylbut-1-en-1-yl, 2,3-dimethylbut-2-en-1-yl, 2,3-dimethylbut-3-en-1-yl, 3,3-dimethylbut-1-en-1-yl, 3,3-dimethylbut-2-en-1-yl, 1-ethylbut-1-en-1-yl, 1-ethylbut-2-en-1-yl, 1-ethylbut-3-en-1-yl, 2-ethylbut-1-en-1-yl, 2-ethylbut-2-en-1-yl, 2-ethylbut-3-en-1-yl, 1,1,2-trimethylprop-2-en-1-yl, 1-ethyl-1-methylprop-2-en-1-yl, 1-ethyl-2-methylprop-1-en-1-yl or 1-ethyl-2-methylprop-2-en-1-yl;

C₂-C₆-haloalkenyl: C₂-C₆-alkenyl as mentioned above which is partially or fully substituted by fluorine, chlorine and/or bromine, i.e. for example 2-chlorovinyl, 2-chloroallyl, 3-chloroallyl, 2,3-dichloroallyl, 3,3-dichloroallyl, 2,3,3-trichloroallyl, 2,3-dichlorobut-2-enyl, 2-bromoallyl, 3-bromoallyl, 2,3-dibromoallyl, 3,3-dibromoallyl, 2,3,3-tribromoallyl and 2,3-dibromobut-2-enyl, preferably C₃- or C₄-haloalkenyl;

C₂-C₆-alkynyl: ethynyl and C₃-C₆-alkynyl, such as prop-1-yn-1-yl, prop-2-yn-1-yl, n-but-1-yn-1-yl, n-but-1-yn-3-yl, n-but-1-yn-4-yl, n-but-2-yn-1-yl, n-pent-1-yn-1-yl, n-pent-1-yn-3-yl, n-pent-1-yn-4-yl, n-pent-1-yn-5-yl, n-pent-2-yn-1-yl, n-pent-2-yn-4-yl, n-pent-2-yn-5-yl, 3-methylbut-1-yn-3-yl, 3-methylbut-1-yn-4-yl, n-hex-1-yn-1-yl, n-hex-1-yn-3-yl, n-hex-1-yn-4-yl, n-hex-1-yn-5-yl, n-hex-1-yn-6-yl, n-hex-2-yn-1-yl, n-hex-2-yn-4-yl, n-hex-2-yn-5-yl, n-hex-2-yn-6-yl, n-hex-3-yn-1-yl, n-hex-3-yn-2-yl, 3-methylpent-1-yn-1-yl, 3-methylpent-1-yn-3-yl, 3-methylpent-1-yn-4-yl, 3-methylpent-1-yn-5-yl, 4-methylpent-1-yn-1-yl, 4-methylpent-2-yn-4-yl or 4-methylpent-2-yn-5-yl, preferably prop-2-yn-1-yl;

C₂-C₆-haloalkynyl: C₂-C₆-alkynyl as mentioned above which is partially or fully substituted by fluorine, chlorine and/or bromine, i.e. for example 1,1-difluoroprop-2-yn-1-yl, 1,1-difluorobut-2-yn-1-yl, 4-fluorobut-2-yn-1-yl, 4-chlorobut-2-yn-1-yl, 5-fluoropent-3-yn-1-yl or 6-fluorohex-4-yn-1-yl, preferably C₃- or C₄-haloalkynyl;

C₃-C₈-cycloalkyl: cyclopropyl, cyclobutyl, cyclopentyl, cyclohexyl, cycloheptyl or cyclooctyl;

C₃-C₈-cycloalkyl containing a carbonyl or thiocarbonyl ring member: for example cyclobutanon-2-yl, cyclobutanon-3-yl, cyclopentanon-2-yl, cyclopentanon-3-yl, cyclohexanon-2-yl, cyclohexanon-4-yl, cycloheptanon-2-yl, cyclooctanon-2-yl, cyclobutanethion-2-yl, cyclobutanethion-3-yl, cyclopentanethion-2-yl, cyclopentanethion-3-yl, cyclohexanethion-2-yl, cyclohexanethion-4-yl, cycloheptanethion-2-yl or cyclooctanethion-2-yl, preferably cyclopentanon-2-yl or cyclohexanon-2-yl;

C₃-C₈-cycloalkyl-C₁-C₄-alkyl: cyclopropylmethyl, 1-cyclopropylethyl, 2-cyclopropylethyl, 1-cyclopropylprop-1-yl, 2-cyclopropylprop-1-yl, 3-cyclopropylprop-1-yl, 1-cyclopropylbut-1-yl, 2-cyclopropylbut-1-yl, 3-cyclopropylbut-1-yl, 4-cyclopropylbut-1-yl, 1-cyclopropylbut-2-yl, 2-cyclopropylbut-2-yl, 3-cyclopropylbut-2-yl, 4-cyclopropylbut-2-yl, 1-(cyclopropylmethyl)eth-1-yl, 1-(cyclopropylmethyl)-1-(methyl)eth-1-yl, 1-(cyclopropylmethyl)prop-1-yl, cyclobutylmethyl, 1-cyclobutylethyl, 2-cyclobutylethyl, 1-cyclobutylprop-1-yl, 2-cyclobutylprop-1-yl, 3-cyclobutylprop-1-yl, 1-cyclobutylbut-1-yl, 2-cyclobutylbut-1-yl, 3-cyclobutylbut-1-yl, 4-cyclobutylbut-1-yl, 1-cyclobutylbut-2-yl, 2-cyclobutylbut-2-yl, 3-cyclobutylbut-2-yl, 4-cyclobutylbut-2-yl, 1-(cyclobutylmethyl)eth-1-yl, 1-(cyclobutylmethyl)-1-(methyl)eth-1-yl, 1-(cyclobutylmethyl)prop-1-yl, cyclopentylmethyl, 1-cyclopentylethyl, 2-cyclopentylethyl, 1-cyclopentylprop-1-yl, 2-cyclopentylprop-1-yl, 3-cyclopentylprop-1-yl, 1-cyclopentylbut-1-yl, 2-cyclopentylbut-1-yl, 3-cyclopentylbut-1-yl, 4-cyclopentylbut-1-yl, 1-cyclopentylbut-2-yl, 2-cyclopentylbut-2-yl, 3-cyclopentylbut-2-yl, 4-cyclopentylbut-2-yl, 1-(cyclopentylmethyl)eth-1-yl, 1-(cyclopentylmethyl)-1-(methyl)eth-1-yl, 1-(cyclopentylmethyl)prop-1-yl, cyclohexylmethyl, 1-cyclohexylethyl, 2-cyclohexylethyl, 1-cyclohexylprop-1-yl, 2-cyclohexylprop-1-yl, 3-cyclohexyl-prop-1-yl, 1-cyclohexylbut-1-yl, 2-cyclohexylbut-1-yl, 3-cyclohexylbut-1-yl, 4-cyclohexylbut-1-yl, 1-cyclohexylbut-2-yl, 2-cyclohexylbut-2-yl, 3-cyclohexylbut-2-yl, 4-cyclohexylbut-2-yl, 1-(cyclohexylmethyl)eth-1-yl, 1-(cyclohexylmethyl)-1-(methyl)eth-1-yl, 1-(cyclohexylmethyl)prop-1-yl, cycloheptylmethyl, 1-cycloheptylethyl, 2-cycloheptylethyl, 1-cycloheptylprop-1-yl, 2-cycloheptylprop-1-yl, 3-cycloheptylprop-1-yl, 1-cycloheptylbut-1-yl, 2-cycloheptylbut-1-yl, 3-cycloheptylbut-1-yl, 4-cycloheptylbut-1-yl, 1-cycloheptylbut-2-yl, 2-cycloheptylbut-2-yl, 3-cycloheptyl-but-2-yl, 4-cycloheptylbut-2-yl, 1-(cycloheptylmethyl)eth-1-yl, 1-(cycloheptylmethyl)-1-(methyl)eth-1-yl, 1-(cycloheptylmethyl)prop-1-yl, cyclooctylmethyl, 1-cyclooctylethyl, 2-cyclooctylethyl, 1-cyclooctylprop-1-yl, 2-cyclooctylprop-1-yl, 3-cyclooctylprop-1-yl, 1-cyclooctylbut-1-yl, 2-cyclooctylbut-1-yl, 3-cyclooctylbut-1-yl, 4-cyclooctylbut-1-yl, 1-cyclooctylbut-2-yl, 2-cyclooctylbut-2-yl, 3-cyclooctylbut-2-yl, 4-cyclooctyl-but-2-yl, 1-(cyclooctylmethyl)eth-1-yl, 1-(cyclooctylmethyl)-1-(methyl)eth-1-yl or 1-(cyclooctylmethyl)prop-1-yl, preferably cyclopropylmethyl, cyclobutylmethyl, cyclopentylmethyl or cyclohexylmethyl;

C₃-C₈-cycloalkyl-C₁-C₄-alkyl containing a carbonyl or thiocarbonyl ring member: for example cyclobutanon-2-yl-methyl, cyclobutanon-3-ylmethyl, cyclopentanon-2-ylmethyl, cyclopentanon-3-ylmethyl, cyclohexanon-2-ylmethyl, cyclohexanon-4-ylmethyl, cycloheptanon-2-ylmethyl, cyclooctanon-2-ylmethyl, cyclobutanethion-2-ylmethyl, cyclobutanethion-3-ylmethyl, cyclopentanethion-2-ylmethyl, cyclopentanethion-3-ylmethyl, cyclohexanethion-2-ylmethyl, cyclohexanethion-4-ylmethyl, cycloheptanethion-2-ylmethyl, cyclooctanethion-2-ylmethyl, 1-(cyclobutanon-2-yl)ethyl, 1-(cyclobutanon-3-yl)ethyl, 1-(cyclopentanon-2-yl)ethyl, 1-(cyclopentanon-3-yl)ethyl, 1-(cyclohexanon-2-yl)ethyl, 1-(cyclohexanon-4-yl)ethyl, 1-(cycloheptanon-2-yl)ethyl, 1-(cyclooctanon-2-yl)ethyl, 1-(cyclobutanethion-2-yl)ethyl, 1-(cyclobutanethion-3-yl)ethyl, 1-(cyclopentanethion-2-yl)ethyl, 1-(cyclopentanethion-3-yl)ethyl, 1-(cyclohexanethion-2-yl)ethyl, 1-(cyclohexanethion-4-yl)ethyl, 1-(cycloheptanethion-2-yl)ethyl, 1-(cyclooctanethion-2-yl)ethyl, 2-(cyclobutanon-2-yl)ethyl, 2-(cyclobutanon-3-yl)ethyl, 2-(cyclopentanon-2-yl)ethyl, 2-(cyclopentanon-3-yl)ethyl, 2-(cyclohexanon-2-yl)ethyl, 2-(cyclohexanon-4-yl)ethyl, 2-(cycloheptanon-2-yl)ethyl, 2-(cyclooctanon-2-yl)ethyl, 2-(cyclobutanethion-2-yl)ethyl, 2-(cyclobutanethion3-yl)ethyl, 2-(cyclopentanethion-2-yl)ethyl, 2-(cyclopentanethion-3-yl)ethyl, 2-(cyclohexanethion2-yl)ethyl, 2-(cyclohexanethion-4-yl )ethyl, 2-(cycloheptanethion-2-yl)ethyl, 2-(cyclooctanethion-2-yl)ethyl, 3-(cyclobutanon-2-yl)propyl, 3-(cyclobutanon-3-yl)propyl, 3-(cyclopentanon-2-yl)propyl, 3-(cyclopentanon-3-yl)propyl, 3-(cyclohexanon-2-yl)propyl, 3-(cyclohexanon-4-yl)propyl, 3-(cycloheptanon-2-yl)propyl, 3-(cyclooctanon-2-yl)propyl, 3-(cyclobutanethion-2-yl)propyl, 3-(cyclobutanethion-3-yl)propyl, 3-(cyclopentanethion-2-yl)-propyl, 3-(cyclopentanethion-3-yl)propyl, 3-(cyclohexanethion-2-yl)propyl, 3-(cyclohexanethion-4-yl)propyl, 3-(cycloheptanethion-2-yl)propyl, 3-(cyclooctanethion-2-yl)propyl, 4-(cyclobutanon-2-yl)butyl, 4-(cyclobutanon-3-yl)butyl, 4-(cyclopentanon-2-yl)butyl, 4-(cyclopentanon-3-yl)butyl, 4-(cyclohexanon-2-yl)butyl, 4-(cyclohexanon-4-yl)butyl, 4-(cycloheptanon-2-yl)butyl, 4-(cyclooctanon-2-yl)butyl, 4-(cyclobutanethion-2-yl)butyl, 4-(cyclobutanethion-3-yl)butyl, 4-(cyclopentanethion-2-yl)butyl, 4-(cyclopentanethion-3-yl)butyl, 4-(cyclohexanethion-2-yl)butyl, 4-(cyclohexanethion-4-yl)butyl, 4-(cycloheptanethion-2-yl)butyl or 4-(cyclooctanethion-2-yl)butyl, preferably cyclopentanon-2-ylmethyl, cyclohexanon-2-ylmethyl, 2-(cyclopentanon-2-yl)ethyl or 2-(cyclohexanon-2-yl)ethyl.

3- to 7-membered heterocyclyl is a saturated, partially or fully unsaturated or aromatic heterocycle having one, two or three heteroatoms selected from a group consisting of nitrogen atoms, oxygen and sulfur atoms. Saturated 3- to 7-membered heterocyclyl may also contain a carbonyl or thiocarbonyl ring member.

Examples of saturated heterocycles containing a carbonyl or thiocarbonyl ring member are:

oxiranyl, thiiranyl, aziridin-1-yl, aziridin-2-yl, diaziridin-1-yl, diaziridin-3-yl, oxetan-2-yl, oxetan-3-yl, thietan-2-yl, thietan-3-yl, azetidin-1-yl, azetidin-2-yl, azetidin-3-yl, tetrahydrofuran-2-yl, tetrahydrofuran-3-yl, tetrahydrothiophen-2-yl, tetrahydrothiophen-3-yl, pyrrolidin-1-yl, pyrrolidin-2-yl, pyrrolidin-3-yl, 1,3-dioxolan-2-yl, 1,3-dioxolan-4-yl, 1,3-oxathiolan-2-yl, 1,3-oxathiolan-4-yl, 1,3-oxathiolan-5-yl, 1,3-oxazolidin-2-yl, 1,3-oxazolidin-3-yl, 1,3-oxazolidin-4-yl, 1,3-oxazolidin-5-yl, 1,2-oxazolidin-2-yl, 1,2-oxazolidin-3-yl, 1,2-oxazolidin-4-yl, 1,2-oxazolidin-5-yl, 1,3-dithiolan-2-yl, 1,3-dithiolan-4-yl, pyrrolidin-1-yl, pyrrolidin-2-yl, pyrrolidin-5-yl, tetrahydropyrazol-1-yl, tetrahydropyrazol-3-yl, tetrahydropyrazol-4-yl, tetrahydro-pyran-2-yl, tetrahydropyran-3-yl, tetrahydropyran-4-yl, tetrahydrothiopyran-2-yl, tetrahydrothiopyran-3-yl, tetrahydropyran-4-yl, piperidin-1-yl, piperidin-2-yl, piperidin-3-yl, piperidin-4-yl, 1,3-dioxan-2-yl, 1,3-dioxan-4-yl, 1,3-dioxan-5-yl, 1,4-dioxan-2-yl, 1,3-oxathian-2-yl, 1,3-oxathian-4-yl, 1,3-oxathian-5-yl, 1,3-oxathian-6-yl, 1,4-oxathian-2-yl, 1,4-oxathian-3-yl, morpholin-2-yl, morpholin-3-yl, morpholin-4-yl, hexahydropyridazin-1-yl, hexahydropyridazin-3-yl, hexahydropyridazin-4-yl, hexahydropyrimidin-1-yl, hexahydropyrimidin-2-yl, hexahydropyrimidin-4-yl, hexahydropyrimidin-5-yl, piperazin-1-yl, piperazin-2-yl, piperazin-3-yl, hexahydro-1,3,5-triazin-1-yl, hexahydro-1,3,5-triazin-2-yl, oxepan-2-yl, oxepan-3-yl, oxepan-4-yl, thiepan-2-yl, thiepan-3-yl, thiepan-4-yl, 1,3-dioxepan-2-yl, 1,3-dioxepan-4-yl, 1,3-dioxepan-5-yl, 1,3-dioxepan-6-yl, 1,3-dithiepan-2-yl, 1,3-dithiepan-4-yl, 1,3-dithiepan-5-yl, 1,3-dithiepan-6-yl, 1,4-dioxepan-2-yl, 1,4-dioxepan-7-yl, hexahydroazepin-1-yl, hexahydroazepin-2-yl, hexahydroazepin-3-yl, hexahydroazepin-4-yl, hexahydro-1,3-diazepin-1-yl, hexahydro-1,3-diazepin-2-yl, hexahydro-1,3-diazepin-4-yl, hexahydro-1,4-diazepin-1-yl and hexahydro-1,4-diazepin-2-yl.

Examples of unsaturated heterocycles containing a carbonyl or thiocarbonyl ring member are:

dihydrofuran-2-yl, 1,2-oxazolin-3-yl, 1,2-oxazolin-5-yl, 1,3-oxazolin-2-yl.

Examples of aromatic heterocyclyl are the 5- and 6-membered aromatic heterocyclic radicals, for example, furyl, such as 2-furyl and 3-furyl, thienyl, such as 2-thienyl and 3-thienyl, pyrrolyl, such as 2-pyrrolyl and 3-pyrrolyl, isoxazolyl, such as 3-isoxazolyl, 4-isoxazolyl and 5-isoxazolyl, isothiazolyl, such as 3-isothiazolyl, 4-isothiazolyl and 5-isothiazolyl, pyrazolyl, such as 3-pyrazolyl, 4-pyrazolyl and 5-pyrazolyl, oxazolyl, such as 2-oxazolyl, 4-oxazolyl and 5-oxazolyl, thiazolyl, such as 2-thiazolyl, 4-thiazolyl and 5-thiazolyl, imidazolyl, such as 2-imidazolyl and 4-imidazolyl, oxadiazolyl, such as 1,2,4-oxadiazol-3-yl, 1,2,4-oxadiazol-5-yl and 1,3,4-oxadiazol-2-yl, thiadiazolyl, such as 1,2,4-thiadiazol-3-yl, 1,2,4-thiadiazol-5-yl and 1,3,4-thiadiazol-2-yl, triazolyl, such as 1,2,4-triazol-1-yl, 1,2,4-triazol-3-yl and 1,2,4-triazol-4-yl, pyridinyl, such as 2-pyridinyl, 3-pyridinyl and 4-pyridinyl, pyridazinyl, such as 3-pyridazinyl and 4-pyridazinyl, pyrimidinyl, such as 2-pyrimidinyl, 4-pyrimidinyl and 5-pyrimidinyl, and furthermore 2-pyrazinyl, 1,3,5-triazin-2-yl and 1,2,4-triazin-3-yl, in particular pyridyl, pyrimidyl, furanyl and thienyl.

Examples of fused rings are, in addition to phenyl, the abovementioned heteroaromatic groups, in particular pyridine, pyrazine, pyridazine, pyrimidine, furan, dihydrofuran, thiophene, dihydrothiophene, pyrrole, dihydropyrrole, 1,3-dioxolane, 1,3-dioxolan-2-one, isoxazole, oxazole, oxazoline, isothiazole, thiazole, pyrazole, pyrazoline, imidazole, imidazolinone, dihydroimidazole, 1,2,3-triazole, 1,1-dioxodihydroisothiazole, dihydro-1,4-dioxine, pyridone, dihydro-1,4-oxazine, dihydro-1,4-oxazin-2-one, dihydro-1,4-oxazin-3-one, dihydro-1,3-oxazine, dihydro-1,3-thiazin-2-one, dihydro-1,4-thiazine, dihydro-1,4-thiazin-2-one, dihydro-1,4-thiazin-3-one, dihydro-1,3-thiazine and dihydro-1,3-thiazin-2-one, which for their part may have one, two or three substituents. Examples of suitable substituents on the fused ring are the meanings given below for R¹⁶, R¹⁷, R¹⁸ and R¹⁹.

With respect to the use of the 1-aryl-4-haloalkyl-2-[1H]pyridones I as herbicides or desiccants/defoliants, preference is given to the compounds I in which the variables are as defined below, in each case on their own or in combination:

R¹ is hydrogen or halogen, in particular chlorine;

R², R^{2′} independently of one another are hydrogen or C₁-C₄-alkyl, for example methyl;

R³ is C₁-C₄-haloalkyl, in particular C₁-C₂-alkyl which carries, as halogen atoms, chlorine and/or fluorine, particularly preferably trifluoromethyl;

R⁴ is halogen, in particular fluorine or chlorine, or hydrogen;

R⁵ is halogen, in particular chlorine, or cyano;

A is oxygen;

X is a chemical bond, methylene, ethane-1,2-diyl, ethene-1,2-diyl which may be unsubstituted or may have one substituent selected from the group consisting of C₁-C₄-alkyl, especially methyl, or halogen, especially chlorine, for example 1- or 2-chloroethane-1,2-diyl, 1- or 2-chloroethene-1,2-diyl, 1- or 2-bromoethane-1,2-diyl, 1- or 2-bromoethene-1,2-diyl, 1- or 2-methylethane-1,2-diyl, 1- or 2-methylethene-1,2-diyl, in particular a chemical bond, 1- or 2-chloroethane-1,2-diyl, 1- or 2-chloroethene-1,2-diyl, 1- or 2-bromoethene-1,2-diyl, 1- or 2-methylethene-1,2-diyl. If X is substituted ethane-1,2-diyl or ethene-1,2-diyl, the substituent is preferably located at the carbon atom adjacent to the group R⁶;

R⁶ is hydrogen, nitro, halogen, chlorosulfonyl, —O—Y—R⁸, —O—CO—Y—R⁸, —N(Y—R⁸)(Z—R⁹), —N(Y—R⁸)—SO₂—Z—R⁹, —N(SO₂—Y—R⁸)(SO₂—Z—R⁹), —S(O)ₙ—Y—R⁸ where n=0, 1 or 2, —SO₂—O—Y—R⁸, —SO₂—N(Y—R⁸)(Z—R⁹), —C(═NOR¹⁰)—Y—R⁸, —C(═NOR¹⁰)—O—Y—R⁸, —CO—Y—R⁸, —CO—O—Y—R⁸, —CO—S—Y—R⁸, —PO(O—Y—R⁸), —CO—N(Y—R⁸)(Z—R⁹) or —CO—N(Y—R⁸)(O—Z—R⁹), in particular —O—Y—R⁸, —N(Y—R⁸)—SO₂—Z—R⁹, —SO₂—N(Y—R⁸)(Z—R⁹), —C(═NOR¹⁰)—Y—R⁸, —CO—O—Y—R⁸ or —CO—N(Y—R⁸)(Z—R⁹).

The variables R⁸, R⁹, R¹⁰, Y and Z mentioned in the definition of the variable R⁶ are preferably as defined below:

Y, Z independently of one another are a chemical bond or methylene;

R⁸, R⁹ independently of one another are

hydrogen, C₁-C₆-haloalkyl, C₁-C₄-alkoxy-C₁-C₄-alkyl, C₂-C₆-alkenyl, C₂-C₆-haloalkenyl, C₂-C₆-alkynyl, C₂-C₆-haloalkynyl, —CH(R¹¹)(R¹²), —C(R¹¹)(R¹²)—CN, —C(R¹¹)(R¹²)-halogen, —C(R¹¹)(R¹²)—OR¹³, —C(R¹¹)(R¹²)—N(R¹³)R¹⁴, —C(R¹¹)(R¹²)—N(R¹³)—OR¹⁴, —C(R¹¹)(R¹²)—SR¹³, —C(R¹¹)(R¹²)—SO—R¹³, —C(R¹¹)(R¹²)—SO₂—R¹³, —C(R¹¹)(R¹²)—SO₂—OR¹³, —C(R¹¹)(R¹²)—SO₂—N(R¹³)R¹⁴, —C(R¹¹)(R¹²)—CO—R¹³, —C(R¹¹)(R¹²)—C(═NOR¹⁵)—R¹³, —C(R¹¹)(R¹²)—CO—OR¹³, —C(R¹¹)(R¹²)—CO—SR¹³, —C(R¹¹)(R¹²)—CO—N(R¹³)R¹⁴, —C(R¹¹)(R¹²)—CO—N(R¹³)—OR¹⁴, —C(R¹¹)(R¹²)—PO(OR¹³)₂, C₃-C₈-cycloalkyl, C₃-C₈-cycloalkyl-C₁-C₄-alkyl or phenyl which may be unsubstituted or may carry one, two, three or four substituents, in each case selected from the group consisting of cyano, nitro, amino, hydroxyl, halogen, C₁-C₄-alkyl, C₁-C₄-alkoxy, C₁-C₄-alkylsulfonyl, (C₁-C₄-alkyl)carbonyl and (C₁-C₄-alkoxy)carbonyl;

in particular hydrogen, C₁-C₄-haloalkyl, C₁-C₄-alkoxy-C₁-C₄-alkyl, C₂-C₆-alkenyl, C₂-C₆-haloalkenyl, C₂-C₆-alkynyl, —CH(R¹¹)(R¹²), —C(R¹¹)(R¹²)—CO—OR¹³, —C(R¹¹)(R¹²)—CO—N(R¹³)R¹⁴, C₃-C₈-cycloalkyl-C₁-C₄-alkyl or C₃-C₈-cycloalkyl, particularly preferably hydrogen, C₁-C₆-alkyl, C₁-C₄-alkoxy-C₁-C₄-alkyl, C₂-C₆-alkenyl, C₂-C₆-alkynyl, —C(R¹¹)(R¹²) —CO—OR¹³ or C₃-C₈-cycloalkyl;

in which the variables R¹¹, R¹², R¹³, R¹⁴ and R¹⁵ independently of one another are preferably as defined below:

R¹¹, R¹² independently of one another are hydrogen, C₁-C₄-alkyl, C₁-C₄-alkoxy-C₁-C₄-alkyl, C₁-C₄-alkylthio-C₁-C₄-alkyl, (C₁-C₄-alkoxy)carbonyl-C₁-C₄-alkyl or phenyl-C₁-C₄-alkyl, in particular hydrogen or C₁-C₄-alkyl, especially methyl;

R¹³, R¹⁴ independently of one another are hydrogen, C₁-C₆-alkyl, C₁-C₆-haloalkyl, C₁-C₄-alkoxy-C₁-C₄-alkyl, C₂-C₆-alkenyl, C₂-C₆-haloalkenyl, C₂-C₆-alkynyl, C₃-C₈-cycloalkyl, C₃-C₈-cycloalkyl-C₁-C₄-alkyl, phenyl, phenyl-C₁-C₄-alkyl, in particular hydrogen or C₁-C₄-alkyl;

R¹⁵ is C₁-C₆-alkyl; and

R¹⁰ is hydrogen, C₁-C₆-alkyl, C₁-C₄-alkoxycarbonyl-C₁-C₄-alkyl, C₂-C₆-alkenyl, in particular C₁-C₄-alkyl.

Compounds I in which Q=C—H and the variables X, R⁴, R⁵ and R⁶ are as defined above are hereinbelow referred to as compounds IA. Compounds of the formula IA are particularly preferred according to the invention. Compounds where Q=N are hereinbelow referred to as compounds IB, and they are a further preferred embodiment of the invention.

If Q in formula I is a group C—R⁷, then it is also possible for XR⁶ and R⁷ to form a 3- or 4-membered chain which, in addition to carbon, may contain 1, 2 or 3 heteroatoms selected from the group consisting of nitrogen, oxygen and sulfur atoms. With the phenyl ring in formula I, this chain forms a fused ring which may be unsubstituted or may for its part carry one, two or three substituents, and whose members may also include one or two nonadjacent carbonyl, thiocarbonyl or sulfonyl groups. Hereinbelow, such compounds are referred to as compounds IC.

Among the compounds IC, preference is given to those compounds I in which R⁷ together with X—R⁶ in formula I is a chain of the formulae O—C(R¹⁶,R¹⁷)—CO—N(R¹⁸)—, S—C(R¹⁶,R¹⁷)—CO—N(R¹⁸)— and, particularly preferably, N═C(R¹⁹)—O— or N═C(R¹⁹)—S—, where the variables R¹⁶ to R¹⁹ are as defined below:

R¹⁶, R¹⁷ independently of one another are hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-₈-cycloalkyl, phenyl or phenyl-C₁-C₄-alkyl, in particular hydrogen or C₁-C₆-alkyl;

R¹⁸ is hydrogen, hydroxyl, C₁-C₆-alkyl, C₁-C₆-haloalkyl, C₂-C₆-alkenyl, C₂-C₆-haloalkenyl, C₂-C₆-alkynyl, C₁-C₄-alkoxy, C₁-C₄-haloalkoxy, C₃-C₆-alkenyloxy, C₃-C₆-alkynyloxy, C₁-C₄-alkylsulfonyl, C₁-C₄-haloalkylsulfonyl, C₁-C₄-alkylcarbonyl, C₁-C₄-haloalkylcarbonyl, C₁-C₄-alkoxycarbonyl, C₁-C₄-alkoxy-C₁-C₄-alkyl, C₁-C₄-alkoxycarbonyl-C₁-C₄-alkyl, C₁-C₄-alkoxycarbonyl-C₁-C₄-alkoxy, di(C₁-C₄-alkyl)aminocarbonyl, di(C₁-C₄-alkyl)aminocarbonyl-C₁-C₄-alkyl, di(C₁-C₄-alkyl)aminocarbonyl-C₁-C₄-alkoxy, phenyl, phenyl-C₁-C₄-alkyl, C₃-C₈-cycloalkyl, C₃-C₈-cycloalkyl-C₁-C₄-alkyl, 3-, 4-, 5-, 6- or 7-membered, preferably 5- or 6-membered, preferably saturated heterocyclyl which contains one or two, preferably one, ring heteroatom selected from the group consisting of oxygen, nitrogen and sulfur,

R¹⁹ is hydrogen, halogen, cyano, amino, C₁-C₆-alkyl, C₁-C₆-haloalkyl, C₂-C₆-alkenyl, C₂-C₆-haloalkenyl, C₂-C₆-alkynyl, C₁-C₄-alkoxy, C₁-C₄-haloalkoxy, C₃-C₆-alkenyloxy, C₃-C₆-alkynyloxy, C₁-C₄-alkylamino, di(C₁-C₄-alkyl)amino, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₁-C₄-alkylcarbonyl, C₁-C₄-haloalkylcarbonyl, C₁-C₄-alkoxy-C₁-C₄-alkyl, C₁-C₄-alkoxycarbonyl, C₁-C₄-alkoxycarbonyl-C₁-C₄-alkyl, C₁-C₄-alkoxycarbonyl-C₁-C₄-alkoxy, C₁-C₄-alkoxycarbonyl-C₁-C₄-alkylthio, di(C₁-C₄-alkyl)aminocarbonyl, di(C₁-C₄-alkyl)aminocarbonyl-C₁-C₄-alkyl, di(C₁-C₄-alkyl)aminocarbonyl-C₁-C₄-alkoxy, di(C₁-C₄-alkyl)aminocarbonyl-C₁-C₄-alkylthio, C₃-C₈-cycloalkyl, phenyl, phenyl-C₁-C₄-alkyl, C₃-C₈-cycloalkyl-C₁-C₄-alkyl, 3-, 4-, 5-, 6- or 7-membered, preferably 5- or 6-membered, preferably saturated heterocyclyl which contains one or two, preferably one, ring heteroatom selected from the group consisting of oxygen, nitrogen and sulfur.

The variables R¹⁸ and R¹⁹ are preferably as defined below:

R¹⁸ is hydrogen, hydroxyl, C₁-C₆-alkyl, C₁-C₆-haloalkyl, C₂-C₆-alkenyl, C₂-C₆-alkynyl, C₁-C₄-alkoxy, C₁-C₄-haloalkoxy, C₃-C₆-alkenyloxy, C₃-C₆-alkynyloxy, C₁-C₄-alkoxy-C₁-C₄-alkyl, C₁-C₄-alkoxycarbonyl-C₁-C₄-alkyl, C₁-C₄-alkoxycarbonyl-C₁-C₄-alkoxy, C₃-C₈-cycloalkyl, C₃-C₈-cycloalkyl-C₁-C₄-alkyl or phenyl-C₁-C₄-alkyl or 3-, 4-, 5- or 6-membered, preferably 5- or 6-membered, preferably saturated heterocyclyl which contains one ring heteroatom selected from the group consisting of oxygen, nitrogen and sulfur;

R¹⁹ is hydrogen, halogen, amino, C₁-C₆-alkyl, C₁-C₆-haloalkyl, C₂-C₆-alkenyl, C₂-C₆-haloalkenyl, C₂-C₆-alkynyl, C₁-C₄-alkoxy, C₁-C₄-haloalkoxy, C₃-C₆-alkenyloxy, C₃-C₆-alkynyloxy, C₁-C₄-alkylamino, di(C₁-C₄-alkyl)amino, C₁-C₄-alkylthio, C₁-C₄-alkoxy-C₁-C₄-alkyl, C₁-C₄-alkoxycarbonyl-C₁-C₄-alkyl, C₁-C₄-alkoxycarbonyl-C₁-C₄-alkoxy, C₁-C₄-alkoxycarbonyl-C₁-C₄-alkylthio, C₃-C₈-cycloalkyl, phenyl, phenyl-C₁-C₄-alkyl, C₃-C₈-cycloalkyl-C₁-C₄-alkyl, 3-, 4-, 5- or 6-membered, preferably 5- or 6-membered, preferably saturated heterocyclyl which contains one ring heteroatom selected from the group consisting of oxygen, nitrogen and sulfur.

In the compounds IC, R⁴ and R⁵ independently of one another have the meanings given above as being preferred, in particular in combination.

Particular preference is given to compounds of the formula IA where R³═CF₃ and R¹═Cl in which R² and R^{2′} independently of one another are selected from the group consisting of hydrogen and methyl and in which the variables X, R⁴, R⁵ and R⁶ are as defined above and, in particular together, have the meanings given in each case in one row of Table 1.

Examples of such compounds are the compounds of the formula IAa given below in which R⁴, R⁵ and X—R⁶ together have in each case the meanings given in one row of Table 1 (compounds IAa.1-IAa.798).

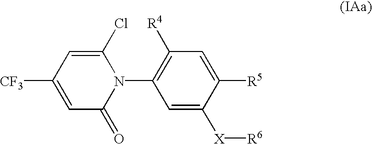

Examples of such compounds are also the compounds of the formula IAb given below in which R⁴, R⁵ and X—R⁶ together have in each case the meanings given in one row of Table 1 (compounds IAb.1-IAb.798).

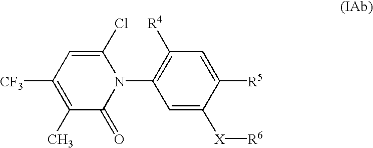

Examples of such compounds are also the compounds of the formula IAc given below in which R⁴, R⁵ and X—R⁶ together have in each case the meanings given in one row of Table 1 (compounds IAc.1-IAc.798).

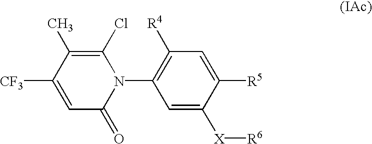

Examples of such compounds are also the compounds of the formula IAd given below in which R⁴, R⁵ and X—R⁶ together have in each case the meanings given in one row of Table 1 (compounds IAd.1-IAd.798).

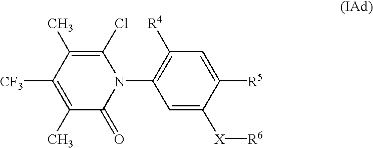

Examples of such compounds are also the compounds of formulae IAe, IAf, IAg and IAh given below in which R⁴, R⁵ and X—R⁶ together have in each case the meanings given in one row of Table 1 (compounds IAe.1-IAe.798, IAf.1-IAf.798, IAg.1-IAg.798 and IAh.1-IAh.798).

TABLE 1

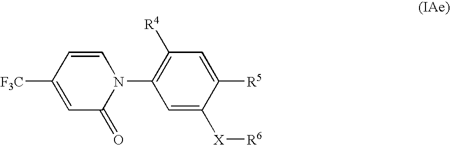

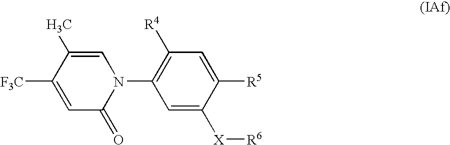

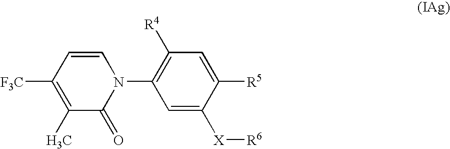

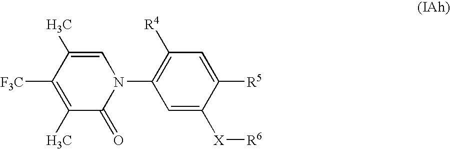

| No. | R⁴ | R⁵ | X—R⁶ | |
|---|---|---|---|---|
| 1 | F | Cl | H | |
| 2 | F | Cl | F | |
| 3 | F | Cl | CH₃ | |
| 4 | F | Cl | NO₂ | |
| 5 | F | Cl | NH₂ | |
| 6 | F | Cl | OH | |
| 7 | F | Cl | OCH₃ | |
| 8 | F | Cl | OCH(CH₃)₂ | |
| 9 | F | Cl | O—CH₂CH═CH₂ | |
| 10 | F | Cl | O—CH₂C≡CH | |
| 11 | F | Cl | O—CH(CH₃)C≡CH | |
| 12 | F | Cl | O-cyclopentyl | |
| 13 | F | Cl | OCH₂COOH | |
| 14 | F | Cl | OCH₂COO—CH₃ | |
| 15 | F | Cl | OCH₂COO—CH₂CH₃ | |
| 16 | F | Cl | OCH₂COO—CH₂CH═CH₂ | |
| 17 | F | Cl | OCH₂COO—CH₂C≡CH | |
| 18 | F | Cl | OCH₂COO—CH₂CH₂OCH₃ | |
| 19 | F | Cl | OCH₂CONH—CH₃ | |
| 20 | F | Cl | OCH₂CON(CH₃)₂ | |
| 21 | F | Cl | OCH(CH₃)COOH | |
| 22 | F | Cl | OCH(CH₃)COO—CH₃ | |
| 23 | F | Cl | OCH(CH₃)COO—CH₂CH₃ | |
| 24 | F | Cl | OCH(CH₃)COO—CH₂CH═CH₂ | |
| 25 | F | Cl | OCH(CH₃)COO—CH₂C≡CH | |
| 26 | F | Cl | OCH(CH₃)COO—CH₂CH₂OCH₃ | |
| 27 | F | Cl | OCH(CH₃)CONH—CH₃ | |
| 28 | F | Cl | OCH(CH₃)CON(CH₃)₂ | |
| 29 | F | Cl | OC(CH₃)₂COO—CH₃ | |
| 30 | F | Cl | OC(CH₃)₂COO—CH₂CHCH₂ | |
| 31 | F | Cl | SH | |
| 32 | F | Cl | SCH₃ | |
| 33 | F | Cl | SCH(CH₃)₂ | |
| 34 | F | Cl | S—CH₂CH═CH₂ | |
| 35 | F | Cl | S—CH₂C≡CH | |
| 36 | F | Cl | S—CH(CH₃)C≡CH | |
| 37 | F | Cl | S-cyclopentyl | |
| 38 | F | Cl | SCH₂COOH | |
| 39 | F | Cl | SCH₂COO—CH₃ | |
| 40 | F | Cl | SCH₂COO—CH₂CH₃ | |
| 41 | F | Cl | SCH₂COO—CH₂CH═CH₂ | |
| 42 | F | Cl | SCH₂COO—CH₂C≡CH | |
| 43 | F | Cl | SCH₂COO—CH₂CH₂OCH₃ | |
| 44 | F | Cl | SCH₂CONH—CH₃ | |
| 45 | F | Cl | SCH₂CON(CH₃)₂ | |
| 46 | F | Cl | SCH(CH₃)COOH | |
| 47 | F | Cl | SCH(CH₃)COO—CH₃ | |
| 48 | F | Cl | SCH(CH₃)COO—CH₂CH₃ | |
| 49 | F | Cl | SCH(CH₃)COO—CH₂CH═CH₂ | |
| 50 | F | Cl | SCH(CH₃)COO—CH₂C≡CH | |
| 51 | F | Cl | SCH(CH₃)COO—CH₂CH₂OCH₃ | |
| 52 | F | Cl | SCH(CH₃)CONH—CH₃ | |
| 53 | F | Cl | SCH(CH₃)CON(CH₃)₂ | |
| 54 | F | Cl | SC(CH₃)₂COO—CH₃ | |
| 55 | F | Cl | SC(CH₃)₂COO—CH₂CH═CH₂ | |
| 56 | F | Cl | COOH | |
| 57 | F | Cl | COOCH₃ | |
| 58 | F | Cl | COOCH₂CH₃ | |
| 59 | F | Cl | COOCH(CH₃)₂ | |
| 60 | F | Cl | COO—CH₂CH═CH₂ | |
| 61 | F | Cl | COO—CH₂C≡CH | |
| 62 | F | Cl | COO-cyclopentyl | |
| 63 | F | Cl | COO—CH₂COO—CH₃ | |
| 64 | F | Cl | COO—CH₂COO—CH₂CH₃ | |
| 65 | F | Cl | COO—CH₂COO—CH₂CH═CH₂ | |
| 66 | F | Cl | COO—CH₂COO—CH₂C≡CH | |
| 67 | F | Cl | COO—CH₂COO—CH₂CH₂OCH₃ | |
| 68 | F | Cl | COO—CH(CH₃)COO—CH₃ | |
| 69 | F | Cl | COO—CH(CH₃)COO—CH₂CH₃ | |
| 70 | F | Cl | COO—CH(CH₃)COO—CH₂CH═CH₂ | |
| 71 | F | Cl | COO—CH(CH₃)COO—CH₂C≡CH | |
| 72 | F | Cl | COO—CH(CH₃)COO—CH₂CH₂OCH₃ | |
| 73 | F | Cl | COO—C(CH₃)₂COO—CH₃ | |
| 74 | F | Cl | COO—C(CH₃)₂COO—CH₂CH₃ | |
| 75 | F | Cl | COO—C(CH₃)₂COO—CH₂CH═CH₂ | |
| 76 | F | Cl | COO—C(CH₃)₂COO—CH₂C≡CH | |
| 77 | F | Cl | COO—C(CH₃)₂COO—CH₂CH₂OCH₃ | |
| 78 | F | Cl | CONH₂ | |
| 79 | F | Cl | CONHCH₃ | |
| 80 | F | Cl | CON(CH₃)₂ | |
| 81 | F | Cl | CONH—CH₂COO—CH₃ | |
| 82 | F | Cl | CONH—CH₂COO—CH₂CH═CH₂ | |
| 83 | F | Cl | CONH—CH₂COO—CH₂CH₂OCH₃ | |
| 84 | F | Cl | CONH—CH(CH₃)COO—CH₃ | |
| 85 | F | Cl | CONH—CH(CH₃)COO—CH₂CH═CH₂ | |
| 86 | F | Cl | CONH—CH(CH₃)COO—CH₂CH₂OCH₃ | |
| 87 | F | Cl | CON(CH₃)—CH₂COO—CH₃ | |
| 88 | F | Cl | CON(CH₃)—CH₂COO—CH₂CH═CH₂ | |
| 89 | F | Cl | CON(CH₃)—CH₂COO—CH₂CH₂OCH₃ | |
| 90 | F | Cl | C(═N—OCH₃)O—CH₃ | |
| 91 | F | Cl | C(═N—OCH₃)O—CH₂—COOCH₃ | |
| 92 | F | Cl | C(═N—OCH₃)O—CH₂—COO-phenyl | |
| 93 | F | Cl | C(═N—OCH₃)O—CH(CH₃)—COOCH₃ | |
| 94 | F | Cl | CH═C(Cl)COO—CH₃ | |
| 95 | F | Cl | CH═C(Cl)COO—CH₂CH₃ | |
| 96 | F | Cl | CH═C(Cl)COO—CH₂CH═CH₂ | |
| 97 | F | Cl | CH═C(Cl)COO—CH₂COOCH₃ | |
| 98 | F | Cl | CH═C(Cl)COO—CH(CH₃)COOCH₃ | |
| 99 | F | Cl | CH═C(Cl)CON(CH₃)₂ | |
| 100 | F | Cl | CH═C(Cl)CON(CH₃)—CH₂COOCH₃ | |
| 101 | F | Cl | CH═C(Cl)CONH—CH(CH₃)COOCH₃ | |
| 102 | F | Cl | CH═C(Br)COO—CH₃ | |
| 103 | F | Cl | CH═C(Br)COO—CH₂CH₃ | |
| 104 | F | Cl | CH═C(CH₃)COO—CH₃ | |
| 105 | F | Cl | CH═C(CH₃)COO—CH₂CH₃ | |
| 106 | F | Cl | CH₂—CH(Cl)—COO—CH₃ | |
| 107 | F | Cl | CH₂—CH(Cl)—COO—CH₂CH₃ | |
| 108 | F | Cl | CHO | |
| 109 | F | Cl | CH═N—OCH₃ | |
| 110 | F | Cl | CH═N—OCH₂CH₃ | |
| 111 | F | Cl | CH═N—OCH(CH₃)COOCH₃ | |
| 112 | F | Cl | SO₂Cl | |
| 113 | F | Cl | SO₂NH₂ | |
| 114 | F | Cl | SO₂NHCH₃ | |
| 115 | F | Cl | SO₂N(CH₃)₂ | |
| 116 | F | Cl | NH—CH₂C≡CH | |
| 117 | F | Cl | NHCH(CH₃)COOCH₃ | |
| 118 | F | Cl | N(CH₃)—CH₂C≡CH | |
| 119 | F | Cl | NH(SO₂CH₃) | |
| 120 | F | Cl | N(CH₃)(SO₂CH₃) | |
| 121 | F | Cl | N(SO₂CH₃)₂ | |
| 122 | F | CN | H | |
| 123 | F | CN | F | |
| 124 | F | CN | CH₃ | |
| 125 | F | CN | NO₂ | |
| 126 | F | CN | NH₂ | |
| 127 | F | CN | OH | |
| 128 | F | CN | OCH₃ | |
| 129 | F | CN | OCH(CH₃)₂ | |
| 130 | F | CN | O—CH₂CH═CH₂ | |
| 131 | F | CN | O—CH₂C≡CH | |
| 132 | F | CN | O—CH(CH₃)C≡CH | |
| 133 | F | CN | O-cyclopentyl | |
| 134 | F | CN | OCH₂COOH | |
| 135 | F | CN | OCH₂COO—CH₃ | |
| 136 | F | CN | OCH₂COO—CH₂CH₃ | |
| 137 | F | CN | OCH₂COO—CH₂CH═CH₂ | |
| 138 | F | CN | OCH₂COO—CH₂C≡CH | |
| 139 | F | CN | OCH₂COO—CH₂CH₂OCH₃ | |
| 140 | F | CN | OCH₂CONH—CH₃ | |
| 141 | F | CN | OCH₂CON(CH₃)₂ | |
| 142 | F | CN | OCH(CH₃)COOH | |
| 143 | F | CN | OCH(CH₃)COO—CH₃ | |
| 144 | F | CN | OCH(CH₃)COO—CH₂CH₃ | |
| 145 | F | CN | OCH(CH₃)COO—CH₂CH═CH₂ | |
| 146 | F | CN | OCH(CH₃)COO—CH₂C≡CH | |
| 147 | F | CN | OCH(CH₃)COO—CH₂CH₂OCH₃ | |
| 148 | F | CN | OCH(CH₃)CONH—CH₃ | |
| 149 | F | CN | OCH(CH₃)CON(CH₃)₂ | |
| 150 | F | CN | OC(CH₃)₂COO—CH₃ | |
| 151 | F | CN | OC(CH₃)₂COO—CH₂CH═CH₂ | |
| 152 | F | CN | SH | |
| 153 | F | CN | SCH₃ | |
| 154 | F | CN | SCH(CH₃)₂ | |
| 155 | F | CN | S—CH₂CH═CH₂ | |
| 156 | F | CN | S—CH₂C≡CH | |
| 157 | F | CN | S—CH(CH₃)C≡CH | |
| 158 | F | CN | S-cyclopentyl | |
| 159 | F | CN | SCH₂COOH | |
| 160 | F | CN | SCH₂COO—CH₃ | |
| 161 | F | CN | SCH₂COO—CH₂CH₃ | |
| 162 | F | CN | SCH₂COO—CH₂CH═CH₂ | |
| 163 | F | CN | SCH₂COO—CH₂C≡CH | |
| 164 | F | CN | SCH₂COO—CH₂CH₂OCH₃ | |
| 165 | F | CN | SCH₂CONH—CH₃ | |
| 166 | F | CN | SCH₂CON(CH₃)₂ | |
| 167 | F | CN | SCH(CH₃)COOH | |
| 168 | F | CN | SCH(CH₃)COO—CH₃ | |
| 169 | F | CN | SCH(CH₃)COO—CH₂CH₃ | |
| 170 | F | CN | SCH(CH₃)COO—CH₂CH═CH₂ | |
| 171 | F | CN | SCH(CH₃)COO—CH₂C≡CH | |
| 172 | F | CN | SCH(CH₃)COO—CH₂CH₂OCH₃ | |
| 173 | F | CN | SCH(CH₃)CONH—CH₃ | |
| 174 | F | CN | SCH(CH₃)CON(CH₃)₂ | |
| 175 | F | CN | SC(CH₃)₂COO—CH₃ | |
| 176 | F | CN | SC(CH₃)₂COO—CH₂CH═CH₂ | |
| 177 | F | CN | COOH | |
| 178 | F | CN | COOCH₃ | |
| 179 | F | CN | COOCH₂CH₃ | |
| 180 | F | CN | COOCH(CH₃)₂ | |
| 181 | F | CN | COO—CH₂CH═CH₂ | |
| 182 | F | CN | COO—CH₂C≡CH | |
| 183 | F | CN | COO-cyclopentyl | |
| 184 | F | CN | COO—CH₂COO—CH₃ | |
| 185 | F | CN | COO—CH₂COO—CH₂CH₃ | |
| 186 | F | CN | COO—CH₂COO—CH₂CH═CH₂ | |
| 187 | F | CN | COO—CH₂COO—CH₂C≡CH | |
| 188 | F | CN | COO—CH₂COO—CH₂CH₂OCH₃ | |
| 189 | F | CN | COO—CH(CH₃)COO—CH₃ | |
| 190 | F | CN | COO—CH(CH₃)COO—CH₂CH₃ | |
| 191 | F | CN | COO—CH(CH₃)COO—CH₂CH═CH₂ | |
| 192 | F | CN | COO—CH(CH₃)COO—CH₂C≡CH | |
| 193 | F | CN | COO—CH(CH₃)COO—CH₂CH₂OCH₃ | |
| 194 | F | CN | COO—C(CH₃)₂COO—CH₃ | |
| 195 | F | CN | COO—C(CH₃)₂COO—CH₂CH₃ | |
| 196 | F | CN | COO—C(CH₃)₂COO—CH₂CH═CH₂ | |
| 197 | F | CN | COO—C(CH₃)₂COO—CH₂C≡CH | |
| 198 | F | CN | COO—C(CH₃)₂COO—CH₂CH₂OCH₃ | |
| 199 | F | CN | CONH₂ | |
| 200 | F | CN | CONHCH₃ | |
| 202 | F | CN | CONH—CH₂COO—CH₃ | |
| 203 | F | CN | CONH—CH₂COO—CH₂CH═CH₂ | |
| 204 | F | CN | CONH—CH₂COO—CH₂CH₂OCH₃ | |
| 205 | F | CN | CONH—CH(CH₃)COO—CH₃ | |
| 206 | F | CN | CONH—CH(CH₃)COO—CH₂CH═CH₂ | |
| 207 | F | CN | CONH—CH(CH₃)COO—CH₂CH₂OCH₃ | |
| 208 | F | CN | CON(CH₃)—CH₂COO—CH₃ | |
| 209 | F | CN | CON(CH₃)—CH₂COO—CH₂CH═CH₂ | |
| 210 | F | CN | CON(CH₃)—CH₂COO—CH₂CH₂OCH₃ | |
| 211 | F | CN | C(═N—OCH₃)O—CH₃ | |
| 212 | F | CN | C(═N—OCH₃)O—CH₂—COOCH₃ | |
| 213 | F | CN | C(═N—OCH₃)O—CH₂—COO-phenyl | |
| 214 | F | CN | C(═N—OCH₃)O—CH(CH₃)—COOCH₃ | |
| 215 | F | CN | CH═C(Cl)COO—CH₃ | |
| 216 | F | CN | CH═C(Cl)COO—CH₂CH₃ | |
| 217 | F | CN | CH═C(Cl)COO—CH₂CH═CH₂ | |
| 218 | F | CN | CH═C(Cl)COO—CH₂COOCH₃ | |
| 219 | F | CN | CH═C(Cl)COO—CH(CH₃)COOCH₃ | |
| 220 | F | CN | CH═C(Cl)CON(CH₃)₂ | |
| 221 | F | CN | CH═C(Cl)CON(CH₃)—CH₂COOCH₃ | |
| 222 | F | CN | CH═C(Cl)CONH—CH(CH₃)COOCH₃ | |
| 223 | F | CN | CH═C(Br)COO—CH₃ | |
| 224 | F | CN | CH═C(Br)COO—CH₂CH₃ | |
| 225 | F | CN | CH═C(CH₃)COO—CH₃ | |
| 226 | F | CN | CH═C(CH₃)COO—CH₂CH₃ | |
| 227 | F | CN | CH₂—CH(Cl)—COO—CH₃ | |
| 228 | F | CN | CH₂—CH(Cl)—COO—CH₂CH₃ | |
| 229 | F | CN | CHO | |
| 230 | F | CN | CH═N—OCH₃ | |
| 231 | F | CN | CH═N—OCH₂CH₃ | |
| 232 | F | CN | CH═N—OCH(CH₃)COOCH₃ | |
| 233 | F | CN | SO₂Cl | |
| 234 | F | CN | SO₂NH₂ | |
| 235 | F | CN | SO₂NHCH₃ | |
| 236 | F | CN | SO₂N(CH₃)₂ | |
| 237 | F | CN | NH—CH₂C≡CH | |
| 238 | F | CN | NHCH(CH₃)COOCH₃ | |
| 239 | F | CN | N(CH₃)—CH₂C≡CH | |
| 240 | F | CN | NH(SO₂CH₃) | |
| 241 | F | CN | N(CH₃)(SO₂CH₃) | |
| 242 | F | CN | N(SO₂CH₃)₂ | |
| 243 | Cl | Cl | H | |
| 244 | Cl | Cl | F | |
| 245 | Cl | Cl | CH₃ | |
| 246 | Cl | Cl | NO₂ | |
| 247 | Cl | Cl | NH₂ | |
| 248 | Cl | Cl | OH | |
| 249 | Cl | Cl | OCH₃ | |
| 250 | Cl | Cl | OCH(CH₃)₂ | |
| 251 | Cl | Cl | O—CH₂CH═CH₂ | |
| 252 | Cl | Cl | O—CH₂C≡CH | |
| 253 | Cl | Cl | O—CH(CH₃)C≡CH | |
| 254 | Cl | Cl | O-cyclopentyl | |
| 255 | Cl | Cl | OCH₂COOH | |
| 256 | Cl | Cl | OCH₂COO—CH₃ | |
| 257 | Cl | Cl | OCH₂COO—CH₂CH₃ | |
| 258 | Cl | Cl | OCH₂COO—CH₂CH═CH₂ | |
| 259 | Cl | Cl | OCH₂COO—CH₂C≡CH | |
| 260 | Cl | Cl | OCH₂COO—CH₂CH₂OCH₃ | |
| 261 | Cl | Cl | OCH₂CONH—CH₃ | |
| 262 | Cl | Cl | OCH₂CON(CH₃)₂ | |
| 263 | Cl | Cl | OCH(CH₃)COOH | |
| 264 | Cl | Cl | OCH(CH₃)COO—CH₃ | |
| 265 | Cl | Cl | OCH(CH₃)COO—CH₂CH₃ | |
| 266 | Cl | Cl | OCH(CH₃)COO—CH₂CH═CH₂ | |
| 267 | Cl | Cl | OCH(CH₃)COO—CH₂C≡CH | |
| 268 | Cl | Cl | OCH(CH₃)COO—CH₂CH₂OCH₃ | |
| 269 | Cl | Cl | OCH(CH₃)CONH—CH₃ | |
| 270 | Cl | Cl | OCH(CH₃)CON(CH₃)₂ | |
| 271 | Cl | Cl | OC(CH₃)₂COO—CH₃ | |
| 272 | Cl | Cl | OC(CH₃)₂COO—CH₂CH═CH₂ | |
| 273 | Cl | Cl | SH | |
| 274 | Cl | Cl | SCH₃ | |
| 275 | Cl | Cl | SCH(CH₃)₂ | |
| 276 | Cl | Cl | S—CH₂CH═CH₂ | |
| 277 | Cl | Cl | S—CH₂C≡CH | |
| 278 | Cl | Cl | S—CH(CH₃)C≡CH | |
| 279 | Cl | Cl | S-cyclopentyl | |
| 280 | Cl | Cl | SCH₂COOH | |
| 281 | Cl | Cl | SCH₂COO—CH₃ | |
| 282 | Cl | Cl | SCH₂COO—CH₂CH₃ | |
| 283 | Cl | Cl | SCH₂COO—CH₂CH═CH₂ | |
| 284 | Cl | Cl | SCH₂COO—CH₂C≡CH | |
| 285 | Cl | Cl | SCH₂COO—CH₂CH₂OCH₃ | |
| 286 | Cl | Cl | SCH₂CONH—CH₃ | |
| 287 | Cl | Cl | SCH₂COH(CH₃)₂ | |
| 288 | Cl | Cl | SCH(CH₃)COOH | |
| 289 | Cl | Cl | SCH(CH₃)COO—CH₃ | |
| 290 | Cl | Cl | SCH(CH₃)COO—CH₂CH₃ | |
| 291 | Cl | Cl | SCH(CH₃)COO—CH₂CH═CH₂ | |
| 292 | Cl | Cl | SCH(CH₃)COO—CH₂C≡CH | |
| 293 | Cl | Cl | SCH(CH₃)COO—CH₂CH₂OCH₃ | |
| 294 | Cl | Cl | SCH(CH₃)CONH—CH₃ | |
| 295 | Cl | Cl | SCH(CH₃)CON(CH₃)₂ | |
| 296 | Cl | Cl | SC(CH₃)₂COO—CH₃ | |
| 297 | Cl | Cl | SC(CH₃)₂COO—CH₂CH═CH₂ | |
| 298 | Cl | Cl | COOH | |
| 299 | Cl | Cl | COOCH₃ | |
| 300 | Cl | Cl | COOCH₂CH₃ | |
| 301 | Cl | Cl | COOCH(CH₃)₂ | |
| 302 | Cl | Cl | COO—CH₂CH═CH₂ | |
| 303 | Cl | Cl | COO—CH₂C≡CH | |
| 304 | Cl | Cl | COO-cyclopentyl | |
| 305 | Cl | Cl | COO—CH₂COO—CH₃ | |
| 306 | Cl | Cl | COO—CH₂COO—CH₂CH₃ | |
| 307 | Cl | Cl | COO—CH₂COO—CH₂CH═CH₂ | |
| 308 | Cl | Cl | COO—CH₂COO—CH₂C≡CH | |
| 309 | Cl | Cl | COO—CH₂COO—CH₂CH₂OCH₃ | |
| 310 | Cl | Cl | COO—CH(CH₃)COO—CH₃ | |
| 311 | Cl | Cl | COO—CH(CH₃)COO—CH₂CH₃ | |
| 312 | Cl | Cl | COO—CH(CH₃)COO—CH₂CH═CH₂ | |
| 313 | Cl | Cl | COO—CH(CH₃)COO—CH₂C≡CH | |
| 314 | Cl | Cl | COO—CH(CH₃)COO—CH₂CH₂OCH₃ | |
| 315 | Cl | Cl | COO—C(CH₃)₂COO—CH₃ | |
| 316 | Cl | Cl | COO—C(CH₃)₂COO—CH₂CH₃ | |
| 317 | Cl | Cl | COO—C(CH₃)₂COO—CH₂CH═CH₂ | |
| 318 | Cl | Cl | COO—C(CH₃)₂COO—CH₂C≡CH | |
| 319 | Cl | Cl | COO—C(CH₃)₂COO—CH₂CH₂OCH₃ | |
| 320 | Cl | Cl | CONH₂ | |
| 321 | Cl | Cl | CONHCH₃ | |
| 322 | Cl | Cl | CON(CH₃)₂ | |
| 323 | Cl | Cl | CONH—CH₂COO—CH₃ | |
| 324 | Cl | Cl | CONH—CH₂COO—CH₂CH═CH₂ | |
| 325 | Cl | Cl | CONH—CH₂COO—CH₂CH₂OCH₃ | |
| 326 | Cl | Cl | CONH—CH(CH₃)COO—CH₃ | |
| 327 | Cl | Cl | CONH—CH(CH₃)COO—CH₂CH═CH₂ | |
| 328 | Cl | Cl | CONH—CH(CH₃)COO—CH₂CH₂OCH₃ | |
| 329 | Cl | Cl | CON(CH₃)—CH₂COO—CH₃ | |
| 330 | Cl | Cl | CON(CH₃)—CH₂COO—CH₂CH═CH₂ | |
| 331 | Cl | Cl | CON(CH₃)—CH₂COO—CH₂CH₂OCH₃ | |
| 332 | Cl | Cl | C(═N—OCH₃)O—CH₃ | |
| 333 | Cl | Cl | C(═N—OCH₃)O—CH₂—COOCH₃ | |
| 334 | Cl | Cl | C(═N—OCH₃)O—CH₂—COO-phenyl | |
| 335 | Cl | Cl | C(═N—OCH₃)O—CH(CH₃)—COOCH₃ | |
| 336 | Cl | Cl | CH═C(Cl)COO—CH₃ | |
| 337 | Cl | Cl | CH═C(Cl)COO—CH₂CH₃ | |
| 338 | Cl | Cl | CH═C(Cl)COO—CH₂CH═CH₂ | |
| 339 | Cl | Cl | CH═C(Cl)COO—CH₂COOCH₃ | |
| 340 | Cl | Cl | CH═C(Cl)COO—CH(CH₃)COOCH₃ | |
| 341 | Cl | Cl | CH═C(Cl)CON(CH₃)₂ | |
| 342 | Cl | Cl | CH═C(Cl)CON(CH₃)—CH₂COOCH₃ | |
| 343 | Cl | Cl | CH═C(Cl)CONH—CH(CH₃)COOCH₃ | |
| 344 | Cl | Cl | CH═C(Br)COO—CH₃ | |
| 345 | Cl | Cl | CH═C(Br)COO—CH₂CH₃ | |
| 346 | Cl | Cl | CH═C(CH₃)COO—CH₃ | |
| 347 | Cl | Cl | CH═C(CH₃)COO—CH₂CH₃ | |
| 348 | Cl | Cl | CH₂—CH(Cl)—COO—CH₃ | |
| 349 | Cl | Cl | CH₂—CH(Cl)—COO—CH₂CH₃ | |
| 350 | Cl | Cl | CHO | |
| 351 | Cl | Cl | CH═N—OCH₃ | |
| 352 | Cl | Cl | CH═N—OCH₂CH₃ | |
| 353 | Cl | Cl | CH═N—OCH(CH₃)COOCH₃ | |
| 354 | Cl | Cl | SO₂Cl | |
| 355 | Cl | Cl | SO₂NH₂ | |
| 356 | Cl | Cl | SO₂NHCH₃ | |
| 357 | Cl | Cl | SO₂N(CH₃)₂ | |
| 358 | Cl | Cl | NH—CH₂C≡CH | |
| 359 | Cl | Cl | NHCH(CH₃)COOCH₃ | |
| 360 | Cl | Cl | N(CH₃)—CH₂C≡CH | |
| 361 | Cl | Cl | NH(SO₂CH₃) | |
| 362 | Cl | Cl | N(CH₃)(SO₂CH₃) | |
| 363 | Cl | Cl | N(SO₂CH₃)₂ | |
| 364 | Cl | CN | H | |
| 365 | Cl | CN | F | |
| 366 | Cl | CN | CH₃ | |
| 367 | Cl | CN | NO₂ | |
| 368 | Cl | CN | NH₂ | |
| 369 | Cl | CN | OH | |
| 370 | Cl | CN | OCH₃ | |
| 371 | Cl | CN | OCH(CH₃)₂ | |
| 372 | Cl | CN | O—CH₂CH═CH₂ | |
| 373 | Cl | CN | O—CH₂C≡CH | |
| 374 | Cl | CN | O—CH(CH₃)C≡CH | |
| 375 | Cl | CN | O-cyclopentyl | |
| 376 | Cl | CN | OCH₂COOH | |
| 377 | Cl | CN | OCH₂COO—CH₃ | |
| 378 | Cl | CN | OCH₂COO—CH₂CH₃ | |
| 379 | Cl | CN | OCH₂COO—CH₂CH═CH₂ | |
| 380 | Cl | CN | OCH₂COO—CH₂C≡CH | |
| 381 | Cl | CN | OCH₂COO—CH₂CH₂OCH₃ | |
| 382 | Cl | CN | OCH₂CONH—CH₃ | |
| 383 | Cl | CN | OCH₂CON(CH₃)₂ | |
| 384 | Cl | CN | OCH(CH₃)COOH | |
| 385 | Cl | CN | OCH(CH₃)COO—CH₃ | |
| 386 | Cl | CN | OCH(CH₃)COO—CH₂CH₃ | |
| 387 | Cl | CN | OCH(CH₃)COO—CH₂CH═CH₂ | |
| 388 | Cl | CN | OCH(CH₃)COO—CH₂C≡CH | |
| 389 | Cl | CN | OCH(CH₃)COO—CH₂CH₂OCH₃ | |
| 390 | Cl | CN | OCH(CH₃)CONH—CH₃ | |
| 391 | Cl | CN | OCH(CH₃)CON(CH₃)₂ | |
| 392 | Cl | CN | OC(CH₃)₂COO—CH₃ | |
| 393 | Cl | CN | OC(CH₃)₂COO—CH₂CH═CH₂ | |
| 394 | Cl | CN | SH | |
| 395 | Cl | CN | SCH₃ | |
| 396 | Cl | CN | SCH(CH₃)₂ | |
| 397 | Cl | CN | S—CH₂CH═CH₂ | |
| 398 | Cl | CN | S—CH₂C≡CH | |
| 399 | Cl | CN | S—CH(CH₃)C≡CH | |
| 400 | Cl | CN | S-cyclopentyl | |
| 401 | Cl | CN | SCH₂COOH | |
| 402 | Cl | CN | SCH₂COO—CH₃ | |
| 403 | Cl | CN | SCH₂COO—CH₂CH₃ | |
| 404 | Cl | CN | SCH₂COO—CH₂CH═CH₂ | |
| 405 | Cl | CN | SCH₂COO—CH₂C≡CH | |
| 406 | Cl | CN | SCH₂COO—CH₂CH₂OCH₃ | |
| 407 | Cl | CN | SCH₂CONH—CH₃ | |
| 408 | Cl | CN | SCH₂CON(CH₃)₂ | |
| 409 | Cl | CN | SCH(CH₃)COOH | |
| 410 | Cl | CN | SCH(CH₃)COO—CH₃ | |
| 411 | Cl | CN | SCH(CH₃)COO—CH₂CH₃ | |
| 412 | Cl | CN | SCH(CH₃)COO—CH₂CH═CH₂ | |
| 413 | Cl | CN | SCH(CH₃)COO—CH₂C≡CH | |
| 414 | Cl | CN | SCH(CH₃)COO—CH₂CH₂OCH₃ | |
| 415 | Cl | CN | SCH(CH₃)CONH—CH₃ | |
| 416 | Cl | CN | SCH(CH₃)CON(CH₃)₂ | |
| 417 | Cl | CN | SC(CH₃)₂COO—CH₃ | |
| 418 | Cl | CN | SC(CH₃)₂COO—CH₂CH═CH₂ | |
| 419 | Cl | CN | COOH | |
| 420 | Cl | CN | COOCH₃ | |
| 421 | Cl | CN | COOCH₂CH₃ | |
| 422 | Cl | CN | COOCH(CH₃)₂ | |
| 423 | Cl | CN | COO—CH₂CH═CH₂ | |
| 424 | Cl | CN | COO—CH₂C≡CH | |
| 425 | Cl | CN | COO-cyolopentyl | |
| 426 | Cl | CN | COO—CH₂COO—CH₃ | |
| 427 | Cl | CN | COO—CH₂COO—CH₂CH₃ | |
| 428 | Cl | CN | COO—CH₂COO—CH₂CH═CH₂ | |
| 429 | Cl | CN | COO—CH₂COO—CH₂C≡CH | |
| 430 | Cl | CN | COO—CH₂COO—CH₂CH₂OCH₃ | |
| 431 | Cl | CN | COO—CH(CH₃)COO—CH₃ | |
| 432 | Cl | CN | COO—CH(CH₃)COO—CH₂CH₃ | |
| 433 | Cl | CN | COO—CH(CH₃)COO—CH₂CH═CH₂ | |
| 434 | Cl | CN | COO—CH(CH₃)COO—CH₂C≡CH | |
| 435 | Cl | CN | COO—CH(CH₃)COO—CH₂CH₂OCH₃ | |
| 436 | Cl | CN | COO—C(CH₃)₂COO—CH₃ | |
| 437 | Cl | CN | COO—C(CH₃)₂COO—CH₂CH₃ | |
| 438 | Cl | CN | COO—C(CH₃)₂COO—CH₂CH═CH₂ | |
| 439 | Cl | CN | COO—C(CH₃)₂COO—CH₂C≡CH | |
| 440 | Cl | CN | COO—C(CH₃)₂COO—CH₂CH₂OCH₃ | |
| 441 | Cl | CN | CONH₂ | |
| 442 | Cl | CN | CONHCH₃ | |
| 443 | Cl | CN | CON(CH₃)₂ | |
| 444 | Cl | CN | CONH—CH₂COO—CH₃ | |
| 445 | Cl | CN | CONH—CH₂COO—CH₂CH═CH₂ | |
| 446 | Cl | CN | CONH—CH₂COO—CH₂CH₂OCH₃ | |
| 447 | Cl | CN | CONH—CH(CH₃)COO—CH₃ | |
| 448 | Cl | CN | CONH—CH(CH₃)COO—CH₂CH═CH₂ | |
| 449 | Cl | CN | CONH—CH(CH₃)COO—CH₂CH₂OCH₃ | |
| 450 | Cl | CN | CON(CH₃)—CH₂COO—CH₃ | |
| 451 | Cl | CN | CON(CH₃)—CH₂COO—CH₂CH═CH₂ | |
| 452 | Cl | CN | CON(CH₃)—CH₂COO—CH₂CH₂OCH₃ | |
| 453 | Cl | CN | C(═N—OCH₃)O—CH₃ | |
| 454 | Cl | CN | C(═N—OCH₃)O—CH₂—COOCH₃ | |
| 455 | Cl | CN | C(═N—OCH₃)O—CH₂—COO-phenyl | |
| 456 | Cl | CN | C(═N—OCH₃)O—CH(CH₃)—COOCH₃ | |
| 457 | Cl | CN | CH═C(Cl)COO—CH₃ | |
| 458 | Cl | CN | CH═C(Cl)COO—CH₂CH₃ | |
| 459 | Cl | CN | CH═C(Cl)COO—CH₂CH═CH₂ | |
| 460 | Cl | CN | CH═C(Cl)COO—CH₂COOCH₃ | |
| 461 | Cl | CN | CH═C(Cl)COO—CH(CH₃)COOCH₃ | |
| 462 | Cl | CN | CH═C(Cl)CON(CH₃)₂ | |
| 463 | Cl | CN | CH═C(Cl)CON(CH₃)—CH₂COOCH₃ | |
| 464 | Cl | CN | CH═C(Cl)CONH—CH(CH₃)COOCH₃ | |
| 465 | Cl | CN | CH═C(Br)COO—CH₃ | |
| 466 | Cl | CN | CH═C(Br)COO—CH₂CH₃ | |
| 467 | Cl | CN | CH═C(CH₃)COO—CH₃ | |
| 468 | Cl | CN | CH═C(CH₃)COO—CH₂CH₃ | |
| 469 | Cl | CN | CH₂—CH(Cl)—COO—CH₃ | |
| 470 | Cl | CN | CH₂—CH(Cl)—COO—CH₂CH₃ | |
| 471 | Cl | CN | CHO | |
| 472 | Cl | CN | CH═N—OCH₃ | |
| 473 | Cl | CN | CH═N—OCH₂CH₃ | |
| 474 | Cl | CN | CH═N—OCH(CH₃)COOCH₃ | |
| 475 | Cl | CN | SO₂Cl | |
| 476 | Cl | CN | SO₂NH₂ | |
| 477 | Cl | CN | SO₂NHCH₃ | |
| 478 | Cl | CN | SO₂N(CH₃)₂ | |
| 479 | Cl | CN | NH—CH₂C≡CH | |
| 480 | Cl | CN | NHCH(CH₃)COOCH₃ | |
| 481 | Cl | CN | N(CH₃)—CH₂C≡CH | |
| 482 | Cl | CN | NH(SO₂CH₃) | |
| 483 | Cl | CN | N(CH₃)(SO₂CH₃) | |
| 484 | Cl | CN | N(SO₂CH₃)₂ | |
| 485 | H | Cl | H | |
| 486 | H | Cl | F | |
| 487 | H | Cl | CH₃ | |
| 488 | H | Cl | NO₂ | |
| 489 | H | Cl | NH₂ | |
| 490 | H | Cl | OH | |
| 491 | H | Cl | OCH₃ | |
| 492 | H | Cl | OCH(CH₃)₂ | |
| 493 | H | Cl | O—CH₂CH═CH₂ | |
| 494 | H | Cl | O—CH₂C≡CH | |
| 495 | H | Cl | O—CH(CH₃)C≡CH | |
| 496 | H | Cl | O-cyclopentyl | |
| 497 | H | Cl | OCH₂COOH | |
| 498 | H | Cl | OCH₂COO—CH₃ | |
| 499 | H | Cl | OCH₂COO—CH₂CH₃ | |
| 500 | H | Cl | OCH₂COO—CH₂CH═CH₂ | |
| 501 | H | Cl | OCH₂COO—CH₂C≡CH | |
| 502 | H | Cl | OCH₂COO—CH₂CH₂OCH₃ | |
| 503 | H | Cl | OCH₂CONH—CH₃ | |
| 504 | H | Cl | OCH₂CON(CH₃)₂ | |
| 505 | H | Cl | OCH(CH₃)COOH | |
| 506 | H | Cl | OCH(CH₃)COO—CH₃ | |
| 507 | H | Cl | OCH(CH₃)COO—CH₂CH₃ | |
| 508 | H | Cl | OCH(CH₃)COO—CH₂CH═CH₂ | |
| 509 | H | Cl | OCH(CH₃)COO—CH₂C≡CH | |
| 510 | H | Cl | OCH(CH₃)COO—CH₂CH₂OCH₃ | |
| 511 | H | Cl | OCH(CH₃)CONH—CH₃ | |
| 512 | H | Cl | OCH(CH₃)CON(CH₃)₂ | |
| 513 | H | Cl | OC(CH₃)₂COO—CH₃ | |
| 514 | H | Cl | OC(CH₃)₂COO—CH₂CH═CH₂ | |
| 515 | H | Cl | SH | |
| 516 | H | Cl | SCH₃ | |
| 517 | H | Cl | SCH(CH₃)₂ | |
| 518 | H | Cl | S—CH₂CH═CH₂ | |
| 519 | H | Cl | S—CH₂C≡CH | |
| 520 | H | Cl | S—CH(CH₃)C≡CH | |
| 521 | H | Cl | S-cyclopentyl | |
| 522 | H | Cl | SCH₂COOH | |
| 523 | H | Cl | SCH₂COO—CH₃ | |
| 524 | H | Cl | SCH₂COO—CH₂CH₃ | |
| 525 | H | Cl | SCH₂COO—CH₂CH═CH₂ | |
| 526 | H | Cl | SCH₂COO—CH₂C≡CH | |
| 527 | H | Cl | SCH₂COO—CH₂CH₂OCH₃ | |
| 528 | H | Cl | SCH₂CONH—CH₃ | |
| 529 | H | Cl | SCH₂CON(CH₃)₂ | |
| 530 | H | Cl | SCH(CH₃)COOH | |
| 531 | H | Cl | SCH(CH₃)COO—CH₃ | |
| 532 | H | Cl | SCH(CH₃)COO—CH₂CH₃ | |
| 533 | H | Cl | SCH(CH₃)COO—CH₂CH═CH₂ | |
| 534 | H | Cl | SCH(CH₃)COO—CH₂C≡CH | |
| 535 | H | Cl | SCH(CH₃)COO—CH₂CH₂OCH₃ | |
| 536 | H | Cl | SCH(CH₃)CONH—CH₃ | |
| 537 | H | Cl | SCH(CH₃)CON(CH₃)₂ | |
| 538 | H | Cl | SC(CH₃)₂COO—CH₃ | |
| 539 | H | Cl | SC(CH₃)₂COO—CH₂CH═CH₂ | |
| 540 | H | Cl | COOH | |
| 541 | H | Cl | COOCH₃ | |
| 542 | H | Cl | COOCH₂CH₃ | |
| 543 | H | Cl | COOCH(CH₃)₂ | |
| 544 | H | Cl | COO—CH₂CH═CH₂ | |
| 545 | H | Cl | COO—CH₂C≡CH | |
| 546 | H | Cl | COO-cyclopentyl | |
| 547 | H | Cl | COO—CH₂COO—CH₃ | |
| 548 | H | Cl | COO—CH₂COO—CH₂CH₃ | |
| 549 | H | Cl | COO—CH₂COO—CH₂CH═CH₂ | |
| 550 | H | Cl | COO—CH₂COO—CH₂C≡CH | |
| 551 | H | Cl | COO—CH₂COO—CH₂CH₂OCH₃ | |
| 552 | H | Cl | COO—CH(CH₃)COO—CH₃ | |
| 553 | H | Cl | COO—CH(CH₃)COO—CH₂CH₃ | |
| 554 | H | Cl | COO—CH(CH₃)COO—CH₂CH═CH₂ | |
| 555 | H | Cl | COO—CH(CH₃)COO—CH₂C≡CH | |
| 556 | H | Cl | COO—CH(CH₃)COO—CH₂CH₂OCH₃ | |
| 557 | H | Cl | COO—C(CH₃)₂COO—CH₃ | |
| 558 | H | Cl | COO—C(CH₃)₂COO—CH₂CH₃ | |
| 559 | H | Cl | COO—C(CH₃)₂COO—CH₂CH═CH₂ | |
| 560 | H | Cl | COO—C(CH₃)₂COO—CH₂C≡CH | |
| 561 | H | Cl | COO—C(CH₃)₂COO—CH₂CH₂OCH₃ | |
| 562 | H | Cl | CONH₂ | |
| 563 | H | Cl | CONHCH₃ | |
| 564 | H | Cl | CON(CH₃)₂ | |
| 565 | H | Cl | CONH—CH₂COO—CH₃ | |
| 566 | H | Cl | CONH—CH₂COO—CH₂CH═CH₂ | |
| 567 | H | Cl | CONH—CH₂COO—CH₂CH₂OCH₃ | |
| 568 | H | Cl | CONH—CH(CH₃)COO—CH₃ | |
| 569 | H | Cl | CONH—CH(CH₃)COO—CH₂CH═CH₂ | |
| 570 | H | Cl | CONH—CH(CH₃)COO—CH₂CH₂OCH₃ | |
| 571 | H | Cl | CON(CH₃)—CH₂COO—CH₃ | |
| 572 | H | Cl | CON(CH₃)—CH₂COO—CH₂CH═CH₂ | |
| 573 | H | Cl | CON(CH₃)—CH₂COO—CH₂CH₂OCH₃ | |
| 574 | H | Cl | C(═N—OCH₃)O—CH₃ | |
| 575 | H | Cl | C(═N—OCH₃)O—CH₂—COOCH₃ | |
| 576 | H | Cl | C(═N—OCH₃)O—CH₂—COO-phenyl | |
| 577 | H | Cl | C(═N—OCH₃)O—CH(CH₃)—COOCH₃ | |
| 578 | H | Cl | CH═C(Cl)COO—CH₃ | |
| 579 | H | Cl | CH═C(Cl)COO—CH₂CH₃ | |
| 580 | H | Cl | CH═C(Cl)COO—CH₂CH═CH₂ | |
| 581 | H | Cl | CH═C(Cl)COO—CH₂COOCH₃ | |
| 582 | H | Cl | CH═C(Cl)COO—CH(CH₃)COOCH₃ | |
| 583 | H | Cl | CH═C(Cl)CON(CH₃)₂ | |
| 584 | H | Cl | CH═C(Cl)CON(CH₃)—CH₂COOCH₃ | |
| 585 | H | Cl | CH═C(Cl)CONH—CH(CH₃)COOCH₃ | |
| 586 | H | Cl | CH═C(Br)COO—CH₃ | |
| 587 | H | Cl | CH═C(Br)COO—CH₂CH₃ | |
| 588 | H | Cl | CH═C(CH₃)COO—CH₃ | |
| 589 | H | Cl | CH═C(CH₃)COO—CH₂CH₃ | |
| 590 | H | Cl | CH₂—CH(Cl)—COO—CH₃ | |
| 591 | H | Cl | CH₂—CH(Cl)—COO—CH₂CH₃ | |
| 592 | H | Cl | CHO | |
| 593 | H | Cl | CH═N—OCH₃ | |
| 594 | H | Cl | CH═N—OCH₂CH₃ | |
| 595 | H | Cl | CH═N—OCH(CH₃)COOCH₃ | |
| 596 | H | Cl | SO₂Cl | |
| 597 | H | Cl | SO₂NH₂ | |
| 598 | H | Cl | SO₂NHCH₃ | |
| 599 | H | Cl | SO₂N(CH₃)₂ | |
| 600 | H | Cl | NH—CH₂C≡CH | |
| 601 | H | Cl | NHCH(CH₃)COOCH₃ | |
| 602 | H | Cl | N(CH₃)—CH₂C≡CH | |
| 603 | H | Cl | NH(SO₂CH₃) | |
| 604 | H | Cl | N(CH₃)(SO₂CH₃) | |
| 605 | H | Cl | N(SO₂CH₃)₂ | |
| 606 | H | CN | H | |
| 607 | H | CN | F | |
| 608 | H | CN | CH₃ | |
| 609 | H | CN | NO₂ | |
| 610 | H | CN | NH₂ | |
| 611 | H | CN | OH | |
| 612 | H | CN | OCH₃ | |
| 613 | H | CN | OCH(CH₃)₂ | |
| 614 | H | CN | O—CH₂CH═CH₂ | |
| 615 | H | CN | O—CH₂C≡CH | |
| 616 | H | CN | O—CH(CH₃)C≡CH | |
| 617 | H | CN | O-cyclopentyl | |
| 618 | H | CN | OCH₂COOH | |
| 619 | H | CN | OCH₂COO—CH₃ | |
| 620 | H | CN | OCH₂COO—CH₂CH₃ | |
| 621 | H | CN | OCH₂COO—CH₂CH═CH₂ | |
| 622 | H | CN | OCH₂COO—CH₂C≡CH | |
| 623 | H | CN | OCH₂COO—CH₂CH₂OCH₃ | |
| 624 | H | CN | OCH₂CONH—CH₃ | |
| 625 | H | CN | OCH₂CON(CH₃)₂ | |
| 626 | H | CN | OCH(CH₃)COOH | |
| 627 | H | CN | OCH(CH₃)COO—CH₃ | |
| 628 | H | CN | OCH(CH₃)COO—CH₂CH₃ | |
| 629 | H | CN | OCH(CH₃)COO—CH₂CH═CH₃ | |
| 630 | H | CN | OCH(CH₃)COO—CH₂C≡CH | |
| 631 | H | CN | OCH(CH₃)COO—CH₂CH₂OCH₃ | |
| 632 | H | CN | OCH(CH₃)CONH—CH₃ | |
| 633 | H | CN | OCH(CH₃)CON(CH₃)₂ | |
| 634 | H | CN | OC(CH₃)₂COO—CH₃ | |
| 635 | H | CN | OC(CH₃)₂COO—CH₂CH═CH₂ | |
| 636 | H | CN | SH | |
| 637 | H | CN | SCH₃ | |
| 638 | H | CN | SCH(CH₃)₂ | |
| 639 | H | CN | S—CH₂CH═CH₂ | |
| 640 | H | CN | S—CH₂C≡CH | |
| 641 | H | CN | S—CH(CH₃)C≡CH | |
| 642 | H | CN | S-cyclopentyl | |
| 643 | H | CN | SCH₂COOH | |
| 644 | H | CN | SCH₂COO—CH₃ | |
| 645 | H | CN | SCH₂COO—CH₂CH₃ | |
| 646 | H | CN | SCH₂COO—CH₂CH═CH₂ | |
| 647 | H | CN | SCH₂COO—CH₂C≡CH | |
| 648 | H | CN | SCH₂COO—CH₂CH₂OCH₃ | |
| 649 | H | CN | SCH₂CONH—CH₃ | |
| 650 | H | CN | SCH₂CON(CH₃)₂ | |
| 651 | H | CN | SCH(CH₃)COOH | |
| 652 | H | CN | SCH(CH₃)COO—CH₃ | |
| 653 | H | CN | SCH(CH₃)COO—CH₂CH₃ | |
| 654 | H | CN | SCH(CH₃)COO—CH₂CH═CH₂ | |
| 655 | H | CN | SCH(CH₃)COO—CH₂C≡CH | |
| 656 | H | CN | SCH(CH₃)COO—CH₂CH₂OCH₃ | |
| 657 | H | CN | SCH(CH₃)CONH—CH₃ | |
| 658 | H | CN | SCH(CH₃)CON(CH₃)₂ | |
| 659 | H | CN | SC(CH₃)₂COO—CH₃ | |
| 660 | H | CN | SC(CH₃)₂COO—CH₂CH═CH₂ | |
| 661 | H | CN | COOH | |
| 662 | H | CN | COOCH₃ | |
| 663 | H | CN | COOCH₂CH₃ | |
| 664 | H | CN | COOCH(CH₃)₂ | |
| 665 | H | CN | COO—CH₂CH═CH₂ | |
| 666 | H | CN | COO—CH₂C≡CH | |
| 667 | H | CN | COO-cyclopentyl | |
| 668 | H | CN | COO—CH₂COO—CH₃ | |
| 669 | H | CN | COO—CH₂COO—CH₂CH₃ | |
| 670 | H | CN | COO—CH₂COO—CH₂CH═CH₂ | |
| 671 | H | CN | COO—CH₂COO—CH₂C≡CH | |
| 672 | H | CN | COO—CH₂COO—CH₂CH₂OCH₃ | |
| 673 | H | CN | COO—CH(CH₃)COO—CH₃ | |
| 674 | H | CN | COO—CH(CH₃)COO—CH₂CH₃ | |
| 675 | H | CN | COO—CH(CH₃)COO—CH₂CH═CH₂ | |
| 676 | H | CN | COO—CH(CH₃)COO—CH₂C≡CH | |
| 677 | H | CN | COO—CH(CH₃)COO—CH₂CH₂OCH₃ | |
| 678 | H | CN | COO—C(CH₃)₂COO—CH₃ | |
| 679 | H | CN | COO—C(CH₃)₂COO—CH₂CH₃ | |
| 680 | H | CN | COO—C(CH₃)₂COO—CH₂CH═CH₂ | |
| 681 | H | CN | COO—C(CH₃)₂COO—CH₂C≡CH | |
| 682 | H | CN | COO—C(CH₃)₂COO—CH₂CH₂OCH₃ | |
| 683 | H | CN | CONH₂ | |
| 684 | H | CN | CONHCH₃ | |
| 685 | H | CN | CON(CH₃)₂ | |
| 686 | H | CN | CONH—CH₂COO—CH₃ | |
| 687 | H | CN | CONH—CH₂COO—CH₂CH═CH₂ | |
| 688 | H | CN | CONH—CH₂COO—CH₂CH₂OCH₃ | |
| 689 | H | CN | CONH—CH(CH₃)COO—CH₃ | |
| 690 | H | CN | CONH—CH(CH₃)COO—CH₂CH═CH₂ | |
| 691 | H | CN | CONH—CH(CH₃)COO—CH₂CH₂OCH₃ | |
| 692 | H | CN | CON(CH₃)—CH₂COO—CH₃ | |
| 693 | H | CN | CON(CH₃)—CH₂COO—CH₂CH═CH₂ | |
| 694 | H | CN | CON(CH₃)—CH₂COO—CH₂CH₂OCH₃ | |
| 695 | H | CN | C(═N—OCH₃)O—CH₃ | |
| 696 | H | CN | C(═N—OCH₃)O—CH₂—COOCH₃ | |
| 697 | H | CN | C(═N—OCH₃)O—CH₂—COO-phenyl | |
| 698 | H | CN | C(═N—OCH₃)O—CH(CH₃)—COOCH₃ | |
| 699 | H | CN | CH═C(Cl)COO—CH₃ | |
| 700 | H | CN | CH═C(Cl)COO—CH₂CH₃ | |
| 701 | H | CN | CH═C(Cl)COO—CH₂CH═CH₂ | |
| 702 | H | CN | CH═C(Cl)COO—CH₂COOCH₃ | |
| 703 | H | CN | CH═C(Cl)COO—CH(CH₃)COOCH₃ | |
| 704 | H | CN | CH═C(Cl)CON(CH₃)₂ | |
| 705 | H | CN | CH═C(Cl)CON(CH₃)—CH₂COOCH₃ | |
| 706 | H | CN | CH═C(Cl)CONH—CH(CH₃)COOCH₃ | |
| 707 | H | CN | CH═C(Br)COO—CH₃ | |
| 708 | H | CN | CH═C(Br)COO—CH₂CH₃ | |
| 709 | H | CN | CH═C(CH₃)COO—CH₃ | |
| 710 | H | CN | CH═C(CH₃)COO—CH₂CH₃ | |
| 711 | H | CN | CH₂—CH(Cl)—COO—CH₃ | |
| 712 | H | CN | CH₂—CH(Cl)—COO—CH₂CH₃ | |
| 713 | H | CN | CHO | |
| 714 | H | CN | CH═N—OCH₃ | |
| 715 | H | CN | CH═N—OCH₂CH₃ | |
| 716 | H | CN | CH═N—OCH(CH₃)COOCH₃ | |
| 717 | H | CN | SO₂Cl | |
| 718 | H | CN | SO₂NH₂ | |
| 719 | H | CN | SO₂NHCH₃ | |
| 720 | H | CN | SO₂N(CH₃)₂ | |
| 721 | H | CN | NH—CH₂C≡CH | |
| 722 | H | CN | NHCH(CH₃)COOCH₃ | |
| 723 | H | CN | N(CH₃)—CH₂C≡CH | |
| 724 | H | CN | NH(SO₂CH₃) | |
| 725 | H | CN | N(CH₃)(SO₂CH₃) | |
| 726 | H | CN | N(SO₂CH₃)₂ | |
| 727 | F | Cl | OCH(CH₃)COO—CH₃ | (R |
| | | | | enantiomer) |
| 728 | F | Cl | OCH(CH₃)COO—CH₂CH₃ | (R |
| | | | | enantiomer) |
| 729 | F | Cl | OCH(CH₃)COO—CH₂CH═CH₂ | (R |
| | | | | enantiomer) |
| 730 | F | Cl | OCH(CH₃)COO—CH₂C≡CH | (R |
| | | | | enantiomer) |
| 731 | F | Cl | OCH(CH₃)COO—CH₂CH₂OCH₃ | (R |
| | | | | enantiomer) |
| 732 | F | Cl | OCH(CH₃)CONH—CH₃ | (R |
| | | | | enantiomer) |
| 733 | F | Cl | OCH(CH₃)CON(CH₃)₂ | (R |
| | | | | enantiomer) |
| 734 | F | CN | OCH(CH₃)COO—CH₃ | (R |
| | | | | enantiomer) |
| 735 | F | CN | OCH(CH₃)COO—CH₂CH₃ | (R |
| | | | | enantiomer) |
| 736 | F | CN | OCH(CH₃)COO—CH₂CH═CH₂ | (R |
| | | | | enantiomer) |
| 737 | F | CN | OCH(CH₃)COO—CH₂C≡CH | (R |
| | | | | enantiomer) |
| 738 | F | CN | OCH(CH₃)COO—CH₂CH₂OCH₃ | (R |
| | | | | enantiomer) |
| 739 | F | CN | OCH(CH₃)CONH—CH₃ | (R |
| | | | | enantiomer) |
| 740 | F | CN | OCH(CH₃)CON(CH₃)₂ | (R |
| | | | | enantiomer) |
| 741 | H | Cl | OCH(CH₃)COO—CH₃ | (R |
| | | | | enantiomer) |
| 742 | H | Cl | OCH(CH₃)COO—CH₂CH₃ | (R |
| | | | | enantiomer) |
| 743 | H | Cl | OCH(CH₃)COO—CH₂CH═CH₂ | (R |
| | | | | enantiomer) |
| 744 | H | Cl | OCH(CH₃)COO—CH₂C≡CH | (R |
| | | | | enantiomer) |
| 745 | H | Cl | OCH(CH₃)COO—CH₂CH₂OCH₃ | (R |
| | | | | enantiomer) |
| 746 | H | Cl | OCH(CH₃)CONH—CH₃ | (R |
| | | | | enantiomer) |
| 747 | H | Cl | OCH(CH₃)CON(CH₃)₂ | (R |
| | | | | enantiomer) |
| 748 | H | CN | OCH(CH₃)COO—CH₃ | (R |
| | | | | enantiomer) |
| 749 | H | CN | OCH(CH₃)COO—CH₂CH₃ | (R |
| | | | | enantiomer) |
| 750 | H | CN | OCH(CH₃)COO—CH₂CH═CH₂ | (R |
| | | | | enantiomer) |
| 751 | H | CN | OCH(CH₃)COO—CH₂C≡CH | (R |
| | | | | enantiomer) |
| 752 | H | CN | OCH(CH₃)COO—CH₂CH₂OCH₃ | (R |
| | | | | enantiomer) |
| 753 | H | CN | OCH(CH₃)CONH—CH₃ | (R |
| | | | | enantiomer) |
| 754 | H | CN | OCH(CH₃)CON(CH₃)₂ | (R |
| | | | | enantiomer) |
| 755 | Cl | Cl | OCH(CH₃)COO—CH₃ | (R |
| | | | | enantiomer) |
| 756 | Cl | Cl | OCH(CH₃)COO—CH₂CH₃ | (R |
| | | | | enantiomer) |
| 757 | Cl | Cl | OCH(CH₃)COO—CH₂CH═CH₂ | (R |
| | | | | enantiomer) |
| 758 | Cl | Cl | OCH(CH₃)COO—CH₂C≡CH | (R |
| | | | | enantiomer) |
| 759 | Cl | Cl | OCH(CH₃)COO—CH₂CH₂OCH₃ | (R |
| | | | | enantiomer) |
| 760 | Cl | Cl | OCH(CH₃)CONH—CH₃ | (R |
| | | | | enantiomer) |
| 761 | Cl | Cl | OCH(CH₃)CON(CH₃)₂ | (R |
| | | | | enantiomer) |
| 762 | Cl | CN | OCH(CH₃)COO—CH₃ | (R |
| | | | | enantiomer) |
| 763 | Cl | CN | OCH(CH₃)COO—CH₂CH₃ | (R |
| | | | | enantiomer) |
| 764 | Cl | CN | OCH(CH₃)COO—CH₂CH═CH₂ | (R |
| | | | | enantiomer) |
| 765 | Cl | CN | OCH(CH₃)COO—CH₂C≡CH | (R |
| | | | | enantiomer) |
| 766 | Cl | CN | OCH(CH₃)COO—CH₂CH₂OCH₃ | (R |
| | | | | enantiomer) |
| 767 | Cl | CN | OCH(CH₃)CONH—CH₃ | (R |
| | | | | enantiomer) |
| 768 | Cl | CN | OCH(CH₃)CON(CH₃)₂ | (R |
| | | | | enantiomer) |
| 769 | F | Cl | COO—CH(CH₃)COO—CH₃ | (S |
| | | | | enantiomer) |
| 770 | F | Cl | COO—CH(CH₃)COO—CH₂CH₃ | (S |
| | | | | enantiomer) |
| 771 | F | Cl | COO—CH(CH₃)COO—CH₂CH═CH₂ | (S |
| | | | | enantiomer) |
| 772 | F | Cl | COO—CH(CH₃)COO—CH₂C≡CH | (S |
| | | | | enantiomer) |
| 773 | F | Cl | COO—CH(CH₃)COO—CH₂CH₂OCH₃ | (S |
| | | | | enantiomer) |
| 774 | F | CN | COO—CH(CH₃)COO—CH₃ | (S |
| | | | | enantiomer) |
| 775 | F | CN | COO—CH(CH₃)COO—CH₂CH₃ | (S |
| | | | | enantiomer) |
| 776 | F | CN | COO—CH(CH₃)COO—CH₂CH═CH₂ | (S |
| | | | | enantiomer) |
| 777 | F | CN | COO—CH(CH₃)COO—CH₂C≡CH | (S |
| | | | | enantiomer) |
| 778 | F | CN | COO—CH(CH₃)COO—CH₂CH₂OCH₃ | (S |
| | | | | enantiomer) |
| 779 | Cl | Cl | COO—CH(CH₃)COO—CH₃ | (S |
| | | | | enantiomer) |
| 780 | Cl | Cl | COO—CH(CH₃)COO—CH₂CH₃ | (S |
| | | | | enantiomer) |
| 781 | Cl | Cl | COO—CH(CH₃)COO—CH₂CH═CH₂ | (S |
| | | | | enantiomer) |
| 782 | Cl | Cl | COO—CH(CH₃)COO—CH₂C≡CH | (S |
| | | | | enantiomer) |
| 783 | Cl | Cl | COO—CH(CH₃)COO—CH₂CH₂OCH₃ | (S |
| | | | | enantiomer) |
| 784 | Cl | CN | COO—CH(CH₃)COO—CH₃ | (S |
| | | | | enantiomer) |
| 785 | Cl | CN | COO—CH(CH₃)COO—CH₂CH₃ | (S |
| | | | | enantiomer) |
| 786 | Cl | CN | COO—CH(CH₃)COO—CH₂CH═CH₂ | (S |
| | | | | enantiomer) |
| 787 | Cl | CN | COO—CH(CH₃)COO—CH₂C≡CH | (S |
| | | | | enantiomer) |
| 788 | Cl | CN | COO—CH(CH₃)COO—CH₂CH₂OCH₃ | (S |
| | | | | enantiomer) |
| 789 | H | Cl | COO—CH(CH₃)COO—CH₃ | (S |
| | | | | enantiomer) |
| 790 | H | Cl | COO—CH(CH₃)COO—CH₂CH₃ | (S |
| | | | | enantiomer) |
| 791 | H | Cl | COO—CH(CH₃)COO—CH₂CH═CH₂ | (S |
| | | | | enantiomer) |
| 792 | H | Cl | COO—CH(CH₃)COO—CH₂C≡CH | (S |
| | | | | enantiomer) |
| 793 | H | Cl | COO—CH(CH₃)COO—CH₂CH₂OCH₃ | (S |
| | | | | enantiomer) |
| 794 | H | CN | COO—CH(CH₃)COO—CH₃ | (S |
| | | | | enantiomer) |
| 795 | H | CN | COO—CH(CH₃)COO—CH₂CH₃ | (S |
| | | | | enantiomer) |
| 796 | H | CN | COO—CH(CH₃)COO—CH₂CH═CH₂ | (S |
| | | | | enantiomer) |
| 797 | H | CN | COO—CH(CH₃)COO—CH₂C≡CH | (S |
| | | | | enantiomer) |
| 798 | H | CN | COO—CH(CH₃)COO—CH₂CH₂OCH₃ | (S |
| | | | | enantiomer) |

Among the compounds IB (Q=N), preference is given to the compounds where R³=CF₃ and R¹=Cl in which R² and R^{2′} independently of one another are selected from the group consisting of hydrogen and methyl. Examples of these are the compounds of the formulae IBa, IBb, IBc and IBd given below in which R⁴, R⁵ and X—R⁶ together in each case have the meanings given in one row of Table 1 (compounds IBa.1-IBa.798 to IBd.1-IBd.798).

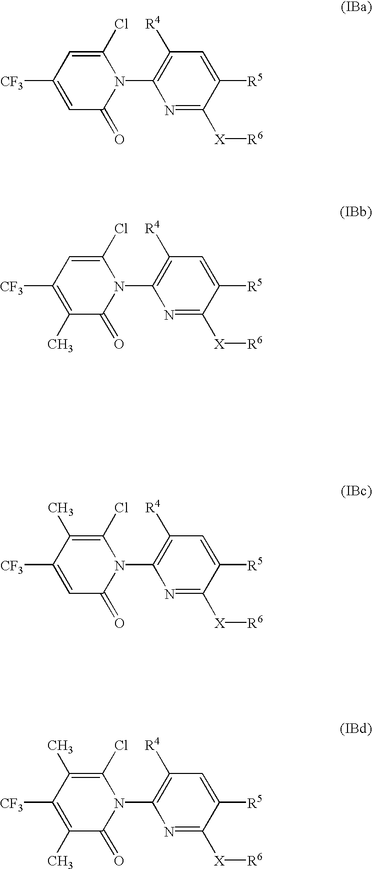

Among the compounds IC, particular preference is given to those compounds in which R⁷ together with X—R⁶ is a chain of the formula N═C(R¹⁹)—O— or N═C(R¹⁹)—S— in which the variable R¹⁹ has the meanings given above, in particular the meanings given as being preferred. Hereinbelow, these compounds are also referred to as benzoxazolylpyridones or as benzothiazolylpyridones. Preference is given here to those compounds in which the chalcogen atom is attached to the carbon atom which is adjacent to the point of attachment of the pyridone ring.

Among these compounds, in turn, preference is given to those compounds where R³=CF₃ and R¹=Cl in which R² and R^{2′} independently of one another are selected from the group consisting of hydrogen and methyl.

Examples of these are the 1-benzoxazol-7-yl-1H-2-pyridones of the formulae ICa, ICb, ICc and ICd in which R⁴, R⁵ and R¹⁹ together in each case have the meanings given in one row of Table 2 (compounds ICa.1-ICa.312 to ICd.1-ICd.312).

TABLE 2

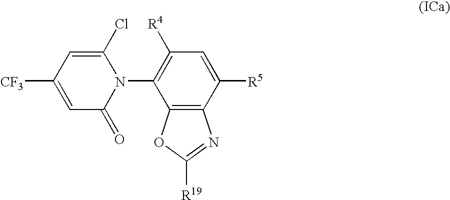

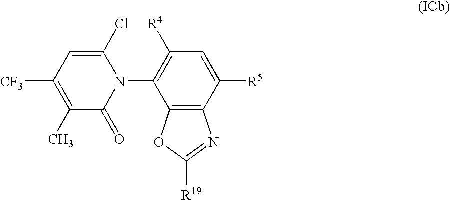

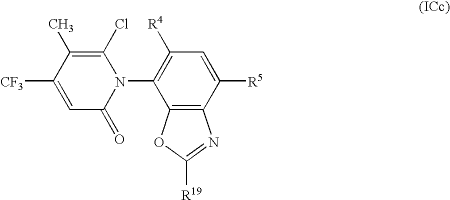

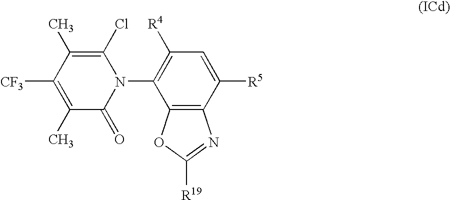

| No. | R⁴ | R⁵ | R¹⁹ |
|---|---|---|---|
| 1 | F | Cl | H |
| 2 | F | Cl | CH₃ |
| 3 | F | Cl | C₂H₅ |
| 4 | F | Cl | n-C₃H₇ |
| 5 | F | Cl | CH(CH₃)₂ |
| 6 | F | Cl | n-C₄H₉ |
| 7 | F | Cl | CH(CH₃)—C₂H₅ |
| 8 | F | Cl | CH₂—CH(CH₃)₂ |
| 9 | F | Cl | C(CH₃)₃ |
| 10 | F | Cl | CH₂—CH═CH₂ |
| 11 | F | Cl | CH₂—C≡CH |
| 12 | F | Cl | CH₂Cl |
| 13 | F | Cl | CF₃ |
| 14 | F | Cl | CH₂-cyclopropyl |
| 15 | F | Cl | cyclopropyl |
| 16 | F | Cl | cyclopentyl |
| 17 | F | Cl | cyclohexyl |
| 18 | F | Cl | tetrahydropyran-3-yl |
| 19 | F | Cl | tetrahydropyran-4-yl |
| 20 | F | Cl | tetrahydrothiopyran-3-yl |
| 21 | F | Cl | tetrahydrothiopyran-4-yl |
| 22 | F | Cl | phenyl |
| 23 | F | Cl | CH₂—COOCH₃ |
| 24 | F | Cl | CH₂—COOC₂H₅ |
| 25 | F | Cl | CH₂—CH₂—COOCH₃ |
| 26 | F | Cl | CH₂—CH₂—COOC₂H₅ |
| 27 | F | Cl | F |
| 28 | F | Cl | Cl |
| 29 | F | Cl | Br |
| 30 | F | Cl | OCH₃ |
| 31 | F | Cl | OCH₂CH₃ |
| 32 | F | Cl | O-n-C₃H₇ |
| 33 | F | Cl | OCH(CH₃)₂ |
| 34 | F | Cl | OCH₂—CH═CH₂ |
| 35 | F | Cl | OCH₂—C≡CH |
| 36 | F | Cl | OCH₂—COOCH₃ |
| 37 | F | Cl | OCH₂—COOC₂H₅ |
| 38 | F | Cl | OCH(CH₃)—COOCH₃ |
| 39 | F | Cl | OCH(CH₃)—COOC₂H₅ |
| 40 | F | Cl | NH₂ |
| 41 | F | Cl | N(CH₃)₂ |
| 42 | F | Cl | SCH₃ |
| 43 | F | Cl | SCH₂CH₃ |
| 44 | F | Cl | S-n-C₃H₇ |
| 45 | F | Cl | SCH(CH₃)₂ |
| 46 | F | Cl | SCH₂—CHCH₂ |
| 47 | F | Cl | SCH₂—C≡CH |
| 48 | F | Cl | SCH₂—COOCH₃ |
| 49 | F | Cl | SCH₂—COOC₂H₅ |
| 50 | F | Cl | SCH(CH₃)—COOCH₃ |
| 51 | F | Cl | COOCH₃ |
| 52 | F | Cl | COOC₂H₅ |
| 53 | Cl | Cl | H |
| 54 | Cl | Cl | CH₃ |
| 55 | Cl | Cl | C₂H₅ |
| 56 | Cl | Cl | n-C₃H₇ |
| 57 | Cl | Cl | CH(CH₃)₂ |
| 58 | Cl | Cl | n-C₄H₉ |
| 59 | Cl | Cl | CH(CH₃)—C₂H₅ |
| 60 | Cl | Cl | CH₂—CH(CH₃)₂ |
| 61 | Cl | Cl | C(CH₃)₃ |
| 62 | Cl | Cl | CH₂—CH═CH₂ |
| 63 | Cl | Cl | CH₂—C≡CH |
| 64 | Cl | Cl | CH₂Cl |
| 65 | Cl | Cl | CF₃ |
| 66 | Cl | Cl | CH₂-cyolopropyl |
| 67 | Cl | Cl | cyclopropyl |
| 68 | Cl | Cl | cyclopentyl |
| 69 | Cl | Cl | cyclohexyl |
| 70 | Cl | Cl | tetrahydropyran-3-yl |
| 71 | Cl | Cl | tetrahydropyran-4-yl |
| 72 | Cl | Cl | tetrahydrothiopyran-3-yl |
| 73 | Cl | Cl | tetrahydrothiopyran-4-yl |
| 74 | Cl | Cl | phenyl |
| 75 | Cl | Cl | CH₂—COOCH₃ |
| 76 | Cl | Cl | CH₂—COOC₂H₅ |
| 77 | Cl | Cl | CH₂—CH₂—COOCH₃ |
| 78 | Cl | Cl | CH₂—CH₂—COOC₂H₅ |
| 79 | Cl | Cl | F |
| 80 | Cl | Cl | Cl |
| 81 | Cl | Cl | Br |
| 82 | Cl | Cl | OCH₃ |
| 83 | Cl | Cl | OCH₂CH₃ |
| 84 | Cl | Cl | O-n-C₃H₇ |
| 85 | Cl | Cl | OCH(CH₃)₂ |
| 86 | Cl | Cl | OCH₂—CH═CH₂ |
| 87 | Cl | Cl | OCH₂—C≡CH |
| 88 | Cl | Cl | OCH₂—COOCH₃ |
| 89 | Cl | Cl | OCH₂—COOC₂H₅ |
| 90 | Cl | Cl | OCH(CH₃)—COOCH₃ |
| 91 | Cl | Cl | OCH(CH₃)—COOC₂H₅ |
| 92 | Cl | Cl | NH₂ |
| 93 | Cl | Cl | N(CH₃)₂ |
| 94 | Cl | Cl | SCH₃ |
| 95 | Cl | Cl | SCH₂CH₃ |
| 96 | Cl | Cl | S-n-C₃H₇ |
| 97 | Cl | Cl | SCH(CH₃)₂ |
| 98 | Cl | Cl | SCH₂—CH═CH₂ |
| 99 | Cl | Cl | SCH₂—C≡CH |
| 100 | Cl | Cl | SCH₂—COOCH₃ |
| 101 | Cl | Cl | SCH₂—COOC₂H₅ |
| 102 | Cl | Cl | SCH(CH₃)—COOCH₃ |
| 103 | Cl | Cl | COOCH₃ |
| 104 | Cl | Cl | COOC₂H₅ |
| 105 | H | Cl | H |
| 106 | H | Cl | CH₃ |
| 107 | H | Cl | C₂H₅ |
| 108 | H | Cl | n-C₃H₇ |
| 109 | H | Cl | CH(CH₃)₂ |
| 110 | H | Cl | n-C₄H₉ |
| 111 | H | Cl | CH(CH₃)—C₂H₅ |
| 112 | H | Cl | CH₂—CH(CH₃)₂ |
| 113 | H | Cl | C(CH₃)₃ |
| 114 | H | Cl | CH₂—CH═CH₂ |
| 115 | H | Cl | CH₂—C≡CH |
| 116 | H | Cl | CH₂Cl |
| 117 | H | Cl | CF₃ |
| 118 | H | Cl | CH₂-cyclopropyl |
| 119 | H | Cl | cyclopropyl |
| 120 | H | Cl | cyclopentyl |
| 121 | H | Cl | cyclohexyl |
| 122 | H | Cl | tetrahydropyran-3-yl |
| 123 | H | Cl | tetrahydropyran-4-yl |
| 124 | H | Cl | tetrahydrothiopyran-3-yl |
| 125 | H | Cl | tetrahydrothiopyran-4-yl |
| 126 | H | Cl | phenyl |
| 127 | H | Cl | CH₂—COOCH₃ |
| 128 | H | Cl | CH₂—COOC₂H₅ |
| 129 | H | Cl | CH₂—CH₂—COOCH₃ |
| 130 | H | Cl | CH₂—CH₂—COOC₂H₅ |
| 131 | H | Cl | F |
| 132 | H | Cl | Cl |
| 133 | H | Cl | Br |
| 134 | H | Cl | OCH₃ |
| 135 | H | Cl | OCH₂CH₃ |
| 136 | H | Cl | O-n-C₃H₇ |
| 137 | H | Cl | OCH(CH₃)₂ |
| 138 | H | Cl | OCH₂—CH═CH₂ |
| 139 | H | Cl | OCH₂—C≡CH |
| 140 | H | Cl | OCH₂—COOCH₃ |
| 141 | H | Cl | OCH₂—COOC₂H₅ |
| 142 | H | Cl | OCH(CH₃)—COOCH₃ |
| 143 | H | Cl | OCH(CH₃)—COOC₂H₅ |
| 144 | H | Cl | NH₂ |
| 145 | H | Cl | N(CH₃)₂ |
| 146 | H | Cl | SCH₃ |
| 147 | H | Cl | SCH₂CH₃ |
| 148 | H | Cl | S-n-C₃H₇ |
| 149 | H | Cl | SCH(CH₃)₂ |
| 150 | H | Cl | SCH₂—CH═CH₂ |
| 151 | H | Cl | SCH₂—C≡CH |
| 152 | H | Cl | SCH₂—COOCH₃ |
| 153 | H | Cl | SCH₂—COOC₂H₅ |
| 154 | H | Cl | SCH(CH₃)—COOCH₃ |
| 155 | H | Cl | COOCH₃ |
| 156 | H | Cl | COOC₂H₅ |
| 157 | F | CN | H |
| 158 | F | CN | CH₃ |
| 159 | F | CN | C₂H₅ |
| 160 | F | CN | n-C₃H₇ |
| 161 | F | CN | CH(CH₃)₂ |
| 162 | F | CN | n-C₄H₉ |
| 163 | F | CN | CH(CH₃)—C₂H₅ |
| 164 | F | CN | CH₂—CH(CH₃)₂ |
| 165 | F | CN | C(CH₃)₃ |
| 166 | F | CN | CH₂—CH═CH₂ |
| 167 | F | CN | CH₂—C≡CH |
| 168 | F | CN | CH₂Cl |
| 169 | F | CN | CF₃ |
| 170 | F | CN | CH₂-cyclopropyl |
| 171 | F | CN | cyclopropyl |
| 172 | F | CN | cyclopentyl |
| 173 | F | CN | cyclohexyl |
| 174 | F | CN | tetrahydropyran-3-yl |
| 175 | F | CN | tetrahydropyran-4-yl |
| 176 | F | CN | tetrahydrothiopyran-3-yl |
| 177 | F | CN | tetrahydrothiopyran-4-yl |
| 178 | F | CN | phenyl |
| 179 | F | CN | CH₂—COOCH₃ |
| 180 | F | CN | CH₂—COOC₂H₅ |
| 181 | F | CN | CH₂—CH₂—COOCH₃ |
| 182 | F | CN | CH₂—CH₂—COOC₂H₅ |
| 183 | F | CN | F |
| 184 | F | CN | Cl |
| 185 | F | CN | Br |
| 186 | F | CN | OCH₃ |
| 187 | F | CN | OCH₂CH₃ |
| 188 | F | CN | O-n-C₃H₇ |
| 189 | F | CN | OCH(CH₃)₂ |
| 190 | F | CN | OCH₂—CH═CH₂ |
| 191 | F | CN | OCH₂—C≡CH |
| 192 | F | CN | OCH₂—COOCH₃ |
| 193 | F | CN | OCH₂—COOC₂H₅ |
| 194 | F | CN | OCH(CH₃)—COOCH₃ |
| 195 | F | CN | OCH(CH₃)—COOC₂H₅ |
| 196 | F | CN | NH₂ |
| 197 | F | CN | N(CH₃)₂ |
| 198 | F | CN | SCH₃ |
| 199 | F | CN | SCH₂CH₃ |
| 200 | F | CN | S-n-C₃H₇ |
| 201 | F | CN | SCH(CH₃)₂ |
| 202 | F | CN | SCH₂—CH═CH₂ |
| 203 | F | CN | SCH₂—C≡CH |
| 204 | F | CN | SCH₂—COOCH₃ |
| 205 | F | CN | SCH₂—COOC₂H₅ |
| 206 | F | CN | SCH(CH₃)—COOCH₃ |
| 207 | F | CN | COOCH₃ |
| 208 | F | CN | COOC₂H₅ |
| 209 | Cl | CN | H |
| 210 | Cl | CN | CH₃ |
| 211 | Cl | CN | C₂H₅ |
| 212 | Cl | CN | n-C₃H₇ |
| 213 | Cl | CN | CH(CH₃)₂ |
| 214 | Cl | CN | n-C₄H₉ |
| 215 | Cl | CN | CH(CH₃)—C₂H₅ |
| 216 | Cl | CN | CH₂—CH(CH₃)₂ |
| 217 | Cl | CN | C(CH₃)₃ |
| 218 | Cl | CN | CH₂—CH═CH₂ |
| 219 | Cl | CN | CH₂—C≡CH |
| 220 | Cl | CN | CH₂Cl |
| 221 | Cl | CN | CF₃ |
| 222 | Cl | CN | CH₂-cyclopropyl |
| 223 | Cl | CN | cyclopropyl |
| 224 | Cl | CN | cyclopentyl |
| 225 | Cl | CN | cyclohexyl |
| 226 | Cl | CN | tetrahydropyran-3-yl |
| 227 | Cl | CN | tetrahydropyran-4-yl |
| 228 | Cl | CN | tetrahydrothiopyran-3-yl |
| 229 | Cl | CN | tetrahydrothiopyran-4-yl |
| 230 | Cl | CN | phenyl |
| 231 | Cl | CN | CH₂—COOCH₃ |
| 232 | Cl | CN | CH₂—COOC₂H₅ |
| 233 | Cl | CN | CH₂—CH₂—COOCH₃ |
| 234 | Cl | CN | CH₂—CH₂—COOC₂H₅ |
| 235 | Cl | CN | F |
| 236 | Cl | CN | Cl |
| 237 | Cl | CN | Br |
| 238 | Cl | CN | OCH₃ |
| 239 | Cl | CN | OCH₂CH₃ |
| 240 | Cl | CN | O-n-C₃H₇ |
| 241 | Cl | CN | OCH(CH₃)₂ |
| 242 | Cl | CN | OCH₂—CH═CH₂ |
| 243 | Cl | CN | OCH₂—C≡CH |
| 244 | Cl | CN | OCH₂—COOCH₃ |
| 245 | Cl | CN | OCH₂—COOC₂H₅ |
| 246 | Cl | CN | OCH(CH₃)—COOCH₃ |
| 247 | Cl | CN | OCH(CH₃)—COOC₂H₅ |
| 248 | Cl | CN | NH₂ |
| 249 | Cl | CN | N(CH₃)₂ |
| 250 | Cl | CN | SCH₃ |
| 251 | Cl | CN | SCH₂CH₃ |
| 252 | Cl | CN | S-n-C₃H₇ |
| 253 | Cl | CN | SCH(CH₃)₂ |
| 254 | Cl | CN | SCH₂—CH═CH₂ |
| 255 | Cl | CN | SCH₂—C≡CH |
| 256 | Cl | CN | SCH₂—COOCH₃ |
| 257 | Cl | CN | SCH₂—COOC₂H₅ |
| 258 | Cl | CN | SCH(CH₃)—COOCH₃ |
| 259 | Cl | CN | COOCH₃ |
| 260 | Cl | CN | COOC₂H₅ |
| 261 | H | CN | H |
| 262 | H | CN | CH₃ |
| 263 | H | CN | C₂H₅ |
| 264 | H | CN | n-C₃H₇ |
| 265 | H | CN | CH(CH₃)₂ |
| 266 | H | CN | n-C₄H₉ |
| 267 | H | CN | CH(CH₃)—C₂H₅ |
| 268 | H | CN | CH₂—CH(CH₃)₂ |
| 269 | H | CN | C(CH₃)₃ |
| 270 | H | CN | CH₂—CH═CH₂ |
| 271 | H | CN | CH₂—C≡CH |
| 272 | H | CN | CH₂Cl |
| 273 | H | CN | CF₃ |
| 274 | H | CN | CH₂-cyclopropyl |
| 275 | H | CN | cyclopropyl |
| 276 | H | CN | cyclopentyl |
| 277 | H | CN | cyclohexyl |
| 278 | H | CN | tetrahydropyran-3-yl |
| 279 | H | CN | tetrahydropyran-4-yl |
| 280 | H | CN | tetrahydrothiopyran-3-yl |
| 281 | H | CN | tetrahydrothiopyran-4-yl |
| 282 | H | CN | phenyl |
| 283 | H | CN | CH₂—COOCH₃ |
| 284 | H | CN | CH₂—COOC₂H₅ |
| 285 | H | CN | CH₂—CH₂—COOCH₃ |
| 286 | H | CN | CH₂—CH₂—COOC₂H₅ |
| 287 | H | CN | F |
| 288 | H | CN | Cl |
| 289 | H | CN | Br |
| 290 | H | CN | OCH₃ |
| 291 | H | CN | OCH₂CH₃ |
| 292 | H | CN | O-n-C₃H₇ |
| 293 | H | CN | OCH(CH₃)₂ |
| 294 | H | CN | OCH₂—CH═CH₂ |
| 295 | H | CN | OCH₂—C≡CH |
| 296 | H | CN | OCH₂—COOCH₃ |
| 297 | H | CN | OCH₂—COOC₂H₅ |
| 298 | H | CN | OCH(CH₃)—COOCH₃ |
| 299 | H | CN | OCH(CH₃)—COOC₂H₅ |
| 300 | H | CN | NH₂ |
| 301 | H | CN | N(CH₃)₂ |
| 302 | H | CN | SCH₃ |
| 303 | H | CN | SCH₂CH₃ |
| 304 | H | CN | S-n-C₃H₇ |
| 305 | H | CN | SCH(CH₃)₂ |
| 306 | H | CN | SCH₂—CH═CH₂ |
| 307 | H | CN | SCH₂—C≡CH |
| 308 | H | CN | SCH₂—COOCH₃ |
| 309 | H | CN | SCH₂—COOC₂H₅ |
| 310 | H | CN | SCH(CH₃)—COOCH₃ |
| 311 | H | CN | COOCH₃ |
| 312 | H | CN | COOC₂H₅ |

Examples of particularly preferred compounds IC include the 1-benzothiazol-7-yl-2-[1H]-pyridones of the formulae ICe, ICf, ICg and ICh given below in which R⁴, R⁵ and R¹⁹ together in each case have the meanings given in one row of Table 2 (compounds ICe.1-ICe.312 to ICh.1-ICh.312).

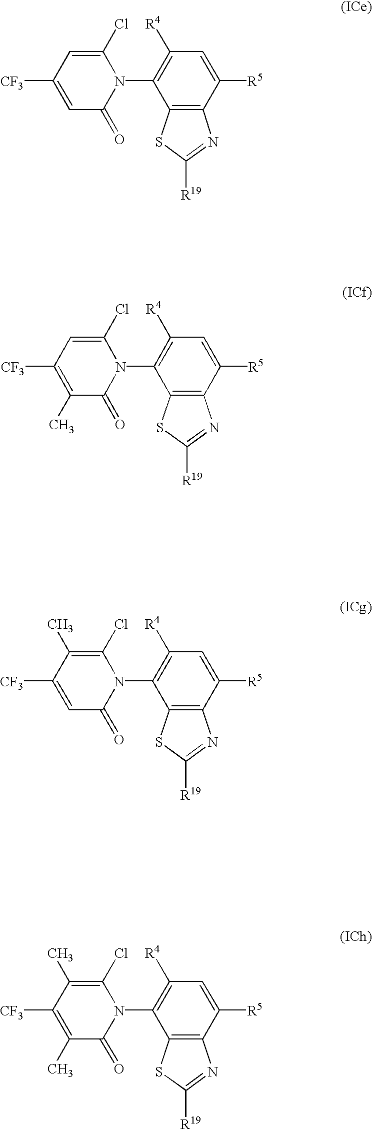

The 1-arylpyridones of the formula I according to the invention can be prepared similarly to known processes for the preparation of 1-arylpyridones and in particular by the synthesis routes described below. Hereinbelow, “aryl” denotes a radical of the formula:

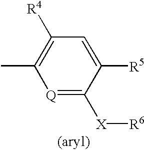

and “pyridonyl” denotes a radical of the formula:

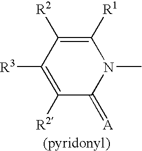

### A) Linking the Pyridone Unit to an Aromatic Compound Derived From the radical “Aryl”

A.1 Condensation of 1,5-Dicarboxylic Acids With Aryl Amines:

The preparation of 1-aryl-2-[1H]-pyridones of the formula I can be carried out, for example, by the synthesis route shown in scheme 1. Here, in a first step, a 3-haloalkyl-1,5-dicarboxylic acid or the anhydride thereof is condensed with an aryl amine of the formula III

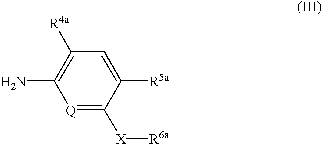

in which Q and X are as defined above and R^{4a}, R^{5a} and R^{6a} denote the radicals R⁴, R⁵ and R⁶ defined above or are substituents which can be converted by known processes (see, for example, the comments under B and C) into the radicals R⁴, R⁵ and R⁶.

The resulting cyclic imides of the formula II can then be converted by known processes into the 1-aryl-2-[1H]pyridones of the formula I. This reaction sequence is shown in an exemplary manner for the reaction of III with the 1,5-dicarboxylic acid IV (or its inner anhydride) in scheme 1:

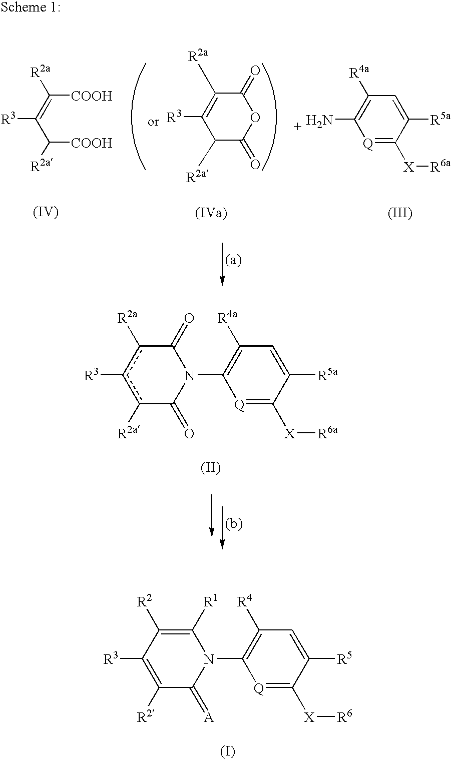

In scheme 1, the variables Q, A, X, R¹, R², R³, R⁴, R⁵, R⁶, R^{4a}, R^{5a} and R^{6a} are as defined above. R^{2a} and R^{2a′} have the meanings mentioned for R² and R^{2′}, respectively, which are different from amino, or denote substituents which can be converted by known processes (see, for example, the comments under B) into the radicals R² and R^{2′}, respectively. In formula II, ______ denotes in each case a double and a single bond. With respect to the presence and the position of the double bonds in IV or IVb, scheme 1 is not to be understood as imposing any limitations.

A.1a Step a

The condensation of aryl amines of the formula III with 1,5-dicarboxylic acids, preferably with dicarboxylic acids of the formula IV shown in scheme 1, or their anhydrides IVa, to the corresponding N-arylpiperidinediones or to the N-aryl-1H, 3H-dihydropyridine-2,6-diones of the formula II is carried out similarly to known processes for preparing such compounds, for example according to J. A. Seijas, J. Chem. Res. Synop. 1999, 7, 420-421; V. R. Ranade, J. Indian Chem. Soc. 1979, 56, 393-395; G. W. Joshi, Indian J. Chem. 1981, 20 B, 1050-1052; A. K. Ghosal, Indian J. Chem. 1978, 16B, 200-204. The complete disclosure of these publications is expressly incorporated herein by way of reference.

It is preferred to react a dicarboxylic acid IV or its double bond isomer with the aniline derivative of the formula III. The reaction is generally carried out by heating the components in an inert solvent or in the melt, preferably to temperatures above 100° C. and in particular to temperatures in the range from 120 to 300° C. (see also V. R. Ranade, loc. cit.).

Suitable solvents are aromatic and aliphatic hydrocarbons, such as toluene, xylene, isopropylbenzene, p-cumene, decalin and similar hydrocarbons, and also high-boiling ethers, for example dimethyl diethylene glycol and dimethyl triethylene glycol, and mixtures of the abovementioned solvents.

Instead of using elevated temperature, step a can also be effected by action of waves in the centimeter range (microwaves)(see J. A. Seijas, loc. cit.). Here, too, the reaction can be carried out in one of the abovementioned solvents or a diluent or in an intimate mixture of the components.

Preference is given to employing the components of the condensation step a, i.e. the 1,5-dicarboxylic acid IV or its anhydride IVa and the aryl amine III, in approximately equimolar amounts. It is, of course, also possible to use one of the components in excess.

Work-up of the reaction mixture of the condensation step a to prepare the compounds of the formula II is carried out by known processes, for example by crystallization, aqueous-extractive work-up or by chromatographic methods, or by combinations of these methods. It is, of course, also possible to use the compound II directly, without intermediate isolation or purification, in the next step.

The condensation step a shown in scheme 1 can take place in one step or else via intermediates, for example via acyclic amides, in particular if the anhydride IVa is used for condensation (compare G. W. Joshi, loc. cit., and also A. K. Gosal, loc. cit.). Any acyclic amides which may be formed can be cyclized both thermally, i.e. by reacting the amide in a high-boiling solvent or in the melt or in the presence of dehydrating agents such as acetic anhydride, oxalyl chloride or similar reagents and/or in the presence of a base such as piperidine, pyridine, dimethylaminopyridine or triethylamine.

The aryl amines of the formula III used in the condensation step are known, for example, from P. Böger and K. Wakabayashi, Peroxidizing Herbicides, Springer Verlag 1999, p. 21 ff. and literature cited therein, or they can be prepared by the methods described in WO 01/12625 or WO 97/08170.

The 1,5-dicarboxylic acids of the formula IV can be prepared by known methods for preparing 1,5-dicarboxylic acids. Particularly suitable for preparing the dicarboxylic acids IV is the synthesis sequence shown in scheme 2. The synthesis sequence shown in scheme 2 is similar to the process described by M. Guillaume, Synthesis 1995, 920-922.

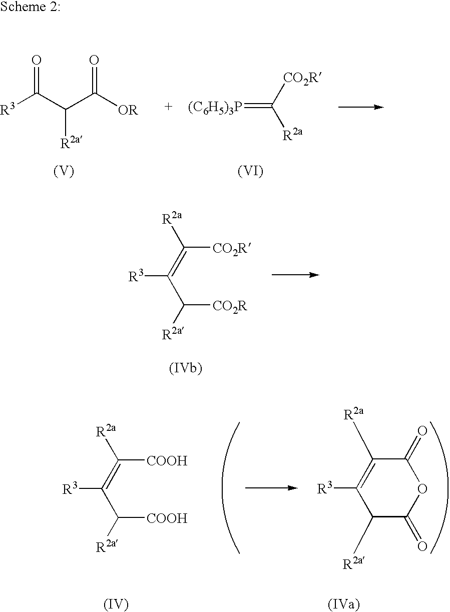

In scheme 2, R^{2a}, R^{2a′} and R³ are as defined above. R and R′ are radicals which can be hydrolyzed, preferably C₁-C₄-alkyl radicals, such as methyl or ethyl. With respect to the position of the double bonds in the compounds IV and IVa, scheme 2 is not to be understood as imposing any limitations.

The first step in scheme 2 is the reaction of a 2-haloacylalkanecarboxylic ester (for example a 2-haloacylacetic ester if R^{2a′}=H or a 2-haloacylpropionic ester if R^{2a′}=CH₃) of the formula V with a Wittig reagent, for example a phosphorylene of the formula VI. This gives the 3-haloalkyl-1,5-dicarboxylic esters of the formula IVb. This step is carried out under the reaction conditions which are customary for a Wittig reaction, as described, for example, in “Organikum”, 16. Edition, VEB Deutscher Verlag der Wissenschaften, Berlin 1986, p. 486, in M. Guillaume, Synthesis 1995, 920-922, and in the literature cited in J. March, Advanced Organic Chemistry, 2nd Edition, Wiley Interscience 1985, pp. 845-854, for Wittig reactions.

The subsequent hydrolysis of the dicarboxylic esters IVb to give the dicarboxylic acids IV is carried out by standard methods, for example by reacting IVb with alkali such as sodium hydroxide or potassium hydroxide in suitable solvents, for example in water, alcohols or in water/alcohol mixtures, at temperatures in the range from 0 to 200° C., preferably above 0° C., for example at boiling point or at room temperature.

The conversion of the dicarboxylic acids IV into their anhydrides IVa is likewise carried out by standard methods, for example by heating and/or in the presence of dehydrating agents such as acetic anhydride (G. W. Joshi, loc. cit.; A. Nangia, Synth. Commun. 1992, 22, 593-602) or in the presence of carbodiimides such as dicyclohexylcarbodiimide (compare N. M. Gray, J. Med. Chem. 1991, 34, 1283-1292). The publications mentioned for scheme 2 are expressly incorporated herein in their entirety by way of reference.

A.1b Step b

For converting the primary condensation product of the formula II obtained according to scheme 1 into compounds of the formula I in which R¹ is a halogen atom, the compound II is reacted with a halogenating agent, preferably an acidic halogenating agent, such as phosphorus trihalide, for example phosphorus trichloride, phosphorus(V) halide, for example phosphorus pentachloride, or phosphorus oxytrihalide, for example POCl₃, where preference is given to the last-mentioned halogenating agents, (see also M. S. Mayadeo, Indian J. Chem. 1987, 1099-1101 and Houben-Weyl, Methoden der Organischen Chemie [Methods of Organic Chemistry], Vol. 5/3, 1962, 4. Edition, pp. 899 ff. and 905 ff., which are included herein in their entirety by way of reference). This gives compounds of the formula I in which R1 is halogen, in particular chlorine.

The reaction with the halogenating agent can be carried out in an inert organic solvent, for example one of the abovementioned aromatic or aliphatic hydrocarbons and/or a halogenated hydrocarbon such as dichloromethane, dichloroethane, dichloroethene or trichloroethane, or using the halogenating agent as solvent. In general, the reaction is carried out with heating or under the action of waves in the centimeter range.

A.2 Nucleophilic Substitution

Compounds of the formula I where R¹=hydrogen can be prepared by reacting suitably substituted 2-[1H]-pyridones of the formula VII with nucleophilically substitutable aromatic compounds of the formula VIII, according to the synthesis sequence shown in scheme 3.

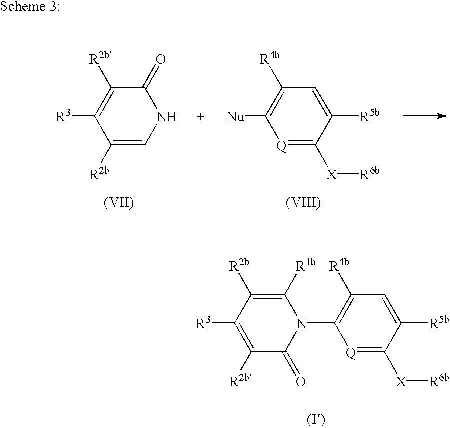

In scheme 3, the variables Q, X and R³ are as defined above. R^{1b}, R^{2b} and R^{2b′} are hydrogen or C₁-C₄-alkyl. R^{4b}, R^{5b} and R^{6b} have the meanings mentioned above for R⁴, R⁵ and R⁶, respectively, or denote substituents which can be converted by known processes into substituents R⁴, R⁵ and R⁶. Nu represents a nucleophilically displaceable leaving group, preferably a halogen atom, in particular chlorine and especially fluorine. In scheme 3, R^{5b} preferably represents an electron-withdrawing radical, in particular a cyano group or halogen. In the reaction of VII with VIII according to scheme 3, compounds of the formula I′ are obtained which can be used to prepare further compounds of the formula I by converting the groups R^{2b} to R^{6b} according to known methods, for example by the processes described under B) and C).

The reaction of VII with VIII to give the compounds I′ can be carried out, for example, similarly to the methods described in EP 259 048 or GB 8621217. This reaction is preferably carried out in the presence of a base, preferably an alkali metal hydride such as sodium hydride or an alkali metal carbonate such as sodium carbonate or potassium carbonate. If appropriate, copper or copper salts can be added as catalysts. If appropriate, it is also possible to add a crown ether as auxiliary catalyst.

The reaction is preferably carried out in a solvent, in particular a polar aprotic solvent such as dimethylformamide, dimethyl sulfoxide, N-methylpyrrolidone, dimethylacetamide, an ether such as diethyl ether, tetrahydrofuran or dioxane or mixtures of these solvents.

In general, the reaction is carried out at temperatures above room temperature, preferably in the range from 50 to 200° C. To this end, the compounds of the formulae VII and VIII are preferably employed in approximately equimolar amounts. It is, of course, also possible to use one component in excess, the excess preferably not being more than 50 mol %, in particular not more than 20 mol %, based on the-component present in substoichiometric amounts.

Pyridones of the formula VII are known, some of them are commercially available, or they can be prepared similarly to known processes for preparing pyridones. Pyridones of the formula VII can be prepared, for example, from suitably substituted 2-chloropyridines. To this end, the 2-chloropyridine is successively converted into its benzyl ether (compare A. J. S. Duggan et al., Synthesis 1980, 7, 573 and A. Loupy et al., Heterocycles 1991, 32, 1947-1953; these publications are included herein by way of reference) and subsequent hydrogenolysis by the method described in T. W. Greene, Protective Groups in Organic Synthesis, 3. Edition 1999, p. 266ff.

Compounds of the formula VIII are commercially available or can be prepared by known methods, for example by Sandmeyer reaction from the corresponding anilines II (cf. Böger et al. in Peroxidizing Herbicides).

Following the preparation of I′, it is, of course, also possible to convert the substituents R^{1b} to R^{6b} contained therein into other substituents R¹ to R⁶. Processes to achieve this are known and described, for example, in sections B) and C) below.

### B) Functionalization of the Substituents on the Pyridone Moiety of I

Compounds of the formula I in which A is an oxygen atom can be converted according to known methods by treatment with sulfurizing agents into compounds of the formula I in which A is a sulfur atom. Examples of suitable sulfurizing agents are phosphorus(V) sulfide, organotin sulfides and organophosphorus sulfides (see also J. March, Advanced Organic Synthesis, 2nd Edition, Wiley Interscience 1985, p. 794 and literature cited therein). The reaction can be carried out in a solvent or neat. Suitable solvents are the abovementioned inert solvents and basic solvents, for example pyridine and the like. The temperature required for the reaction is generally above room temperature and in particular in the range from 50 to 200° C.

Compounds of the formula I in which R² or R^{2′} are hydrogen can also be converted by known processes for functionalizing pyridones into compounds in which R² or R^{2′} represent an amino group.

Compounds I in which one or both of the radicals R² and R^{2′} are amino are prepared by successive nitration and hydrogenation, similarly to the procedure of DE-A 20 55 513.

### C) Compounds I Where Q=CH (Compounds IA) can be Converted by Functionalization of the Phenyl Ring Into Other Compounds IA

Examples are: ps C.1 Nitration of 1-Arylpyridones IA in Which XR⁶ is Hydrogen and Conversion of the Process Products Into Further Compounds of the Formula IA:

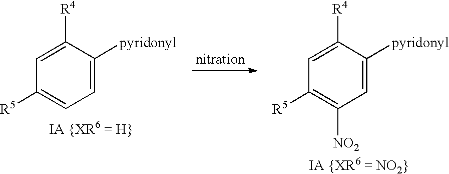

Suitable nitrating agents are, for example, nitric acids in varying concentration, including concentrating and fuming nitric acid, mixtures of sulfuric acid and nitric acid, and furthermore acetyl nitrates and alkyl nitrates.

The reaction can either be carried out in the absence of a solvent using an excess of nitrating agent or in an inert solvent or diluent, suitable solvents or diluents being, for example, water, mineral acids, organic acids, halogenated hydrocarbons such as methylene chloride, anhydrides such as acetic anhydride and mixtures of these solvents.

Starting material IA {XR⁶=H} and nitrating agent are advantageously employed in approximately equimolar amounts; however, to optimize the conversion of starting material, it may be advantageous to employ an excess of nitrating agent, up to about 10 times the molar amount, based on IA. If the reaction is carried out in the absence of a solvent in the nitrating agent, the latter is present in an even greater excess.

The reaction temperature is usually from −100° C. to 200° C., preferably from −30 to 50° C.

The compounds IA where XR⁶=NO₂ can then be reduced to compounds IA where X—R⁶=NH₂ or —NHOH:

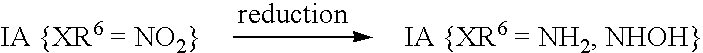

In general, the reduction is carried out by reacting the nitro compound with a metal such as iron, zinc or tin under acidic reaction conditions or using a complex hydride such as lithium aluminum hydride or sodium borohydride, it being possible to carry out the reduction neat or in a solvent or diluent. Suitable solvents are—depending on the selected reducing agent—for example water, alcohols such as methanol, ethanol and isopropanol or ethers such as diethyl ether, methyl tert-butyl ether, dioxane, tetrahydrofuran and ethylene glycol dimethyl ether.

If the reduction is carried out using a metal, the reaction is preferably carried out in the absence of a solvent using an inorganic acid, in particular in concentrated or dilute hydrochloric acid, or in a liquid organic acid such as acetic acid or propionic acid. However, it is also possible to dilute the acid with an inert solvent, for example one of those mentioned above. The reduction with complex hydrides is preferably carried out in a solvent, for example in ether or in alcohol.

The nitro compound IA {X—R⁶=NO₂} and the reducing agent are frequently employed in approximately equimolar amounts; to optimize the course of the reaction, it may be advantageous to use an excess of one of the two components, up to about 10 times the molar amount.

The amount of acid is not critical. To achieve as complete a reduction of the starting material as possible, it is advantageous to use at least an equivalent amount of acid. Frequently, the acid is employed in excess based on IA {X—R⁶=NO₂}.

The reaction temperature is generally in the range from −30° C. to 200° C., preferably in the range from 0° C. to 80° C.

For work-up, the reaction mixture is generally diluted with water and the product is isolated by filtration, crystallization or extraction with a solvent which is substantially water-immiscible, for example with ethyl acetate, diethyl ether or methylene chloride. If desired, the product can then be purified as usual.

It is also possible to hydrogenate the nitro group of the compounds IA {X—R⁶=NO₂} catalytically using hydrogen. Catalysts which are suitable for this purpose are, for example, Raney nickel, palladium-on-carbon, palladium oxide, platinum and platinum oxide, an amount of catalyst of from 0.05 to 10.0 mol %, based on the compound to be reduced, generally being sufficient.

The reaction is carried out either in the absence of a solvent or in an inert solvent or diluent, for example in acetic acid, a mixture of acetic acid and water, ethyl acetate, ethanol or in toluene.

Following removal of the catalyst, the reaction solution can be worked up as usual to afford the product.

The hydrogenation can be carried out at atmospheric hydrogen pressure or under elevated hydrogen pressure.

The amino group in IA {X—R⁶=NH₂} can then be diazotized in a customary manner. From the diazonium salts, compounds I are then obtainable in which:

X—R⁶=cyano or halogen {for example by Sandmeyer reaction: cf., for example, Houben-Weyl, Methoden der Organischen Chemie, Georg Thieme Verlag Stuttgart, Vol. 5/4, 4. Edition 1960, p. 438ff.},

X—R⁶=hydroxyl {for example by heating the diazonium salt to give the phenol: cf., for example, Org. Synth. Coll. Vol. 3 (1955), p. 130},

X—R⁶=mercapto or C₁-C₆-alkylthio {cf., for example, Houben-Weyl, Methoden der Organischen Chemie, Georg Thieme Verlag Stuttgart, Vol. E11 1984, p. 43 and 176},

X—R⁶=halosulfonyl {cf., for example, Houben-Weyl, Methoden der Organischen Chemie, Georg Thieme Verlag Stuttgart, Vol. E11 1984, p. 1069f.},

X—R⁶=for example —CH₂—CH(halogen)—CO—O—Y—R⁸, —CH═C(halogen)—CO—O—Y—R⁸, —CH₂—CH(halogen)—PO—(O—Y—R⁸)₂, —CH═C(halogen)—PO—(O—Y—R⁸)₂ {in general, these are products of a Meerwein arylation; cf., for example, C. S. Rondestredt, Org. React. 11, 189 (1960) and H. P. Doyle et al., J. Org. Chem. 42, 2431 (1977)}.

The diazonium salt of IA {X—R⁶=N₂⁺} is in each case generally prepared in a manner known per se by reacting IA {X—R⁶=NH₂} with a nitrozating agent, for example a nitrite such as sodium nitrite or potassium nitrite in an aqueous solution of an acid, for example in hydrochloric acid, hydrobromic acid or sulfuric acid.

To prepare the diazonium salt IA {X—R⁶=N₂⁺}, the amino compound IA {X—R⁶=NH₂} can be reacted with a nitrous acid ester such as tert-butyl nitrite or isopentyl nitrite under anhydrous reaction conditions, for example in hydrogen chloride-containing glacial acetic acid, in absolute alcohol, in dioxane or tetrahydrofuran, in acetonitrile or in acetone.

Conversion of the resulting diazonium salt into the corresponding compound IA where X—R⁶=cyano, chlorine, bromine or iodine is particularly preferably carried out by treatment with a solution or suspension of a copper(I) salt such as copper(I) cyanide, chloride, bromide or iodide, or with an alkali metal salt solution (cf. A1).

The resulting diazonium salt is advantageously converted into the corresponding hydroxyl compound IA {X—R⁶=hydroxyl} by treating the diazonium salt IA with an aqueous acid, preferably sulfuric acid. Addition of a copper(II) salt such as copper(II) sulfate may be advantageous for the course of the reaction. In general, this reaction is carried out at from 0 to 100° C., preferably at the boiling point of the reaction mixture.

Compounds IA where X—R⁶=mercapto, C₁-C₆-alkylthio or halosulfonyl are obtained, for example, by reacting the corresponding diazonium salt of IA with hydrogen sulfide, an alkali metal sulfide, a dialkyl disulfide such as dimethyl disulfide or with sulfur dioxide.

The Meerwein arylation is usually the reaction of the diazonium salts with alkenes or alkynes. The alkene or alkyne is advantageously employed in excess, up to about 3000 mol %, based on the amount of diazonium salt.

The reactions described above of the diazonium salt IA {X—R⁶=N₂⁺} can be carried out, for example, in water, in aqueous hydrochloric acid or hydrobromic acid, in a ketone such as acetone, diethyl ketone or methyl ethyl ketone, in a nitrile such as acetonitrile, in an ether such as dioxane or tetrahydrofuran or in an alcohol such as methanol or ethanol.

Unless mentioned otherwise for the specific reactions, the reaction temperatures are usually from −30° C. to 50° C.

All reaction partners are preferably employed in approximately stoichiometric amounts; however, an excess of one component or the other of up to about 3000 mol % may be advantageous.

The mercapto compounds IA {X—R⁶=SH} can also be obtained by reducing the compounds IA described below in which X—R⁶=halosulfonyl. Useful reducing agents are, for example, transition metals such as iron, zinc and tin (cf., for example, “The Chemistry of the Thiol Group”, John Wiley, 1974, p. 216).

C.2 Halosulfonation of 1-Arylpyridones IA, in Which XR⁶ is Hydrogen:

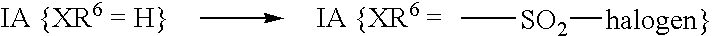

The halosulfonation can be carried out in the absence of a solvent in an excess of sulfonating agent or in an inert solvent/diluent, for example in a halogenated hydrocarbon, in ether, in alkylnitrile or a mineral acid.

Chlorosulfonic acid is both the preferred reagent and the preferred solvent.

The sulfonating agent is usually employed in a slightly substoichiometric amount (up to about 95 mol %) or in an excess of 1 to 5 times the molar amount, based on the starting material IA (where X—R⁶=H). If the reaction is carried out in the absence of inert solvent, it may be advantageous to use an even greater excess.

The reaction temperature is usually between 0° C. and the boiling point of the reaction mixture.

For work-up, the reaction mixture is mixed, for example, with water, and the product can then be isolated as usual.

C.3 Side Chain Halogenation of 1-Arylpyridones IA in Which X—R⁶ is Methyl and Conversion of the Process Products Into Further Compounds of the Formula IA:

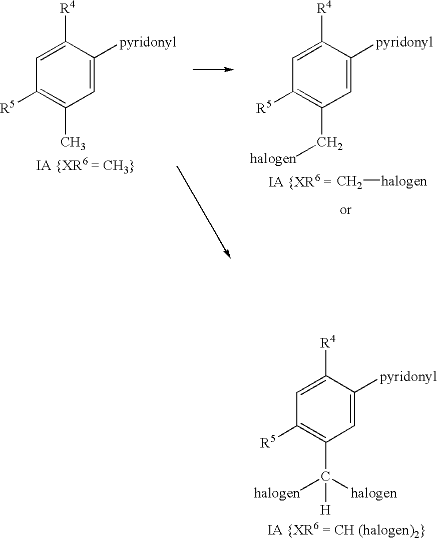

Examples of suitable solvents are organic acids, inorganic acids, aliphatic or aromatic hydrocarbons, which may be halogenated, and also ethers, sulfides, sulfoxides and sulfones.

Suitable halogenating agents are, for example, chlorine, bromine, N-bromosuccinimide, N-chlorosuccinimide or sulfuryl chloride. Depending on the starting material and the halogenating agent, addition of a free-radical initiator, for example an organic peroxide such as dibenzoyl peroxide, or an azo compound such as azobisisobutyronitrile, or irradiation with light may be advantageous for the course of the reaction.

The amount of halogenating agent is not critical. It is possible to use either substoichiometric amounts or large excesses of halogenating agent, based on the compound IA to be halogenated (where X—R⁶=methyl).

If a free-radical initiator is used, a catalytic amount thereof is usually sufficient.

The reaction temperature is usually from −100° C. to 200° C., preferably from 10 to 100° C. or the boiling point of the reaction mixture.

The halogenation products IA where X—R⁶=CH₂-halogen can be converted in a nucleophilic substitution reaction according to the scheme below into the corresponding ethers, thioethers, esters, amines or hydroxylamines:

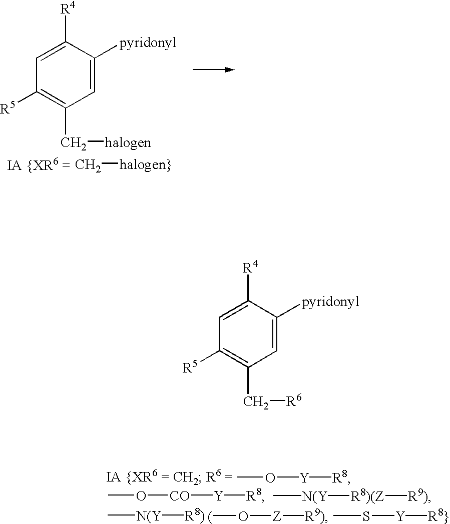

Suitable for use as nucleophiles are either the corresponding alcohols, thiols, carboxylic acids or amines, in which case the reaction is preferably carried out in the presence of a base (for example in alkali metal hydroxide or alkaline earth metal hydroxide or alkali metal carbonate or alkaline earth metal carbonate), or the alkali metal salts of these compounds obtained by reaction of a base (for example an alkali metal hydride) with the alcohols, thiols, carboxylic acids or amines are used.

Suitable solvents are in particular aprotic organic solvents, for example tetrahydrofuran, dimethylformamide, dimethyl sulfoxide, or hydrocarbons such as toluene and n-hexane.

The reaction is carried out at a temperature between the melting point and the boiling point of the reaction mixture, preferably at from 0 to 100° C.

The halogenation products IA where X—R⁶=CH(halogen)₂ can be hydrolyzed to give the corresponding aldehydes (IA where X—R⁶=CHO). The latter in turn can be oxidized similarly to known processes to give the carboxylic acids {X—R⁶=COOH}:

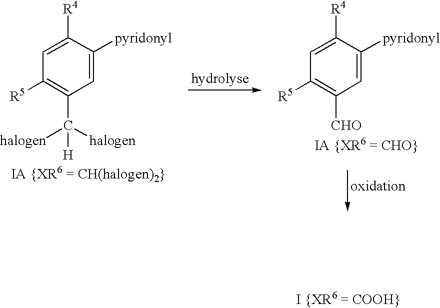

The hydrolysis of the compounds IA where X—R⁶=dihalomethyl is preferably carried out under acidic conditions, in particular in the absence of a solvent in hydrochloric acid, acetic acid, formic acid or sulfuric acid, or else in an aqueous solution of one of the acids mentioned, for example in a mixture of acetic acid and water (for example 3:1).

The reaction temperature is usually from 0 to 120° C.

The oxidation of the hydrolysis products IA where XR⁶=formyl to give the corresponding carboxylic acids can be carried out in a manner known per se, for example according to Kornblum (see, in particular, pages 179 to 181 of the volume “Methods for the Oxidation of Organic Compounds” by A. H. Haines, Academic Press 1988, in the series “Best Synthetic Methods”). A suitable solvent is, for example, dimethyl sulfoxide.

The aldehydes IA {X—R⁶=CHO} can also be olefinated in a manner known per se to give compounds IA where X=unsubstituted or substituted ethene-1,2-diyl:

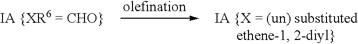

The olefination is preferably carried out by the method of Wittig or one of its modifications, suitable reaction partners being phosphorylides, phosphonium salts and phosphonates, or by aldol condensation.

If a phosphonium salt or a phosphonate is used, it is recommended to carry out the reaction in the presence of a base, particularly suitable bases being alkali metal alkyls such as n-butyllithium, alkali metal hydrides and alkoxides such as sodium hydride, sodium ethoxide and potassium tert-butoxide, and also alkali metal hydroxides and alkaline earth metal hydroxides such as calcium hydroxide.

To achieve complete conversion, all reaction partners are employed in approximately stoichiometric ratios; however, preference is given to using an excess of phosphorus compound and/or base of up to about 10 mol %, based on the starting material (IA where X—R⁶=CHO).

The reaction temperature is generally from −40 to 150° C.

The 1-arylpyridones IA where X—R⁶=formyl can be converted in a manner known per se into compounds IA where X—R⁶=—CO—Y—R⁸, for example by reaction with a suitable organometallic compound Me—Y—R⁸—where Me is a base metal, preferably lithium or magnesium—and subsequent oxidation of the resulting alcohols (cf., for example, J. March, Advanced Organic Chemistry, 3rd ed., John Wiley, New York 1985, p. 816ff. and 1057ff.).

For their part, the compounds IA where X—R⁶=—CO—Y—R⁸ can be converted further in a reaction according to Wittig. The phosphonium salts, phosphonates or phosphorylides required as reaction partners for this purpose are known or can be prepared in a manner known per se {cf., for example, Houben-Weyl, Methoden der Organischen Chemie, Vol. E1, p. 636ff. and Vol. E2, p. 345ff., Georg Thieme Verlag Stuttgart 1982; Chem. Ber. 95, 3993 (1962)}.

Further possibilities for preparing other 1-arylpyridones IA from compounds IA where X—R⁶=formyl include the aldol condensation, which is known per se, and also condensation reactions according to Knoevenagel or Perkin. Suitable conditions for these processes can be found, for example, in Nielson, Org. React. 16, lff (1968) {aldol condensationr} Org. React. 15, 204ff. (1967) {condensation according to Knoevenagel} and Johnson, Org. React. 1, 210ff. (1942) {condensation according to Perkin}.

It is also possible to convert the compounds IA where X—R⁶=—CO—Y—R⁸ in a manner known per se into the corresponding oximes {cf., for example, Houben-Weyl, Methoden der Organischen Chemie, Georg Thieme Verlag Stuttgart, Vol. 10/4, 4. Edition 1968, p. 55ff. and p. 73ff.}:

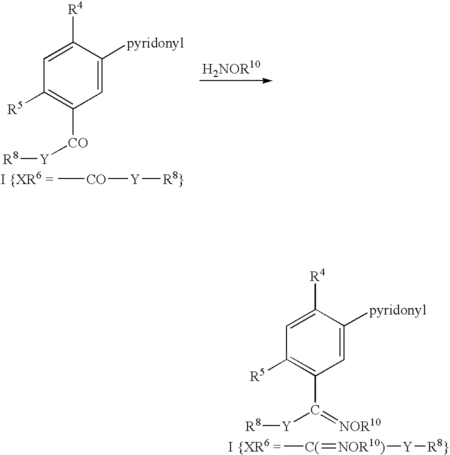

C.4 Synthesis of Ethers, Thioethers, Amines, Esters, Amides, Sulfonamides, Thioesters, Hydroximic Acid Esters, Hydroxylamines, Sulfonic Acid Derivatives, Oximes or Carboxylic Acid Derivatives:

1-Arylpyridones IA in which R⁶ is hydroxyl, amino, —NH—Y—R⁸, hydroxylamino, —N(Y—R⁸)—OH, —NH—O—Y—R⁸, mercapto, halosulfonyl, —C(═NOH)—Y—R⁸, carboxyl or —CO—NH—O—Z—R⁹ can be converted in a manner known per se by alkylation, acylation, sulfonylation, esterification or amidation into the corresponding ethers {IA where R⁶=—O—Y—R⁸}, esters {I where R⁶=—O—CO—Y—R⁸}, amines {I where R⁶=—N(Y—R⁸)(Z—R⁹)}, amides {IA where R⁶=—N(Y—R⁸)—CO—Z—R⁹}, sulfonamides {IA where R⁶=—N(Y—R⁸)—SO₂—Z—R⁹ or —N(SO₂—Y—R⁸)(SO₂—Z—R⁹)}, hydroxylamines {IA where R⁶=—N(Y—R⁸)(O—Z—R⁹)}, thioethers {IA where R⁶=—S—Y—R⁸}, sulfonic acid derivatives {IA where R⁶=—SO₂—Y—R⁸, —SO₂—O—Y—R⁸ or —SO₂—N(Y—R⁸)(Z—R⁹)}, oximes (IA where R⁶=—C(═NOR¹⁰)—Y—R⁸}, carboxylic acid derivatives {IA where R⁶=—CO—O—Y—R⁸, —CO—S—Y—R⁸, —CO—N(Y—R⁸)(Z—R⁹), —CO—N(Y—R⁸)(O—Z—R⁹)} or hydroximic acid esters {I where R⁶=—C(═NOR¹⁰)—O—Y—R⁸}.

Such reactions are described, for example, in Houben-Weyl, Methoden der Organischen Chemie, Georg Thieme Verlag Stuttgart (Vol. E16d, p. 1241ff.; Vol. 6/1a, 4. Edition 1980, p. 262ff.; Vol. 8, 4. Edition 1952, p. 471ff., 516ff., 655ff. and p. 686ff.; Vol. 6/3, 4. Edition 1965, p. 10ff.; Vol. 9, 4. Edition 1955, p. 103ff., 227ff., 343ff., 530ff., 659ff., 745ff. and p. 753ff.; Vol. E5, p. 934ff., 941ff. and p. 1148ff.).

Ethers (compounds I where X—R⁶=O—Y—R⁸), for example, can be prepared in good yields by reacting the corresponding hydroxyl compound (compound I where X—R⁶=OH) with an aliphatic halide Hal-Y—R⁸ (Hal=chlorine, bromine or iodine). The reaction is carried out in the manner described for the alkylation of phenols (for the ether synthesis, see, for example, J. March “Advanced Organic Chemistry” 3rd ed. p. 342 f. and literature cited therein), preferably in the presence of a base such as NaOH or an alkali metal carbonate or sodium hydride. Preferred reaction media are aprotic polar solvents such as dimethylformamide, N-methylpyrrolidone or dimethylacetonitrile.

C.5 Nucleophilic Substitution of Compound I in Which X—R⁶ is Halogen.

The scheme below shows examples of the classes of compounds obtainable by this route.

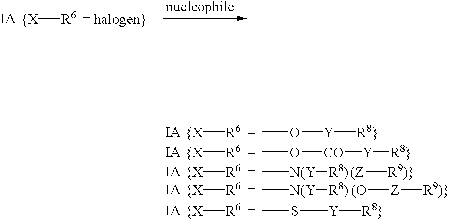

Suitable nucleophiles are alcohols, thiols, amines, carboxylic acids or CH-acidic compounds, for example nitroalkanes such as nitromethane, malonic acid derivatives such as diethyl malonate or cyanoacetic acid derivatives, such as methyl cyanoacetate.

This reaction has particularly good results in the case of the compounds IA in which R⁵ is an electron-withdrawing radical, for example a trifluoromethyl group or a cyano group.

The reaction is preferably carried out in the presence of a strong base, for example one of the bases mentioned for A2. It is, of course, also possible to deprotionate the abovementioned nucleophiles quantitatively prior to the reaction, using a strong base. With respect to the reaction conditions, reference is made to what has been said under A.2. Furthermore, reference is made to J. March, Advanced Organic Synthesis, 3. Edition 1985, p. 576 and the literature cited therein.

### D) Preparation of Compounds of the Formula I in Which Q is a Nitrogen Atom (Compounds IB)

In addition to the processes already mentioned in the preceding sections A, B and C, processes D.1 and D.2 below are particularly suitable for this purpose:

D.1 Halogenation of the Pyridine Ring of Compounds IB Where X—R⁶=H: to this end, Preference is Given to Initially Converting a 3-Pyridylpyridone of the Formula IB (X—R⁶=H) Into the Corresponding Pyridine N-Oxide of the Formula IX.

In the formula IX, R¹, R², R⁴ and R⁵ are as defined above.

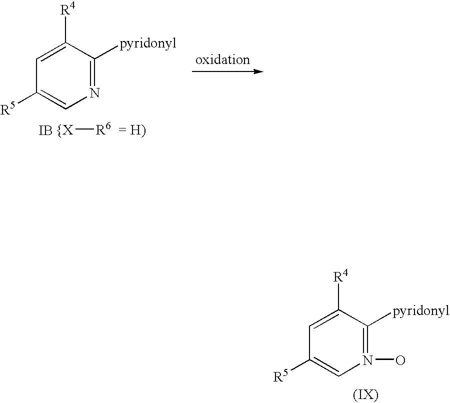

Oxidizing agents which are suitable for this reaction are, for example, hydrogen peroxide or organic peracids, for example performic acid, peracetic acid, trifluoroperacetic acid or m-chloroperbenzoic acid.

Suitable solvents are organic solvents which are inert to oxidation, such as, for example, hydrocarbons such as toluene or hexane, ethers such as diethyl ether, dimethoxyethane, methyl tert-butyl ether, dioxane or tetrahydrofuran, alcohols such as methanol or ethanol, or else mixtures of such solvents with one another or with water. If the oxidation is carried out using an organic peracid, the preferred solvent is the parent organic acid, i.e., for example, formic, acetic or trifluoroacetic acid, if appropriate in a mixture with one or more of the abovementioned solvents.

The reaction temperature is usually between the melting point and the boiling point of the reaction mixture, preferably at 0-150° C.

To obtain a high yield, it is frequently advantageous to employ the oxidizing agent in a molar excess of up to about five times, based on the IB (where X—R⁶=H) used.

The pyridine N-oxide IX is then converted by reaction with a halogenating agent into IB (X—R⁶=halogen).

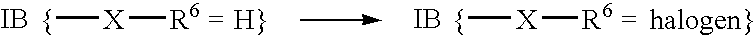

Suitable halogenating agents are phosphoryl halides such as POCl₃ or POBr₃, phosphorus halides such as PCl₅, PBr₅, PCl₃ or PBr₃, phosgene or organic or inorganic acid halides such as, for example, trifluoromethanesulfonyl chloride, acetyl chloride, bromoacetyl bromide, acetyl bromide, benzoyl chloride, benzoyl bromide, phthaloyl dichloride, toluenesulfonyl chloride, thionyl chloride or sulfuryl chloride. If appropriate, it may be advantageous to carry out the reaction in the presence of a base, such as, for example, trimethylamine or triethylamine or hexamethyldisilazane.

Suitable solvents are inert organic solvents, such as, for example, hydrocarbons such as toluene or hexane, ethers such as diethyl ether, dimethoxyethane, methyl tert-butyl ether, dioxane or tetrahydrofuran, amides such as DMF, DMA or NMP, or mixtures thereof. If the reaction is carried out using a liquid halogenating agent, this may preferably also be used as solvent, if appropriate in a mixture with one of the abovementioned solvents.

The reaction temperature is usually between the melting point and the boiling point of the reaction mixture, preferably at 50-150° C.

To obtain a high yield, it may be advantageous to employ the halogenating agent or the base in an excess of up to about five times the molar amount, based on the IX used.

D.2 Nucleophilic Substitution on Halopyridines of the Formula IB (X—R⁶=Halogen).

The scheme below shows examples of the classes of compounds obtainable by this route.

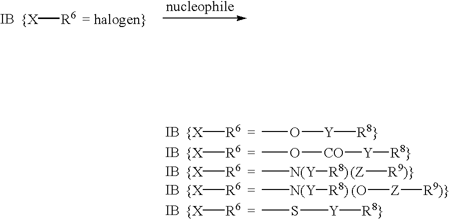

Suitable nucleophiles are alcohols, thiols, amines, carboxylic acids or CH acidic compounds, for example nitroalkanes such as nitromethane, malonic acid derivatives such as diethyl malonate or cyanoacetic acid derivatives, such as methyl cyanoacetate. For the practice of this reaction, what has been said under C.5 applies.

### E) Preparation of Compounds of the Formula I in Which R⁷ Together with X—R⁶ Denotes One of the Chains —N═C(R¹⁹)—S— (Compounds IC-1) or —N═C(R¹⁹)—O— (Compounds IC-2).

To prepare the compounds IC, it is also possible to employ the processes mentioned in sections A and B, or to use these processes for preparing suitable starting materials.

Furthermore, the compounds IC-1 and IC-2 can be synthesized similarly to known processes by ring-closure reaction from the corresponding ortho-aminophenols or ortho-mercaptoanilines of the formulae IA-1 and IA-2; on this subject, numerous methods are disclosed in the literature (see, for example, Houben-Weyl, Methoden der Organischen Chemie, Vol. E8a, p.1028ff., Georg-Thieme-Verlag, Stuttgart 1993 and Vol. E8b, p. 881ff., Georg-Thieme-Verlag, Stuttgart 1994). In the formulae IA-1 and IA-2, the variables “pyridonyl”, R⁴ and R⁵ are as defined above or denote substituents which can be converted into these groups by known methods. The variables X¹ and X² independently of one another denote OH or SH.

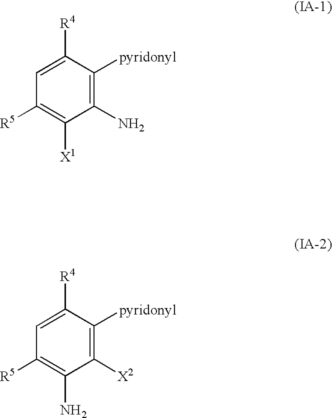

E.1 Compounds IC-1 in Which R⁷ Together With X—R⁶ Forms One of the Chains —N═C(R¹⁹)—S— can Also be Prepared, in Particular, by the Process Shown Below:

This process includes the reaction of an aminophenylpyridone of the formula IA-3 or IA-4 with halogen and ammonium thiocyanate or with an alkali metal thiocyanate or alkaline earth metal thiocyanate. This gives compounds of the formula IC-1a and IC-1b, respectively, where R¹⁹=NH₂.

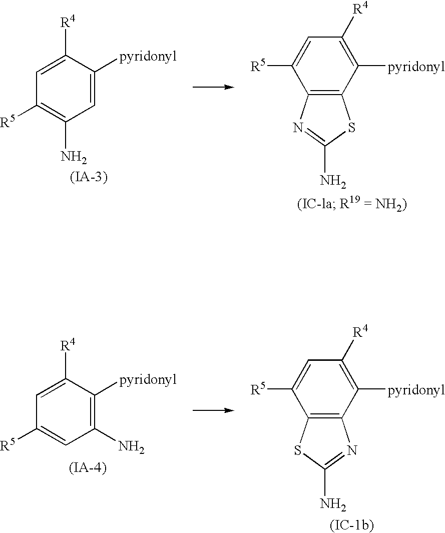

These compounds can be converted by subsequent reactions on the amino group into other compounds IC-1a or IC-1b.

Preferred halogen is chlorine or bromine; among the alkali/alkaline earth metal thiocyanates, sodium thiocyanate is preferred.

In general, the reaction is carried out in an inert solvent/diluent, for example in a hydrocarbon such as toluene and hexane, in a halogenated hydrocarbon such as dichloromethane, in an ether such as tetrahydrofuran, in an alcohol such as ethanol, in a carboxylic acid such as acetic acid, or in a polar aprotic solvent/diluent such as dimethylformamide, acetonitrile or dimethyl sulfoxide.

The reaction temperature is usually between the melting point and the boiling point of the reaction mixture, preferably at from 0 to 150° C.

To obtain a high yield of the product of value, halogen and ammonium thiocyanate or alkali/alkaline earth metal thiocyanate are preferably employed in approximately equimolar amount or in an excess, up to about 5 times the molar amount, based on the amount of IA-3 or IA-4.

One variant of the process comprises initially converting the NH₂ group of the aminophenyl pyridones IA-3 or IA-4 with ammonium thiocyanate or an alkali metal thiocyanate or alkaline earth metal thiocyanate into a thiourea group (NH—C(S)—NH₂ group) and then converting these compounds by treatment with a halogen into the benzothiazoles (compounds IC-1a or ID-1 where R¹⁹=NH₂).

Finally, reactions similar to those already described in section C.1) can be carried out on the amino group of the chain —N═C(NH₂)—S—, in order to introduce in this manner other radicals R¹⁹ into the compounds I.

E.2 Compounds of the Formula IC in Which R⁷ Together With X—R⁶ Forms One of the Chains —N═C(R¹⁹)—O— can be Prepared by Successive Conversion of the NH₂ Group in the Aminophenylpyridones of the Formula IA-3 or IA-4 Into an Azide Group (N₃ Group) and Subsequent Cyclization of the Resulting Azidophenylpyridones With a Carboxylic Acid to Give Compounds of the Formula IC-2a or IC-2b.

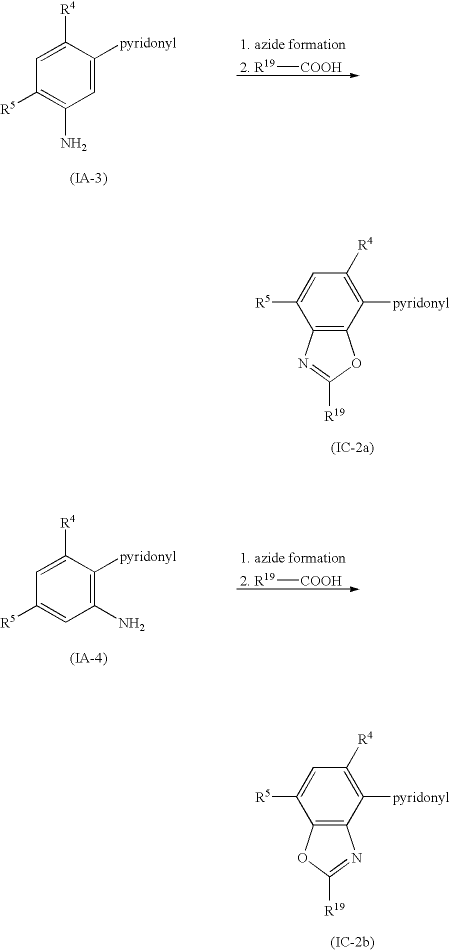

The conversion of the amino group in the aminophenylpyridones of the formula IA-3 or IA-4 into an azide group is generally carried out in two steps, i.e. by diazotizing the amino group and subsequent treatment of the resulting diazonium salt with an azide. For the practice of the diazotization, what has been said for process C.1) applies. The conversion into the arylazides is preferably carried out by reaction of diazonium salts with an alkali metal azide or alkaline earth metal azide such as sodium azide or by reaction with trimethylsilyl azide.

The reaction of the azide compounds IA (X—R⁶=N₃) with the carboxylic acid R¹⁹—COOH is either carried out in an inert organic solvent, for example in hydrocarbons such as toluene or hexane, in halogenated hydrocarbons such as dichloromethane or chloroform, in ethers such as diethyl ether, dimethoxyethane, methyl tert-butyl ether, dioxane or tetrahydrofuran, in amides such as dimethylformamide (DMF), dimethylacetamide (DMA) or N-methylpyrrolidone (NMP), in acetonitrile or preferably in the absence of a solvent in an excess of the carboxylic acid R¹⁹COOH. In the latter case, it may be helpful to add a mineral acid such as phosphoric acid or a silylating reagent such as a mixture of phosphorus pentoxide and hexamethyldisiloxane.

The reaction is preferably carried out at elevated temperature, for example at the boiling point of the mixture.

### F) The Compounds of the Formula I in Which X—R⁶ Together With R⁷ Forms One of the Chains —O—C(R¹⁶,R¹⁷)—CO—N(R¹⁸)— or —S—C(R¹⁶,R¹⁷)—CO—N(R¹⁸)— can be Prepared by the Processes Mentioned in Sections A and B

Moreover, in principle, they can be prepared from the corresponding aminophenols or mercaptoanilines IA-1 or IA-2 using known processes, for example the process described in U.S. Pat. No. 4,798,620. With respect to this reaction, the disclosure of this publication is expressly incorporated herein by way of reference.

In particular those compounds of the formula I in which X—R⁶ together with R⁷ forms a chain —O—C(R¹⁶,R¹⁷)—CO—N(R¹⁸)— can also be prepared from the nitrophenoxyacetic acid derivatives of the formulae IA-5 and IA-6. The conversion is carried out by reducing the nitro groups in IA-5 or IA-6 where generally simultaneously with the reduction a ring-closure reaction occurs, giving the compounds of the formula IC-3a or IC-3b.

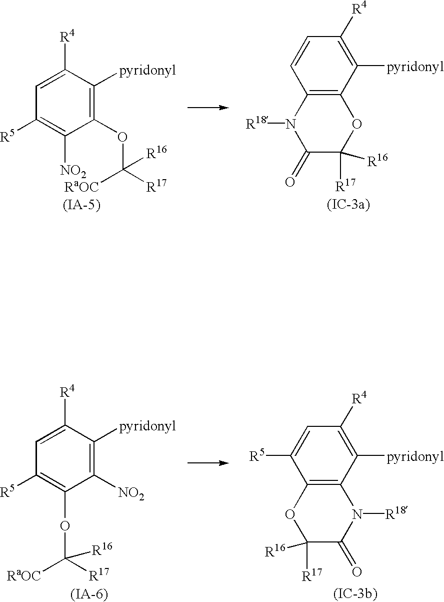

In the formulae IA-5, IA-6, IC-3a and IC-3b, “pyridonyl”, R⁴, R⁵, R¹⁶ and R¹⁷ are as defined above. R^{18′} is H or OH. Rₐ is a nucleophilically displaceable leaving group, for example a C₁-C₄-alkoxy radical such as methoxy or ethoxy.

These reductions can be carried out according to the conditions mentioned in section C.1) for the reduction of aromatic nitro groups.

If desired, the reaction products can be converted by alkylation into further compounds of the formula IC-3. For the practice of these reactions, what has been said in section C.4 applies correspondingly.

Unless stated otherwise, all the processes described above are advantageously carried out at atmospheric pressure or under the autogenous pressure of the reaction mixture in question.

The work-up of the reaction mixtures is usually carried out in a conventional manner. Unless stated otherwise in the processes described above, the products of value are obtained, for example, after the dilution of the reaction solution with water by filtration, crystallization or solvent extraction, or by removing the solvent, partitioning the residue in a mixture of water and a suitable organic solvent and work-up of the organic phase to afford the product.

The 1-arylpyridones of the formula I can be obtained as isomer mixtures in the preparation; however, if desired, these can be separated into largely pure isomers using customary methods such as crystallization or chromatography, including chromatography over an optically active adsorbent. Pure optically active isomers can be prepared advantageously from corresponding optically active starting materials.

Agriculturally useful salts of the compounds I can be formed by reaction with a base of the corresponding cation, preferably an alkali metal hydroxide or hydride, or by reaction with an acid of the corresponding anion, preferably hydrochloric acid, hydrobromic acid, sulfuric acid, phosphoric acid or nitric acid.

Salts of I where the metal ion is not an alkali metal ion can be prepared by cation exchange of the corresponding alkali metal salt in a conventional manner, similarly ammonium, phosphonium, sulfonium and sulfoxonium salts by means of ammonia, phosphonium, sulfonium or sulfoxonium hydroxides.

The compounds I and their agriculturally useful salts are suitable, both in the form of isomer mixtures and in the form of the pure isomers, for use as herbicides. The herbicidal compositions comprising compounds I or their salts control vegetation on non-crop areas very efficiently, especially at high rates of application. They act against broad-leaved weeds and grass weeds in crops such as wheat, rice, maize, soya and cotton without causing any significant damage to the crop plants. This effect is mainly observed at low rates of application.

Depending on the application method used, the compounds I or compositions comprising them, can additionally be employed in a further number of crop plants for eliminating undesirable plants. Examples of suitable crops are the following:

Allium cepa, Ananas comosus, Arachis hypogaea, Asparagus officinalis, Beta vulgaris spec. altissima, Beta vulgaris spec. rapa, Brassica napus var. napus, Brassica napus var. napobrassica, Brassica rapa var. silvestris, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N.rustica), Olea europaea, Oryza sativa , Phaseolus lunatus, Phaseolus vulgaris, Picea abies, Pinus spec., Pisum sativum, Prunus avium, Prunus persica, Pyrus communis, Ribes sylvestre, Ricinus communis, Saccharum officinarum, Secale cereale, Solanum tuberosum, Sorghum bicolor (s. vulgare), Theobroma cacao, Trifolium pratense, Triticum aestivum, Triticum durum, Vicia faba, Vitis vinifera and Zea mays.

In addition, the compounds I may also be used in crops which tolerate the action of herbicides owing to breeding, including genetic engineering methods.

Moreover, the 1-aryl-4-haloalkyl-2-[1H]-pyridones I and their agriculturally useful salts are also suitable for the desiccation and/or defoliation of plants.

As desiccants, they are suitable, in particular, for desiccating the above-ground parts of crop plants such as potatoes, oilseed rape, sunflowers and soybeans. This allows completely mechanical harvesting of these important crop plants.

Also of economic interest is the coordinated dehiscence of fruits or the reduction of their adherence to the plant, for example in citrus fruits, olives or other species of pomaceous fruit, stone fruit and nuts, since this facilitates harvesting of these fruits. Dehiscence is the result of the formation of abscission tissue between fruit or leaf and shoot of the plants, and is promoted by the compounds of the formula I according to the invention and their salts. Thus, the use of the compounds of the formula I according to the invention and their agriculturally useful salts permits coordinated dehiscence of fruits and also controlled defoliation of useful plants such as cotton, thus facilitating harvesting of such crop plants. Accordingly, controlled defoliation is of interest in particular in useful plants such as cotton. By shortening the interval in which the individual cotton plants mature, an improved quality of the harvested fiber material is achieved.

The compounds I, or the compositions comprising them, can be used for example in the form of ready-to-spray aqueous solutions, powders, suspensions, also highly-concentrated aqueous, oily or other suspensions or dispersions, emulsions, oil dispersions, pastes, dusts, materials for spreading, or granules, by means of spraying, atomizing, dusting, spreading, pouring, seed dressing or mixing with the seed. The use forms depend on the intended aims; in any case, they should ensure a very fine distribution of the active compounds according to the invention. The herbicidal compositions comprise a herbicidally effective amount of at least one compound of the formula I or an agriculturally useful salt of I and auxiliaries which are customary for formulating crop protection agents.

Suitable inert additives are essentially: Mineral oil fractions of medium to high boiling point, such as kerosene and diesel oil, furthermore coal tar oils and oils of vegetable or animal origin, aliphatic, cyclic and aromatic hydrocarbons, e.g. paraffins, tetrahydronaphthalene, alkylated naphthalenes and their derivatives, alkylated benzenes and their derivatives, alcohols such as methanol, ethanol, propanol, butanol and cyclohexanol, ketones such as cyclohexanone, strongly polar solvents, for example amines such as N-methylpyrrolidone, and water.

Aqueous use forms can be prepared from emulsion concentrates, suspensions, pastes, wettable powders or water-dispersible granules by adding water. To prepare emulsions, pastes or oil dispersions, the 1-aryl-4-haloalkyl-2-[1H]-pyridones either as such or dissolved in an oil or solvent, can be homogenized in water by means of a wetting agent, tackifier, dispersant or emulsifier. Alternatively, it is possible to prepare concentrates comprising active compound, wetting agent, tackifier, dispersant or emulsifier and, if desired, solvent or oil, which are suitable for dilution with water.

Suitable surfactants are the alkali metal salts, alkaline earth metal salts and ammonium salts of aromatic sulfonic acids, e.g. ligno-, phenol-, naphthalene- and dibutylnaphthalenesulfonic acid, and of fatty acids, alkyl- and alkylarylsulfonates, alkyl sulfates, lauryl ether sulfates and fatty alcohol sulfates, and salts of sulfated hexa-, hepta- and octadecanols, and also of fatty alcohol glycol ethers, condensates of sulfonated naphthalene and its derivatives with formaldehyde, condensates of naphthalene, or of the naphthalenesulfonic acids with phenol and formaldehyde, polyoxyethylene octylphenol ether, ethoxylated isooctyl-, octyl- or nonylphenol, alkylphenyl or tributylphenyl polyglycol ether, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignin-sulfite waste liquors or methylcellulose.

Powders, materials for spreading and dusts can be prepared by mixing or grinding the active substances together with a solid carrier.

Granules, for example coated granules, impregnated granules and homogeneous granules, can be prepared by binding the active compounds to solid carriers. Solid carriers are mineral earths, such as silicas, silica gels, silicates, talc, kaolin, limestone, lime, chalk, bole, loess, clay, dolomite, diatomaceous earth, calcium sulfate, magnesium sulfate, magnesium oxide, ground synthetic materials, fertilizers such as ammonium sulfate, ammonium phosphate and ammonium nitrate, ureas, and products of vegetable origin, such as cereal meal, tree bark meal, wood meal and nutshell meal, cellulose powders, or other solid carriers.

The concentrations of the active compounds I in the ready-to-use preparations can be varied within wide ranges. In general, the formulations comprise approximately from 0.001 to 98% by weight, preferably 0.01 to 95% by weight of at least one active compound. The active compounds are employed in a purity of from 90% to 100%, preferably 95% to 100% (according to the NMR spectrum).

The compounds I according to the invention can be formulated, for example, as follows:

I parts by weight of the compound No. IAe.131 are dissolved in a mixture composed of 80 parts by weight of alkylated benzene, 10 parts by weight of the adduct of 8 to 10 mol of ethylene oxide to 1 mol of oleic acid N-monoethanolamide, 5 parts by weight of calcium dodecylbenzenesulfonate and 5 parts by weight of the adduct of 40 mol of ethylene oxide to 1 mol of castor oil. Pouring the solution into 100,000 parts by weight of water and finely distributing it therein gives an aqueous dispersion which comprises 0.02% by weight of the active compound.

II 20 parts by weight of the compound No. IAa.128 are dissolved in a mixture composed of 40 parts by weight of cyclohexanone, 30 parts by weight of isobutanol, 20 parts by weight of the adduct of 7 mol of ethylene oxide to 1 mol of isooctylphenol and 10 parts by weight of the adduct of 40 mol of ethylene oxide to 1 mol of castor oil. Pouring the solution into 100,000 parts by weight of water and finely distributing it therein gives an aqueous dispersion which comprises 0.02% by weight of the active compound.

III 20 parts by weight of the active compound No. IAa.10 are dissolved in a mixture composed of 25 parts by weight of cyclohexanone, 65 parts by weight of a mineral oil fraction of boiling point 210 to 280° C. and 10 parts by weight of the adduct of 40 mol of ethylene oxide to 1 mol of castor oil. Pouring the solution into 100,000 parts by weight of water and finely distributing it therein gives an aqueous dispersion which comprises 0.02% by weight of the active compound.

IV 20 parts by weight of the active compound No. IAa.95 are mixed thoroughly with 3 parts by weight of sodium diisobutylnaphthalenesulfonate, 17 parts by weight of the sodium salt of a lignosulfonic acid from a sulfite waste liquor and 60 parts by weight of pulverulent silica gel, and the mixture is ground in a hammer mill. Finely distributing the mixture in 20,000 parts by weight of water gives a spray mixture which comprises 0.1% by weight of the active compound.

V 3 parts by weight of the active compound No. IAa.59 are mixed with 97 parts by weight of finely divided kaolin. This gives a dust which comprises 3% by weight of the active compound.

VI 20 parts by weight of the active compound No. IAa.22 (racemate) are mixed intimately with 2 parts by weight of calcium dodecylbenzenesulfonate, 8 parts by weight of fatty alcohol polyglycol ether, 2 parts by weight of the sodium salt of a phenol/urea/formaldehyde condensate and 68 parts by weight of a paraffinic mineral oil. This gives a stable oily dispersion.

VII 1 part by weight of the compound No. IAa.110 is dissolved in a mixture composed of 70 parts by weight of cyclohexanone, 20 parts by weight of ethoxylated isooctylphenol and 10 parts by weight of ethoxylated castor oil. This gives a stable emulsion concentrate.

VIII 1 part by weight of the compound No. IAa.131 is dissolved in a mixture composed of 80 parts by weight of cyclohexanone and 20 parts by weight of Wettol® EM 31 (=nonionic emulsifier based on ethoxylated castor oil). This gives a stable emulsion concentrate.

The herbicidal compositions or the active compounds can be applied pre- or post-emergence or together with the seed of a crop plant. It is also possible to apply the herbicidal compositions or active compounds by applying crop plant seed pretreated with the herbicidal compositions or active compounds. If the active compounds are less well tolerated by certain crop plants, application techniques may be used in which the herbicidal compositions are sprayed, with the aid of the spraying equipment, in such a way that they come into as little contact as possible, if any, with the leaves of the sensitive crop plants, while the active compounds reach the leaves of undesirable plants growing underneath, or the bare soil surface (post-directed, lay-by).

The rates of application of active compound are from 0.001 to 3.0, preferably 0.01 to 1.0, kg/ha of active substance (a.s.), depending on the control target, the season, the target plants and the growth stage.

To widen the spectrum of action and to achieve synergistic effects, the 1-aryl-4-haloalkyl-2-[1H]-pyridones may be mixed with a large number of representatives of other herbicidal or growth-regulating active compound groups and then applied concomitantly. Suitable components for mixtures are, for example, 1,2,4-thiadiazoles, 1,3,4-thiadiazoles, amides, aminophosphoric acid and its derivatives, aminotriazoles, anilides, (het)aryloxyalkanoic acid and its derivatives, benzoic acid and its derivatives, benzothiadiazinones, 2-aroyl-1,3-cyclohexanediones, 2-hetaroyl-1,3-cyclohexanediones, hetaryl aryl ketones, benzylisoxazolidinones, meta-CF₃-phenyl derivatives, carbamates, quinolinecarboxylic acid and its derivatives, chloroacetanilides, cyclohexenone oxime ether derivatives, diazines, dichloropropionic acid and its derivatives, dihydrobenzofurans, dihydrofuran-3-ones, dinitroanilines, dinitrophenols, diphenyl ethers, dipyridyls, halocarboxylic acids and their derivatives, ureas, 3-phenyluracils, imidazoles, imidazolinones, N-phenyl-3,4,5,6-tetrahydrophthalimides, oxadiazoles, oxiranes, phenols, aryloxy- and heteroaryloxyphenoxypropionic esters, phenylacetic acid and its derivatives, phenylpropionic acid and its derivatives, pyrazoles, phenylpyrazoles, pyridazines, pyridinecarboxylic acid and its derivatives, pyrimidyl ethers, sulfonamides, sulfonylureas, triazines, triazinones, triazolinones, triazolecarboxamides and uracils.

It may furthermore be advantageous to apply the compounds I, alone or else concomitantly in combination with other herbicides, in the form of a mixture with other crop protection agents, for example together with agents for controlling pests or phytopathogenic fungi or bacteria. Also of interest is the miscibility with mineral salt solutions, which are employed for treating nutritional and trace element deficiencies. Non-phytotoxic oils and oil concentrates may also be added.

The examples below serve to illustrate the invention:

### I PREPARATION EXAMPLES

### I.1 1-Aryl-2,6(1H,3H)-dihydropyridinediones of the Formula IIa

1. Preparation of Diethyl (2E)-3-Trifluoromethyl-2-pentenedicarboxylate (Intermediate a)

Over a period of one hour, 79.3 g (431 mmol) of ethyl trifluoroacetate were added to a solution of 150 g (431 mmol) of ethyl triphenylphosphoranylideneacetate in 500 ml of diethyl ether, and the mixture was kept at room temperature overnight. The resulting precipitate was filtered off and the filtrate was concentrated under reduced pressure. This gave 119 g of intermediate a which, according to ¹H-NMR, was still contaminated by triphenylphosphine oxide. The crude product was used without further purification for the subsequent steps.

¹H-NMR (CDCl₃, 270 MHz) δ [ppm]: 1.3 (2t, 6H), 3.75 (s, 2H), 4.2 (2q, 4H), 6.55 (s, 1H), 7.4-7.7 (triphenylphosphine oxide).

2. Preparation of (2E)-3-Trifluoromethyl-2-pentenedicarboxylic Acid

At room temperature, a solution of 37.9 g (948 mmol) of sodium hydroxide in 200 ml of water was added over a period of 20 minutes to a solution of 119 g (about 431 mmol) of intermediate a in 1 l of ethanol, and the mixture was stirred at room temperature overnight. The reaction mixture was concentrated under reduced pressure and the residue was then partitioned between 300 ml of water and 300 ml of ethyl acetate, the phases were separated and the aqueous phase was acidified to pH 1 using concentrated hydrochloric acid. The mixture was extracted three times with ethyl acetate, the combined organic phases were dried over magnesium sulfate and the organic phase was concentrated under reduced pressure. This gave 78.9 g of the dicarboxylic acid (intermediate b) as a colorless solid.

¹H-NMR (d₆-DMSO, 400 MHz) δ [ppm]: 3.6 (s, 2H), 6.55 (s, 1H).

3. Preparation of (2E)-3-Methyl- and (2E)-4-Methyl-3-trifluoromethyl-2-pentenedicarboxylic Acid

Using ethyl triphenylphosphoranylideneacetate and ethyl 2-(trifluoroacetyl)propionate as starting materials, the reaction according to the methods given for intermediates a and b gave (2E)-2-methyl-3-trifluoromethylpentenedicarboxylic acid and (2E)-4-methyl-3-trifluoromethyl-2-pentenedicarboxylic acid as a mixture of isomers in a molar ratio of 1:2. The mixture of isomers was used without further purification for preparing the compounds of the formula II (intermediate c).

4. Preparation of the Compounds of the Formula II

Isopropyl 2-Chloro-5-(2,6-dioxo-4-(trifluoromethyl)-3,6-dihydro-1(2H)-pyridinyl)-4-fluorobenzoate (Intermediate 1)

Method A

2.0 g (10 mmol) of intermediate b and 2.3 g (10 mmol) of isopropyl 5-amino-2-chloro-4-fluorobenzoate were heated at 160° C for 1.5 h. After cooling, this gave 3.7 g of intermediate 1 (see Table 3), corresponding to a yield of 94% of theory.

Method B

2.0 g (10 mmol) of intermediate b and 2.3 g (10 mmol) of isopropyl 5-amino-2-chloro-4-fluorobenzoate were dissolved in 40 ml of dichloromethane. The solvent was removed under reduced pressure. The resulting substance mixture was then heated at 700 W for 1 h and at 1000 W for 2 h in a commercial microwave. This gave the title compound in quantitative yield.

The intermediates 2 to 20 listed in Table 3 were prepared in a similar manner, using intermediate c instead of intermediate b for preparing the compounds 14 to 20. In each case, only 1 isomer was obtained.

The preparation of intermediate 4 was carried out by a modified method B where intermediate b and the O-ethyl oxime of 5-amino-2-chloro-4-fluorobenzaldehyde were reacted in xylene at 1000 W for 90 minutes.

TABLE 3
Compounds of the formula II where R³ = CF₃ and R⁴ = F; intermediates 1 to 20.

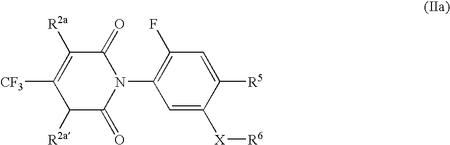

| Inter- | | | | | ¹H-NMR δ [ppm], CDCl₃, |
|---|---|---|---|---|---|
| mediate | R⁵ | R^{2a} | R^{2a′} | X—R⁶ | 270 MHz or 400 MHz |
| 1 | Cl | H | H | COO—CH(CH₃)₂ | δ 1.4 (6H), 3.8 (2H), 5.25 (1H), |
| | | | | | 6.8 (1H), 7.4 (1H), 7.8 (1H) |
| 2 | Cl | H | H | CH═C(Cl)—CO₂C₂H₅ | δ 1.4 (3H), 3.8 (2H), 4.4 (2H), |
| | | | | | 6.8 (sH), 7.4 (1H), 7.9 (1H), 8.1 (1H) |
| 3 | Cl | H | H | O—CH₂—C≡CH | δ 2.6 (1H), 3.75 (2H), 4.7 (2H), |
| | | | | | 6.8 (1H), 6.9 (1H), 7.3 (1H) |
| 4 | Cl | H | H | CH═N—OC₂H₅ | δ 1.3 (3H), 3.75 (2H), 4.2 (2H), |
| | | | | | 6.8 (sH), 7.3 (1H), 7.8 (1H), 8.4 (1H) |
| 5 | CN | H | H | O—CH₂—C≡CH | δ 2.6 (1H), 3.75 (2H), 4.8 (2H), |
| | | | | | 6.8 (1H), 7.0 (1H), 7.5 (1H) |
| 6 | Cl | H | H | COO-cyclo-C₅H₉ | |
| 7 | Cl | H | H | COO—CH(CH₃)—CO₂CH₃ | |
| | | | | | S enantiomer |
| 8 | Cl | H | H | COO—CH₂—C≡CH | |
| 9 | Cl | H | H | COO—CH₂—CH═CH₂ | |
| 10 | Cl | H | H | O-cyclo-C₅H₉ | |
| 11 | Cl | H | H | O—CH₃ | |
| 12 | CN | H | H | O—CH₃ | |
| 13 | Cl | H | H | O—CH(CH₃)—CO₂CH₃ | |
| | | | | racemate | |
| 14 | Cl | CH₃ | H | COO—CH(CH₃)₂ | |
| 15 | Cl | CH₃ | H | O—CH₂—C≡CH | |
| 16 | Cl | CH₃ | H | CH═N—OC₂H₅ | |
| 17 | Cl | CH₃ | H | O—CH(CH₃)—CO₂CH₃ | |
| | | | | racemate | |
| 18 | Cl | CH₃ | H | CH═C(Cl)—CO₂C₂H₅ | |
| 19 | Cl | CH₃ | H | COO—CH₂—CH═CH₂ | |
| 20 | Cl | CH₃ | H | COO—CH(CH₃)—CO₂CH₃ | |
| | | | | S enantiomer | |

### I.2 1-Aryl-2-(1H)-4-trifluoromethyl-6-chloropyridones (Examples 1 to 21)

Isopropyl 2-Chloro-5-[2-chloro-6-oxo-4-(trifluoromethyl)-1-(6H)-pyridinyl]-4-fluorobenzoate (Example 1)

2.3 g (5.8 mmol) of isopropyl 2-chloro-5-[2,6-dioxo-4-trifluoromethyl-3,6-dihydro-1-(2H)-pyrindinyl]-4-fluoro-benzoate (intermediate 1) were heated in 10 ml of phosphorus oxytrichloride (POCl₃) at reflux for 6 h. The mixture was allowed to cool overnight, excess phosphorus oxytrichloride was removed under reduced pressure and the crude product was purified by silica gel chromatography (cyclohexane/ethyl acetate). This gave 1.1 g of the title compound in a yield of 46%.

In a similar manner, the compounds of Examples 2-21 were prepared from intermediates 2-20 (see Table 4).

### I.3 1-Aryl-2-(1H)-4-trifluoromethylpyridones (Examples 22 to 26)

### Example 22

2,5-Difluoro-4-[2-oxo-4-(trifluoromethyl)-1-(2H)-pyridinyl)]benzonitrile

7.6 g (55.5 mmol) of potassium carbonate were added to a solution of 8.1 g (50 mmol) of 4-(trifluoromethyl)-2-pyridone in 100 ml of dimethylformamide. At room temperature, a solution of 8.6 g (55 mmol) of 2,4,5-trifluorobenzonitrile in 10 ml of dimethylformamide was then added. The mixture was heated at 80° C. for a total of 13 h. After cooling, the reaction mixture was concentrated under reduced pressure, the residue was dissolved in 400 ml of methyl tert-butyl ether and the organic phase was washed twice with water, dried over magnesium sulfate and treated under reduced pressure. The resulting crude product was purified by silica gel chromatography using a cyclohexane/ethyl acetate gradient (4:1 to 1:2). This gave 9.6 g of the title compound of melting point 150° C. The ¹H-NMR data of the compound are listed in Table 4.

### Example 23

5-Fluoro-2-methoxy-4-[2-oxo-4-(trifluoromethyl)-1-(2H)-pyridinyl]benzonitrile

0.6 g (2 mmol) of the compound from example 22 were dissolved in 60 ml of methanol, and 0.36 g (2.0 mmol) of a 30% by weight strength solution of sodium methoxide was added. The mixture was stirred at room temperature overnight and then concentrated to dryness under reduced pressure. The residue was purified by silica gel chromatography (MPLC) using the mobile phase cyclohexane/ethyl acetate (4:1). This gave 0.4 g (64% of theory) of the title compound of melting point 194-196° C. The ¹H-NMR spectrum of the compound is shown in Table 4.

### Example 24

5-Fluoro-4-[2-oxo-4-(trifluoromethyl)-1-(2H)-pyrindinyl)-2-(2-propinyloxy)]benzonitrile

0.16 g (4.0 mmol) of sodium hydride (60% in mineral oil) was added to a solution of 0.2 g (3.5 mmol) of propargyl alcohol in 50 ml of tetrahydrofuran. The mixture was stirred at room temperature for 10 minutes, and a solution of 1.0 g (3.3 mmol) of the compound from Example 22 in 20 ml of tetrahydrofuran was then added over a period of 10 minutes. The mixture was kept at room temperature overnight and then heated at reflux for 30 minutes. After cooling, the reaction mixture was concentrated under reduced pressure and the residue was purified by silica gel chromatography using a cylcohexane/ethyl acetate gradient. This gave 0.9 g of the slightly contaminated title compound. The impurities were removed by MPLC.

The compounds of Examples 25 and 26 were prepared in a similar manner.

### Example 27

4-Chloro-6-fluoro-7-[2-chloro-6-oxo-4-trifluoromethyl-1-(6H)-pyridinyl]-2-cyclopropyl-1,3-benzoxazole (Compound ICa.15)

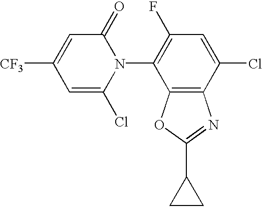

### 27.1

7-Chloro-6-fluoro-7-[2,6-dioxo-4-trifluoromethyl-3,6-dihydro-1-(2H)-pyridinyl]-2-cyclopropyl-1,3-benzoxazole

7-Chloro-6-fluoro-7-[2,6-dioxo-4-trifluoromethyl-3,6-dihydro-1-(2H)-pyridinyl]-2-cyclopropyl-1,3-benzoxazole was prepared from 7-amino-4-chloro-6-fluoro-2-cyclopropyl-1,3-benzoxazole and (2E)-3-trifluoromethyl-2-pentene dicarboxylic acid according to method A as an intermediate which was used without further purification in the following reaction.

### 27.2

4-Chloro-6-fluoro-7-[2-chloro-6-oxo-7-trifluoromethyl-1-(6H)-pyridinyl]-2-xyxlopropyl-1,3-benzoxazole

The title compound was obtained by means of the method described in example 1 from the compound of example 27.1 and phosphoroxitrichloride.

¹H-NMR (CDCl₃) δ: 1,2-1,4 (m, 4H, cPr), 2,2 (m, 1H, cPr), 6,6 (s, 1H, Pyridone-H), 6,95 (s, 1H, Pyridone-H), 7,3 (d, 1H, Ar—H).

TABLE 4
Compounds of the formula IAa where R³ = CF₃ and R⁴ = F; Examples 1 to 26.

| | |
|---|---|
| | (I) |
| 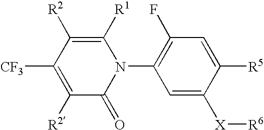 | |

| Ex. | No.¹⁾ | R¹ | R² | R^{2′} | R⁵ | X—R⁶ | ¹H-NMR(CDCl₃; 270 or 400MHz) |
|---|---|---|---|---|---|---|---|
| 1 | IAa.59 | Cl | H | H | Cl | COO—CH(CH₃)₂ | δ 1.45(d, 6H, CH(CH₃)₂], 5.3[septett, 1H, CH(CH₃)₂], 6.6(s, 1H, pyridone-H), 6.9(s, 1H, pyridone-H), 7.45(d, 1H, Ar—H), 7.85(d, 1H, Ar—H) |
| 2²⁾ | IAa.95 | Cl | H | H | Cl | CH═C(Cl)—CO₂C₂H₅ | δ 1.4(t, 3H, CH₂CH₃), 4.4(q, 2H, CH₂CH₃), 6.6(s, 1H, pyridone-H), 6.9(s, 1H, pyridone-H), 7.45(d, 1H, Ar—H), 7.95(d, 1H, Ar—H), 8.1[s, 1H, CH═C(Cl)COOEt |
| 3 | IAa.10 | Cl | H | H | Cl | O—CH₂—C≡CH | δ 2.6(t, 1H, C≡CH), 4.8(d, 2H, OCH₂C≡C), 6.55(s, 1H, pyridone-H), 6.9(s, 1H, pyridone-H), 7.0(d, 1H, Ar—H), 7.4(d, 1H, Ar—H) |
| 4 | IAa.110 | Cl | H | H | Cl | CH═N—OC₂H₅ | δ 1.3(t, 3H, CH₂CH₃), 4.2(q, 2H, CH₂CH₃), 6.55(s, 1H, pyridone-H), 6.9(s, 1H, pyridone-H), 7.35(d, 1H, Ar—H), 7.85(d, 1H, Ar—H), 8.4(s, 1H, CH═NOEt) |
| 5 | IAa.131 | Cl | H | H | CN | O—CH₂—C≡CH | δ 2.6(t, 1H, C≡CH), 4.8, 4.9(2dd, together 2H, OCH₂C≡C), 6.6(s, 1H, pyridone-H), 6.9(s, 1H, pyridone-H), 7.1(d, 1H, Ar—H), 7.55(d, 1H, Ar—H) |
| 6 | IAa.62 | Cl | H | H | Cl | COO-cyclopentyl | δ 1.6-2.0(m, 8H, cyclopentyl), 5.4(m, 1H, OCH), 6.6(s, 1H, pyridone-H), 6.9(s, 1H, pyridone-H), 7.4(d, 1H, Ar—H), 7.8(d, 1H, Ar—H) |
| 7⁴⁾ | IAa.769 | Cl | H | H | Cl | COO—CH(CH₃)—CO₂CH₃S enantiomer, mix- ture of rotational isomers | δ 1.6(d, 3H, CHCH₃), 3.8(s, 3H, OMe), 5.35(q, 1H, OCHCH₃), 6.6(s, 1H, pyri- done-H), 6.9(s, 1H, pyridone-H), 7.45(d, 1H, Ar—H), 8.0(d, 1H, Ar—H |
| 8 | IAa.61 | Cl | H | H | Cl | COO—CH₂—C≡CH | δ 2.55(t, 1H, C≡CH), 4.95(d, 2H, COOCH₂), 6.6(s, 1H, pyridone-H), 6.9(s,1H, pyridone-H), 7.45(d, 1H, Ar—H), 7.95(d, 1H, Ar—H) |
| 9 | IAa.60 | Cl | H | H | Cl | COO—CH₂—CH═CH₂ | δ 4.8(d, 2H, COOCH₂), 5.35, 5.45(2d, together 2H, allyl-H), 6.0(m, 1H, allyl-H), 6.6(s, 1H, pyridone-H), 6.9(s, 1H, pyri- done-H), 7.45(d, 1H, Ar—H), 7.95(d, 1H, Ar—H) |
| 10 | IAa.12 | Cl | H | H | Cl | O-cyclopentyl | δ δ = 1.6-2.0(m, 8H, cyclopentyl), 4.75(m, 1H, OCH), 6.55(s, 1H, pyridone-H), 6.75(d, 1H, Ar—H), 6.9(s, 1H, pyridone- H), 7.35(d, 1H, Ar—H) |
| 11 | IAa.7 | Cl | H | H | Cl | O—CH₃ | δ 3.9(s, 3H, OMe), 6.55(s, 1H, pyridone- H), 6.8(d, 1H, Ar—H), 6.9(s, 1H, pyri- done-H), 7.35(d, 1H, Ar—H), m.p. 162-163° C. |
| 12 | IAa.128 | Cl | H | H | CN | O—CH₃ | δ 3.95(s, 3H, OMe), 6.55(s, 1H, pyri- done-H), 6.9(d, 1H, Ar—H), 6.9(s, 1H, py- ridone-H), 7.55(d, 1H, Ar—H) m.p. 191-196° C. |
| 13⁴⁾ | IAa.22 | Cl | H | H | Cl | O—CH(CH₃)—CO₂CH₃racemate, mixture of rotational isomers | δ 1.7(d, 3H, CHCH₃), 3.75(s, 3H, COOMe), 4.7(q, 1H, CHCH₃), 6.55(s, 1H, pyridone-H), 6.75(isomer A) or 6.8(iso- mer B) (d, 1H, Ar—H), 6.9(s, 1H, pyri- done-H), 7.4(d, 1H, Ar—H) |
| 14 | IAb.59 | Cl | H | CH₃ | Cl | COO—CH(CH₃)₂ | δ 1.4(d, 6H, CH(CH₃)₂], 2.3(s, 3H, CH₃, pyridone), 5.25[septett, 1H, CH(CH₃)₂], 6.6(s, 1H, pyridone-H), 7.4(d, 1H, Ar—H), 7.8(d, 1H, Ar—H) |
| 15 | IAb.10 | Cl | H | CH₃ | Cl | O—CH₂—C≡CH | δ 2.3(s, 3H, CH₃, pyridone), 2.6(t, 1H, C≡CH), 4.8(d, 2H, OCH₂C≡C), 6.6(s, 1H, pyridone-H), 7.0(d, 1H, Ar—H), 7.4(d, 1H, Ar—H); melting point 91-92° C. |
| 16 | IAb.110 | Cl | H | CH₃ | Cl | CH═N—OC₂H₅ | δ 1.3(t, 3H, CH₂CH₃), 2.3(s, 3H, CH₃, pyridone), 4.2(q, 2H, CH₂CH₃), 6.6(s, 1H, pyridone-H), 7.35(d, 1H, Ar—H), 7.85(d, 1H, Ar—H), 8.4(s, 1H, CH═NOEt) |
| 17⁴⁾ | IAb.22 | Cl | H | CH₃ | Cl | O—CH(CH₃)—CO₂CH₃racemate, mixture of rotational isomers | δ 1.7(d, 3H, CHCH₃), 2.3(s, 3H, CH₃, pyridone), 3.75(s, 3H, COOMe), 4.7(q, 1H, CHCH₃), 6.6(s, 1H, pyridone-H), 6.75(isomer A) or 6.85(isomer B) (d, 1H, Ar—H), 7.35(d, 1H, Ar—H) |
| 18²⁾ | IAb.95 | Cl | H | CH₃ | Cl | CH═C(Cl)—CO₂C₂H₅ | δ 1.4(t, 3H, CH₂CH₃), 2.3(s, 3H, CH₃, pyridone), 4.4(q, 2H, CH₂CH₃), 6.6(s, 1H, pyridone-H), 7.4(d, 1H, Ar—H), 7.95(d, 1H, Ar—H), 8.1[s, 1H, CH═C(Cl)COOEt] |
| 19^{2) 3)} | IAc.95 | Cl | CH₃ | H | Cl | CH═C(Cl)—CO₂C₂H₅ | δ 1.4(t, 3H, CH₂CH₃), 2.3(s, 3H, CH₃, pyridone), 4.4(q, 2H, CH₂CH₃), 7.0(s, 1H, pyridone-H), 7.4(d, 1H, Ar—H), 7.9(d, 1H, Ar—H), 8.1[s, 1H, CH═C(Cl)COOEt] |
| 20 | IAb.60 | Cl | H | CH₃ | Cl | COO—CH₂—CH═CH₂ | δ 2.3(s, 3H, CH₃, pyridone), 4.8(d, 2H, COOCH₂), 5.35, 5.45(2d, together 2H, allyl-H), 6.0(m, 1H, allyl-H), 6.6(s, 1H, pyridone-H), 7.4(d, 1H, Ar—H), 7.9(d, 1H, Ar—H) |
| 21⁴⁾ | IAb.769 | Cl | H | CH₃ | Cl | COO—CH(CH₃)—CO₂CH₃S enantiomer, mix- ture of rotational isomers | δ 1.6(d, 3H, CHCH₃), 2.3(s, 3H, CH₃, pyridone), 3.8(s, 3H, OMe), 5.35(q, 1H, OCHCH₃), 6.6(s, 1H, pyridone-H), 7.45(d, 1H, Ar—H), 8.0(d, 1H, Ar—H) |
| 22 | IAe.123 | H | H | H | CN | F | δ 6.45(d, 1H), 7.0(s, 1H), 7.3-7.4(m, 2H), 7.6(dd, 1H); melting point 150° C. |
| 23 | IAe.128 | H | H | H | CN | OCH₃ | δ 3.95(s, 3H, OMe), 6.45(d, 1H), 7.0(s, 1H), 7.05(d, 1H), 7.4(d, 1H), 7.5(d, 1H); melting point 194-196° C. |
| 24 | IAe.131 | H | H | H | CN | O—CH₂—C≡CH | δ 2.6(t, 1H, C≡CH), 4.85(d, 2H, OCH₂C≡C), 6.45(dd, 1H), 7.0(s, 1H), 7.2(d, 1H), 7.4(d, 1H), 7.55(d, 1H) |
| 25 | IAe.132 | H | H | H | CN | O—CH(CH₃)—C≡CH | d 1.8(d, 3H, OCH(CH₃)—C≡CH), 2.6(d, 1H, C≡CH), 4.9(dq, 1H, OCH(Me)C≡CH, 6.45(dd, 1H), 7.0(s, 1H), 7.2(d, 1H), 7.35(d, 1H), 7.5(d, 1H); melting point 176-181° C. |
| 26 | IAe.143 | H | H | H | CN | O—CH(CH₃)—CO₂CH₃racemate | δ 1.75(d, 3H, OCH(CH₃)COOMe), 3.8(s, 3H, COOCH₃), 4.8(q, 1H, OCH(CH₃)COOMe), 6.45(dd, 1H), 6.9(d, 1H), 6.95(s, 1H), 7.3(d, 1H), 7.55(d, 1H); melting point 131-133° C. |

¹⁾Numeration according to Table 1
²⁾Z enantiomer (phenyl ring with respect to halogen atom)
³⁾Byproduct in the preparation of Ib.95 (Example 18)
⁴⁾Mixture of diastereomers (Examples 7, 13, 17, 21)

### II USE EXAMPLES

### The Herbicidal Action of the 1-Aryl-4-haloalkyl-2-[1H]pyridones of the Formula I was Demonstrated by Greenhouse Experiments

The culture containers used were plastic pots with loamy sand containing approximately 3.0% of humus as the substrate. The seeds of the test plants were sown separately for each species.

For the pre-emergence treatment, the active compounds, which had been suspended or emulsified in water, were applied directly after seeding by means of finely distributing nozzles. The containers were irrigated gently to promote germination and growth and subsequently covered with transparent plastic hoods until the plants had taken root. This cover causes uniform germination of the test plants unless this was not adversely affected by the active compounds.

For the post-emergence treatment, the test plants were initially grown to a height of 3 to 15 cm, depending on the habit, and then treated with the active compounds which had been suspended or emulsified in water. To this end, the test plants were either sown directly and cultivated in the same containers, or they were initially cultivated separately as seedlings and transplanted into the test containers a few days prior to the treatment. The application rate for the post-emergence treatment was 0.0313 and 0.0156 kg of a. S./ha.

The plants were kept at temperatures of 10-25° C. and 20-35° C., depending on the species. The test period extended over 2 to 4 weeks. During this time, the plants were tended, and their reaction to the individual treatments was evaluated.

Evaluation was carried out using a scale from 0 to 100. 100 means no emergence of the plants, or complete destruction of at least the above-ground parts, and 0 means no damage or normal course of growth.

The plants used in the greenhouse experiments were of the following species:

| Bayer code | Common name |
|---|---|
| ABUTH | velvet leaf |
| AMARE | redroot pigweed |
| COMBE | dayflower |
| GALAP | catchweed bedstraw |
| SETFA | giant foxtail |

Here, the compound from Example 1 (No. IAa.59) showed very good activity against the harmful plants mentioned.

Use Examples (Desiccant/Defoliant Action)

The test plants used were young cotton plants with 4 leaves (without cotyledons) which had been grown under greenhouse conditions (relative atmospheric humidity 50-70%; day/night temperature 27/20° C.).

The young cotton plants were subjected to folia treatment to run-off point with aqueous preparations of the active compounds (with addition of 0.15% by weight, based on the spray mixture, of the fatty alcohol alkoxylate Plurafac® LF 700). The amount of water applied was 1000 l/ha (converted). After 13 days, the number of leaves shed and the degree of defoliation in % were determined.

The untreated control plants did not shed any leaves.

We claim:

1. The use of 1-aryl-4-haloalkyl-2-[1H]-pyridones of the formula I 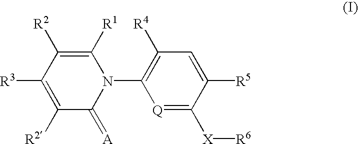
in which variables A, X, Q, R¹, R², R^{2′}, R³, R⁴, R⁵ and R⁶ are as defined below:
R¹ is chlorine;
R² and R^{2′} independently of one another are hydrogen, amino or C₁-C₄-alkyl;
R³ is trifluoromethyl;
R⁴ is hydrogen or halogen;
R⁵ is hydrogen, cyano, halogen, C₁-C₄-alkyl, C₁-C₄-haloalkyl, C₁-C₄-alkoxy or C₁-C₄-haloalkoxy;
A is oxygen or sulfur;
X is a chemical bond, methylene, 1,2-ethylene, propane-1,3-diyl, ethene-1,2-diyl, ethyne-1,2-diyl or is oxymethylene or thiamethylene, attached to the phenyl ring via the heteroatom, where all groups may be unsubstituted or may carry one or two substituents, in each case selected from the group consisting of cyano, carboxyl, halogen, C₁-C₄-alkyl, C₁-C₄-haloalkyl, C₁-C₄-alkoxy, (C₁-C₄-alkoxy)carbonyl, di(C₁-C₄-alkyl)amino and phenyl;
R⁶ is hydrogen, nitro, cyano, halogen, halosulfonyl, —O—Y—R⁸, —O—CO—Y—R⁸, —N(Y—R⁸)(Z—R⁹), —N(Y—R⁸)—SO₂—Z—R⁹, —N(SO₂—Y—R⁸)(SO₂—Z—R⁹), —N(Y—R⁸)—CO—Z—R⁹, —N(Y—R⁸)(O—Z—R⁹), —S(O)ₙ—Y—R⁸ where n=0, 1 or 2, —SO₂—O—Y—R⁸, —SO₂—N(Y—R⁸)(Z—R⁹), —CO—Y—R⁸, —C(═NOR¹⁰)—Y—R⁸, —C(═NOR¹⁰)—O—Y—R⁸, —CO—O—Y—R⁸, —CO—S—Y—R⁸, —CO—N(Y—R⁸)(Z—R⁹), —CO—N(Y—R⁸)(O—Z—R⁹) or —PO(O—Y—R⁸)₂;
Q is nitrogen or a group C—R⁷ in which R⁷ is hydrogen, OH, SH or NH₂; or p1 X—R⁶ and R⁷ are a 3- or 4-membered chain whose chain members may, in addition to carbon, include 1, 2 or 3 heteroatoms selected from the group of nitrogen, oxygen and sulfur atoms, which may be unsubstituted or may for their part carry one, two or three substituents and whose members may also include one or two nonadjacent carbonyl, thiocarbonyl or sulfonyl groups,
where the variables Y, Z, R⁸, R⁹ and R¹⁰ are as defined below:
Y, Z independently of one another are:
a chemical bond, methylene or 1,2-ethylene, which may be unsubstituted or may carry one or two substituents, in each case selected from the group consisting of carboxyl, C₁-C₄-alkyl, C₁-C₄-haloalkyl, (C₁-C₄-alkoxy)carbonyl and phenyl;
R⁸, R⁹ independently of one another are:
hydrogen, C₁-C₆-haloalkyl, C₁-C₄-alkoxy-C₁-C₄-alkyl, C₂-C₆-alkenyl, C₂-C₆-haloalkenyl, C₂-C₆-alkynyl, C₂-C₆-haloalkynyl, —CH(R¹¹)(R¹²), —C(R¹¹)(R¹²)—NO₂, —C(R¹¹)(R¹²)—CN, —C(R¹¹)(R¹²)halogen, —C(R¹¹)(R¹²)—OR¹³, —C(R¹¹)(R¹²)—N(R¹³)R¹⁴, —C(R¹¹)(R¹²)—N(R¹³)—OR¹⁴, —C(R¹¹)(R¹²)—SR¹³, —C(R¹¹)(R¹²)—SO—R¹³, —C(R¹¹)(R¹²)—SO₂—R¹³, —C(R¹¹)(R¹²)—SO₂—OR¹³, —C(R¹¹)(R¹²)—SO₂—N(R¹³)R¹⁴, —C(R¹¹)(R¹²)—CO—R¹³, —C(R¹¹)(R¹²)—C(═NOR¹⁵)—R¹³, —C(R¹¹)(R¹²)—CO—OR¹³, —C(R¹¹)(R¹²)—CO—SR¹³, —C(R¹¹)(R¹²)—CO—N(R¹³)R¹⁴, —C(R¹¹)(R¹²)—CO—N(R¹³)—OR¹⁴, —C(R¹¹)(R¹²)—PO(OR¹³)₂, C₃-C₈-cycloalkyl-C₁-C₄-alkyl, C₃-C₈-cycloalkyl which may contain a carbonyl or thiocarbonyl ring member,
phenyl or 3-, 4-, 5-, 6- or 7-membered heterocyclyl which may contain a carbonyl or thiocarbonyl ring member, where each cycloalkyl, the phenyl and each heterocyclyl ring may be unsubstituted or may carry one, two, three or four substituents, in each case selected from the group consisting of cyano, nitro, amino, hydroxyl, carboxyl, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfonyl, C₁-C₄-haloalkylsulfonyl, (C₁-C₄-alkyl)carbonyl, (C₁-C₄-haloalkyl)carbonyl, (C₁-C₄-alkyl)carbonyloxy, (C₁-C₄-haloalkyl)carbonyloxy, (C₁-C₄-alkoxy)carbonyl and di(C₁-C₄-alkyl)amino;
R¹⁰ is hydrogen, C₁-C₆-alkyl, C₁-C₆-haloalkyl, C₁-C₄-alkoxycarbonyl-C₁-C₄-alkyl, C₂-C₆-alkenyl, C₂-C₆-haloalkenyl, C₂-C₆-alkynyl, C₂-C₆-haloalkynyl, C₃-C₈-cycloalkyl, phenyl or phenyl-C₁-C₄-alkyl;
where the variables R¹¹ to R¹⁵ are as defined below:
R¹¹, R¹² independently of one another are
hydrogen, C₁-C₄-alkyl, C₁-C₄-alkoxy-C₁-C₄-alkyl, C₁-C₄-alkylthio-C₁-C₄-alkyl, (C₁-C₄-alkoxy)carbonyl-C₁-C₄-alkyl or phenyl-C₁-C₄-alkyl, where the phenyl ring may be unsubstituted or may carry one to three substituents, in each case selected from the group consisting of cyano, nitro, carboxyl, halogen, C₁-C₄-alkyl, C₁-C₄-haloalkyl and (C₁-C₄-alkoxy)carbonyl;
R¹³, R¹⁴ independently of one another are
hydrogen, C₁-C₆-alkyl, C₁-C₆-haloalkyl, C₁-C₄-alkoxy-C₁-C₄-alkyl, C₂-C₆-alkenyl, C₂-C₆-haloalkenyl, C₂-C₆-alkynyl, C₂-C₆-haloalkynyl, C₃-C₈-cycloalkyl, C₃-C₈-cycloalkyl-C₁-C₄-alkyl, phenyl, phenyl-C₁-C₄-alkyl, 3- to 7-membered heterocyclyl or heterocyclyl-C₁-C₄-alkyl, where each cycloalkyl and each heterocyclyl ring may contain a carbonyl or thiocarbonyl ring member,
and where each cycloalkyl, the phenyl and each heterocyclyl ring may be unsubstituted or may carry one to four substituents, in each case selected from the group consisting of cyano, nitro, amino, hydroxyl, carboxyl, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfonyl, C₁-C₄-haloalkylsulfonyl, (C₁-C₄-alkyl)carbonyl, (C₁-C₄-haloalkyl)carbonyl, (C₁-C₄-alkyl)carbonyloxy, (C₁-C₄-haloalkyl)carbonyloxy, (C₁-C₄-alkoxy)carbonyl and di(C₁-C₄-alkyl)amino;
R¹⁵ is hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, phenyl or phenyl-C₁-C₄-alkyl;
and their agriculturally useful salts as herbicides or for the desiccation/defoliation of plants.

2. The use as claimed in claim 1, with R⁵ in formula I having the following meaning:
R⁵ is halogen or cyano.

3. The use as claimed in claim 2, with Q in formula I being N or CH.

4. A 1-aryl-4-haloalkyl-2-[1H]-pyridone of formula I as defined in claim 1, wherein the variables A, X, Q, R², R^{2′} and R⁵ are as defined in claim 1 and the variables R¹, R³, R⁴. R⁵ and R⁶ are as defined below:
R¹ is chlorine;
R³ is trifluoromethyl;
R⁴ is halogen;
R⁵ is halogen or cyano;
R⁶ is hydrogen, nitro, cyano, halogen, halosulfonyl, —O—Y—R⁸, —O—CO—Y—R⁸, —N(Y—R⁸)(Z—R⁹), —N(Y—R⁸)—SO₂—Z—R⁹, —N(SO₂—Y—R⁸)(SO₂—Z—R⁹), —N(Y—R⁸)—CO—Z—R⁹, —N(Y—R⁸)(O—Z—R⁹), —S(O)ₙ—Y—R⁸ where n=0, 1 or 2, —SO₂—O—Y—R⁸, —SO₂—N(Y—R₈)(Z—R⁹), —CO—Y—R⁸, —C(═NOR¹⁰)—Y—R⁸, —C(═NOR¹⁰) —O—Y—R⁸, —CO—O—Y—R⁸, —CO—S—Y—R⁸, —CO—N(Y—R⁸)(Z—R⁹), —CO—N(Y—R⁸)(O—Z—R⁹) or —PO(O—Y—R⁸)₂;
X—R⁶ and R⁷ are a 3- or 4-membered chain whose chain members `may, in addition to carbon, include 1, 2 or 3 heteroatoms selected from the group of nitrogen, oxygen and sulfur atoms, which may be unsubstituted or may for their part carry one, two or three substituents and whose members may also include one or two nonadjacent carbonyl, thiocarbonyl or sulfonyl groups;
and their agriculturally useful salts.

5. A 1-aryl-4-haloalkyl-2-[1H]-pyridone as claimed in claim 4 of the formula I in which Q is nitrogen or CH.

6. A 1-aryl-4-haloalkyl-2-[1H]-pyridone as claimed in claim 4 of the formula I in which Q is C—R⁷ and R⁷ together with —X—R⁶ is a chain of the formulae O—C(R¹⁶,R¹⁷)—CO—N(R¹⁸)—, S—C(R¹⁶,R¹⁷)—CO—N(R¹⁸)—, N═C(R¹⁹)—O— or N═C(R¹⁹)—S—, where the variables R¹⁶ to R¹⁹ are as defined below:
R¹⁶, R¹⁷ independently of one another are hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, phenyl or phenyl-C₁-C₄-alkyl;
R¹⁸ is hydrogen, hydroxyl, C₁-C₆-alkyl, C₁-C₆-haloalkyl, C₂-C₆-alkenyl, C₂-C₆-haloalkenyl, C₂-C₆-alkynyl, C₁-C₄-alkoxy, C₁-C₄-haloalkoxy, C₃-C₆-alkenyloxy, C₃-C₆-alkynyloxy, C₁-C₄-alkylsulfonyl, C₁-C₄-haloalkylsulfonyl, C₁-C₄-alkylcarbonyl, C₁-C₄-haloalkylcarbonyl, C₁-C₄-alkoxycarbonyl, C₁-C₄-alkoxy-C₁-C₄-alkyl, C₁-C₄-alkoxycarbonyl-C₁-C₄-alkyl, C₁-C₄-alkoxycarbonyl-C₁-C₄-alkoxy, di (C₁-C₄-alkyl) aminocarbonyl, di (C₁-C₄-alkyl) aminocarbonyl-C₁-C₄-alkyl, di(C₁-C₄-alkyl)aminocarbonyl-C₁-C₄-alkoxy, phenyl, phenyl-C₁-C₄-alkyl, C₃-C₈-cycloalkyl, C₃-C₈-cycloalkyl-C₁-C₄-alkyl, 3-, 4-, 5-, 6- or 7-membered heterocyclyl which contains one or two ring heteroatoms selected from the group consisting of oxygen, nitrogen and sulfur,
R¹⁹ is hydrogen, halogen, cyano, amino, C₁-C₆-alkyl, C₁-C₆-haloalkyl, C₂-C₆-alkenyl, C₂-C₆-haloalkenyl, C₂-C₆-alkynyl, C₁-C₄-alkoxy, C₁-C₄-haloalkoxy, C₃-C₆-alkenyloxy, C₃-C₆-alkynyloxy, C₁-C₄-alkylamino, di(C₁-C₄-alkyl)amino, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₁-C₄-alkylcarbonyl, C₁-C₄-haloalkylcarbonyl, C₁-C₄-alkoxy-C₁-C₄-alkyl, C₁-C₄-alkoxycarbonyl, C₁-C₄-alkoxycarbonyl-C₁-C₄-alkyl, C₁-C₄-alkoxycarbonyl-C₁-C₄-alkoxy, C₁-C₄-alkoxycarbonyl-C₁-C₄-alkylthio, di(C₁-C₄-alkyl)aminocarbonyl, di (C₁-C₄-alkyl) aminocarbonyl-C₁-C₄-alkyl, di(C₁-C₄-alkyl)aminocarbonyl-C₁-C₄-alkoxy, di(C₁-C₄-alkyl) aminocarbonyl-C₁-C₄-alkylthio, C₃-C₈-cycloalkyl, phenyl, phenyl-C₁-C₄-alkyl, C₃-C₈-cycloalkyl-C₁-C₄-alkyl, 3-, 4-, 5-, 6- or 7-membered heterocyclyl which contains one or two ring heteroatoms selected from the group consisting of oxygen, nitrogen and sulfur.

7. A 1-aryl-4-haloalkyl-2-[1H]-pyridone as claimed in any of claims 4 to 6 in which R² and R^{2′} independently of one another are hydrogen or methyl.

8. A composition, comprising a herbicidally effective amount of at least one 1-aryl-4-haloalkyl-2-[1H]-pyridone of the formula I defined in claim 4 or an agriculturally useful salt of I and at least one inert liquid and/or solid carrier and, if desired, at least one surfactant.

9. A composition for the desiccation and/or defoliation of plants, comprising such an amount of at least one 1-aryl-4-haloalkyl-2-[1H]-pyridone of the formula I defined in claim 4 or of an agriculturally useful salt of I, as set forth in claim 1, that it has desiccant and/or defoliant action, and at least one inert liquid and/or solid carrier and, if desired, at least one surfactant.

10. A method for controlling undesirable vegetation, which comprises allowing a herbicidally effective amount of at least one 1-aryl-4-haloalkyl-2-[1H]-pyridone of the formula I defined in claim 1 or of an agriculturally useful salt of I to act on plants, their habitat or seed.

11. A method for the desiccation and/or defoliation of plants, which comprises allowing such an amount of at least one 1-aryl-4-haloalkyl-2-[1H]-pyridone of the formula I defined in claim 1 or of an aqriculturally useful salt of I that it has desiccant and/or defoliant action to act on plants.

12. A method as claimed in claim 11, wherein cotton is treated.

13. A compound of the formula II for preparing a 1-aryl-4-haloalkyl-2-[1H]-pyridone, 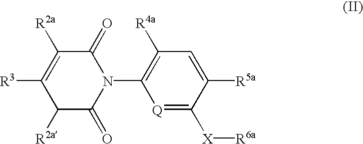
in which R³, X and Q are as defined in claim 4 and R^{2a}, R^{2a′}, R^{4a}, R^{5a}, R^{6a} are R², R^{2′}, R⁴, R⁵ and R⁶ as defined in claim 4.
